(19) United States
(12) Patent Application Publication
(10) Pub. No.: US 2025/0302957 A1
(43) Pub. Date: Oct. 2, 2025

(54) INSULIN TREATMENT TO IMPROVE T CELL ENGINEERING

(71) Applicant: GENENTECH, INC., South San Francisco, CA (US)

(72) Inventors: Shahram MISAGHI, South San Francisco, CA (US); David SHAW, South San Francisco, CA (US); Shuya WANG, South San Francisco, CA (US)

(21) Appl. No.: 19/233,993

(22) Filed: Jun. 10, 2025

Related U.S. Application Data
(63) Continuation of application No. PCT/US2023/083628, filed on Dec. 12, 2023.
(60) Provisional application No. 63/387,068, filed on Dec. 12, 2022.
(60) Provisional application No. 63/608,697, filed on Dec. 11, 2023.

(51) Int. Cl. A61K 40/11; A61K 40/32; A61K 40/42; C07K 14/725; C07K 16/32; C12N 5/0783

(52) U.S. Cl. CPC A61K 40/11; A61K 40/32; A61K 40/4238; C07K 14/7051; C07K 16/32; C12N 5/0636; C12N 2501/2307; C12N 2501/2315; C12N 2501/33

(57) ABSTRACT

Provided herein, inter alia, are methods and compositions for engineering T cells. The methods include culturing a T cell with insulin during engineering of the T cell. The methods provided herein are contemplated to increase cell viability, expansion and gene editing efficiency, thereby allowing an increase in the total number of engineered T cells.

### CROSS-REFERENCE TO RELATED APPLICATIONS

This application is an International Application which claims priority to and benefit of U.S. Provisional Application No. 63/387,068 filed Dec. 12, 2022 and U.S. Provisional Application No. 63/608,697 filed Dec. 11, 2023, the contents of each of which are incorporated herein by reference in their entireties and for all purposes.

### BACKGROUND

Genetic engineering has nearly limitless potential as a tool to improve human health. Indeed, genetic engineering has introduced and transformed therapies in every aspect of medical practice today. Of particular interest is the genetic engineering of T cells. T cell based therapies have become powerful new medicines, especially in the realms of cancer and immune system regulation. However, significant hurdles remain with respect to improving the viability and expansion capacity of genetically engineered T cells.

Disclosed herein, inter alia, are solutions to these and other problems in the art.

### BRIEF SUMMARY OF THE INVENTION

In an aspect, provided herein, is a method of editing an endogenous gene in a population of T cells, the method including: contacting the population of T cells with a gene editing reagent or a polynucleotide encoding a gene editing reagent under conditions to allow the polynucleotide or gene editing reagent to enter the cell, and culturing the population of T cells in the presence of one or more of, insulin, an insulin analog, an insulin agonist and/or an insulin partial agonist before and/or during, and/or after the contacting step to obtain an engineered population of T cells.

In another aspect, provided herein, is a method of monitoring of cell viability for an engineered population of T cells, the method comprising measuring mitochondrial function and cell metabolism over time.

In another aspect, provided herein, is a method of monitoring cell viability for an engineered population of T cells, the method comprising measuring a cell metabolism marker over time.

In another aspect, provided herein, is a method of increasing cell viability of a population of engineered T cells, including contacting a population of T cells in the presence of insulin, an insulin analog, an insulin agonist, an insulin partial agonist, a gene editing reagent, or a polynucleotide encoding a gene editing reagent thereby forming the population of engineered T cells, wherein the population of engineered T cells has increased cell viability, growth, and/or gene editing efficiencies relative to a population of engineered T cells that are not contacted with insulin, insulin analog, insulin agonist, or insulin partial agonist, wherein the population of engineered T cells are administered to a subject in need thereof.

In another aspect, provided herein, is a method of increasing gene editing efficiency in a population of engineered T cells, including contacting a population of T cells with insulin, an insulin analog, an insulin agonist, an insulin partial agonist, a gene editing reagent, and a polynucleotide, thereby forming the population of engineered T cells, wherein the population of engineered T cells has increased gene editing efficiency relative to a population of engineered T cells that are not contacted with insulin, insulin analog, insulin agonist, or insulin partial agonist.

A method for increasing expansion of a population of engineered T cells, including: (i) contacting a population of T cells with insulin, an insulin analog, an insulin agonist, and/or an insulin partial agonist and a polynucleotide, thereby forming the population of engineered T cells, and (ii) expanding the population of engineered T cells, thereby forming a population of expanded engineered T cells, wherein the insulin, insulin analog, insulin agonist, and/or insulin partial agonist increases the population of expanded engineered T cells relative to a population of engineered T cells that are not contacted with insulin, insulin analog, insulin agonist, and/or insulin partial agonist.

In another aspect, provided herein, is an engineered population of T cells, made by the method provided herein including embodiments thereof.

In another aspect, provided herein, is a pharmaceutical composition comprising the engineered T cell provided herein including embodiments thereof.

In another aspect, provided herein, is a method of treating a disease in a subject in need thereof, comprising administering a therapeutically effective amount of the engineered T cell provided herein including embodiments thereof or the pharmaceutical composition provided herein including embodiments thereof.

### BRIEF DESCRIPTION OF THE DRAWINGS

FIG. 1. Insulin treatment stimulates Akt phosphorylation in T cells. Western blot analysis of Akt, Erk and STAT5 and their respective phosphorylated forms in activated human CD8+ T cells with (+Insulin) or without (Control) insulin pre-treatment. Samples were obtained 15, 30, and 180 minutes (min) post insulin treatment in media without glucose. All measurements were performed using Image Lab software. C-Cas3: cleaved Caspase 3.

FIGS. 2A-2F. Insulin pre-treatment (24 h) of cultured T cells prior to electroporation improves culture viability, expansion, and total edited cells (TECs) levels. FIGS. 2A-2C show T cell final drug product (FDP) expansion folds after a 15-day T cell engineering process with different pre-transfection (TFX) insulin treatment concentrations, calculated based on seeding density on day 2. Data obtained from 3 individual donors pre-treated with different concentrations of insulin. FIGS. 2D-2F show total edited cells (TEC) number in FDP was calculated for the T cells engineered as indicated in FIGS. 2A-2C. Numbers in each column are rounded up to whole integers. TEC was calculated by multiplying the expansion fold with the knock-in percentage and was based on 1 million cell seeding density post-transfection per group. Insulin concentrations used: Low: 1 μg/ml, Mid: 5 μg/ml, High: 25 μg/ml. CTR: control.

FIGS. 3A-3E. Insulin pre-treatment enhances expression/activation of Akt and Erk signaling pathways in T cells. Western blot of Akt and Erk expression and phosphorylation with (Insulin Pre-24 h) or without (CTR) insulin pre-treatment (FIG. 3A). FIGS. 3B-3E show quantification of Western blot data for Akt and Erk expression and phosphorylation. Total Akt (FIG. 3B) and Erk (FIG. 3D) expression was calculated from the ratio of the protein band intensity to the internal control (actin) band intensity. Protein phosphorylation levels were calculated from the ratio of Akt (FIG. 3C) or Erk (FIG. 3E) phosphorylation band intensity to the total Akt of Erk protein band intensity. ON, 2N represent day 0 and day 2 (prior to transfection), respectively; 2, 3, 4, 6, 8, 11, 15 (or 2 T, 3 T, 4 T, 6 T, 8 T, 11 T, 15 T) represent days post ON (TFX on day 2 of the culture). Western blots were imaged using Image Lab software and band intensities were analyzed by ImageJ.

FIGS. 4A-4E. Insulin Treatment enhances survival of edited T cells and thus levels of T cell editing in the FDP both pre-TFX and post-TFX. FIGS. 4A and 4B show knock-in percentages when the cultures were treated with the indicated insulin concentrations pre-TFX from 2 independent donors compared with untreated control (CTR). FIGS. 4C and 4D show knock-in percentages when the cultures were treated with the indicated insulin concentrations post-TFX from 2 independent donors compared with untreated control (CTR). Indicated insulin doses pre-TFX were used 24 h prior to transfection (Pre-Treat) or 6 days post TFX (Post-Treat). Samples were collected on day 15 and analyzed by flow cytometry. Knock-in ratio was measured by using MHC-peptide dextramer staining (Dex) during flow cytometry. FIG. 4E shows flow cytometry dot plot data gating strategy to analyze knock-in levels at three tested concentrations of insulin (as in FIG. 4A). Insulin concentrations used: Low: 1 μg/ml, Mid: 5 μg/ml, High: 25 μg/ml. CTR: control.

FIGS. 5A-5G. Insulin pre-treatment enhances T cell metabolism and mitochondria function. FIGS. 5A and 5B show insulin treatment significantly enhances T cell metabolism and glucose uptake both before (FIG. 5A) and 48 h after (FIG. 5B) transfection. FIGS. 5C and 5D show mitochondrial membrane potential (MMP) in insulin treated (Insulin-2N) or untreated (CTR) T cells both before (FIG. 5C) and 48 h after (FIG. 5D) transfection. FIGS. 5E and 5F show mitochondria mass in insulin treated or CTR group T cells. Mitochondria mass 48 h after transfection was significantly higher in T cells treated with insulin (FIG. 5F), while both insulin treated and untreated cultures had comparable mitochondria mass before transfection (FIG. 5E). FIG. 5G shows qPCR analysis for relative expression of Bcl-2L1 transcript levels in CTR and insulin treated groups before transfection. Insulin treatment: treated 24 h pre-transfection with 25 μg/mL insulin. ***P<0.001; **P<0.01; *P<0.05 compared with CTR.

FIGS. 6A-6D. Insulin pre-treatment does not impact FDP characteristics and T cell function. Pre-treatment of T cell cultures with insulin prior to transfection had no impact on the final T cell product phenotype and function. FIGS. 6A and 6B show T cell phenotype data collected from two independent donors. Insulin dosages were used as indicated and in all cases insulin was added 24 h before TFX. Cell samples were collected on day 15, and phenotype ratio was measured by flow cytometry. T stem cell memory (TSCM: CD45RA⁺CD45RO⁻ CD95⁺CD27⁺), T central memory (TCM: CD45RA⁺CD45RO⁺CD95⁺CD27⁺), T cell effector memory (TEM: CD45RA⁻CD45RO CD95⁺CD27⁻), and effector T cells (TE: CD45RACD45RO⁻CD95⁺CD27⁻) phenotypes were measured as indicated. All donors were treated similarly with regards to insulin pre-treatment, electroporation process, and culture conditions and methods. FIG. 6C shows activation marker CD137 expression comparison between insulin pretreated (Insulin-TFX) and control (CTR-TFX) groups. Cell samples were collected on the final day (day 15). Cells were then co-cultured with target peptide (WT1) at indicated concentration for 24 h prior to flow cytometry analysis. FIG. 6D shows cell killing analysis to compare functionality of T cells obtained from insulin pretreated and control groups. T cell samples were collected on the final day (day 15) and co-cultured with target peptide pre-labelled T2 target cells for 20 h at the indicated T-cell:target-cell ratios. Target cell apoptosis levels were measured by calculating annexin V and 7-aminoactinomycin D (7-AAD) double positive populations in T-cell/target-cell cocultured samples vs. the target cell-only culture.

FIGS. 7A and 7B. TCR editing and T cell culture process schematics. FIG. 7A shows an exemplary schematic of exogenic T cell receptor DNA templates used for CRISPR/Cas9 mediated homology directed repair at the TCR-α locus. DNA template including TCR-α variant chain and TCR-β chain have been designed to be inserted into the TCR-α constant (TRAC) locus while endogenous TCR-α (VJ) and TCR-β sites have been disrupted. FIG. 7B shows an exemplary schematic of a 15-day work flow of T cell activation, engineering and cell culture process as described herein. The whole process was performed in chemically defined media.

FIGS. 8A and 8B. Akt and Erk signaling pathways are activated during T cell engineering process and might correlate with expansion rate and viability loss. FIG. 8A shows a Western blot analysis for phosphorylation of Akt and Erk of T cell samples obtained during the T cell engineering process, for two independent donors in presence of both RNP and DNA. FIG. 8B shows T cell viability and expansion profiles for two independent donors during the 15-day T cell engineering and culture process; T cell viability data and cell expansion were measured by NucleoCounter NC-200.

FIGS. 9A-9F. All the tested insulin pre-treatment durations prior to transfection enhanced T cell expansion and TEC levels (three donors each treated with 3 different insulin treatment timepoints). FIGS. 9A-9C show T cell final drug product (FDP) expansion folds for three different donors after a 15-day T cell engineering process. Different pre-TFX insulin treatment timepoints (6 h, 24 h, and 48 h) were tested, calculated based on seeding cell number on day 2, compared with untreated control (CTR). FIGS. 9D-9F show total edited cell (TEC) number in FDP for the engineered T cells shown in FIGS. 9A-9C. The TEC were calculated based on 1 million cell seeding density post transfection per group, rounded up to the whole integers. Total edited cell number was calculated by multiplying the expansion fold with the knock-in percentage. In all cases insulin was used at 25 μg/mL.

FIGS. 10A and 10B. Pre-transfection insulin treatment has the most significant impact on T cell expansion and TEC levels. Insulin was added to activated CD8+T cells 24 h prior to TFX (Pre-TFX), or on day 6 post-TFX (Post-TFX), or both 24 hours pre-TFX and 6 days post-TFX (Pre+Post). FIG. 10A shows T cell final drug product (FDP) expansion folds after a 15-day T cell engineering process using different insulin treatment regimens and concentrations, calculated based on seeding cell number on day 2. FIG. 10B shows total edited cell (TEC) number in FDP. TEC was calculated based on 1 million cell seeding density post transfection per group, rounded up to the whole integers. TEC was calculated by multiplying the expansion fold with the knock-in percentage. Insulin dosages used: NA: 0 μM, Mid: 5 μM, High: 10 μM.

FIGS. 11A-11F. Testing the impact of insulin pre-treatment on T cell metabolism and mitochondria function in a second donor. FIGS. 11A and 11B show T cell metabolism data shows insulin treatment significantly enhances T cell glucose uptake before (FIG. 11A) and 48 h after (FIG. 11B) transfection, as measured by 2-NBDG glucose fluorescence. FIGS. 11C and 11D show mitochondria membrane potential (MMP) was higher in insulin treated T cells (Insulin-2N) when measured 96 hours post transfection compared to control (CTR) (FIG. 11D) while before transfection both groups had comparable MMPs (FIG. 11C). FIGS. 11E and 11F show mitochondria mass in insulin treated T cells, confirming higher mitochondrial mass in insulin treated samples measured 48 hours post transfection (FIG. 11F), while before transfection both groups had comparable mitochondrial mass. ***P<0.001; **P<0.01; *P<0.05 compared with the control group.

FIGS. 12A-12F. Insulin pre-TFX treatment does not impact FDP characteristics and T cell function in either pre-TFX or post-TFX treatment conditions (3 independent donors). Both pre-TFX and post-TFX insulin treated T cells have comparable cell phenotype to that of the control group. FIGS. 12A and 12B show T cell phenotype data collected from Donor 1 for pre-TFX insulin treatment (FIG. 12A) and post-TFX insulin treatment (FIG. 12B). FIGS. 12C and 12D show T cell phenotype data collected from Donor 2 for pre-TFX insulin treatment (FIG. 12C) and post-TFX insulin treatment (FIG. 12D). FIGS. 12E and 12F show T cell phenotype data collected from Donor 3 for pre-TFX insulin treatment (FIG. 12E) and post-TFX insulin treatment (FIG. 12F). Insulin dosages were used as indicated; pre-TFX represent 24 h pre-TFX insulin treatment and post-TFX represent insulin treatment 6 day post TFX. Cell samples were collected on day 15 and different T cell phenotypes were measured by flow cytometry. T stem cell memory (TSCM: CD45RA+CD45RO−CD95+CD27+), T central memory (TCM: CD45RA+CD45RO+CD95+CD27+), T cell effector memory (TEM: CD45RA+CD45RO+CD95+CD27−), and effector T cells (TE: CD45RA+CD45RO−CD95+CD27−) phenotypes were measured as indicated. All donors were treated similarly with regards to insulin pre-treatment, electroporation process, and culture conditions and methods.

FIGS. 13A-13F. Insulin pre-treatment for a shorter duration enhances TCR-engineered T cell expansion and total edited cell number (TECs). FIGS. 13A-13C show T cell final drug product (FDP) expansion folds for three different donors after a 15-day T cell engineering process. Different pre-TFX insulin treatment timepoints (24 h, 6 h, 4 h, 2 h and 30 m) were tested, calculated based on seeding cell number on day 2, compared with untreated control (CTR). FIGS. 13D-13F show total edited cell (TEC) number in FDP for the engineered T cells shown in FIGS. 13A-13C. The TEC were calculated based on 1 million cell seeding density post transfection per group, rounded up to the whole integers. Total edited cell number was calculated by multiplying the expansion fold with the knock-in percentage. In all cases insulin was used at 25 μg/mL.

FIGS. 14A-14C. Insulin treatment enhances T cell total edited cell number (TECs). FIG. 14A shows changes in TECs when T cells were treated with insulin pre-transfection, post-transfection (e.g., during expansion), post-midpoint (e.g., after expansion), or a combination thereof. FIG. 14B shows changes in TECs when T cells were treated prior to transfection with a low (1 μg/ml), medium (mid; 5 μg/ml), or high dose (25 μg/ml) of insulin. FIG. 14C shows changes in TECs when T cells were treated for 30 minutes, 2 hours, 4 hours, 6 hours, or 24 hours with a high dose of insulin prior to transfection. On average, T cell TECs was enhanced by insulin treatment for 2 and 6 hours by 123% and 77%, respectively.

FIGS. 15A-15H. Insulin enhances T cell growth and total edited cell number (TECs) using Xenon transfection. FIGS. 15A-15C show the effects of insulin treatment for 2 h and 6 h on expansion of T cells from Donor 1 (FIG. 15A), Donor 2 (FIG. 15B), and Donor 3 (FIG. 15C). FIGS. 15D-15F show the effects of insulin treatment for 2 h and 6 h on TECs in T cells from Donor 1 (FIG. 15D), Donor 2 (FIG. 15E), and Donor 3 (FIG. 15F). On average, T cell expansion was enhanced by insulin treatment for 2 h and 6 h by 30% and 50%, respectively (FIG. 15G). On average, T cell TECs were enhanced with insulin treatment for 2 h and 6 h by 22% and 47%, respectively (FIG. 15H).

FIGS. 16A-16B. Insulin enhances T cell growth and total edited cell number (TECs) using Xenon transfection in a Good Manufacturing Practices (GMP) scale system. FIG. 16A shows the effects of insulin treatment for 6 h on T cell expansion. FIG. 16B shows the effects of insulin treatment for 6 h on T cell TECs. On average, for 100 million transfected cells, 6 h insulin treatment enhanced T cell expansion and TECs by 27.7% and 23.1%, respectively.

FIGS. 17A-17B. Insulin enhances T cell growth and cell number in diseased patient sample. FIG. 17A shows the results of T cell viability after transfection. FIG. 17B shows the results of T cell number after transfection. Insulin treatment (6 h) partially restored the viability and cell number of a diseased patient after transfection, compared with the non-insulin treated group.

FIGS. 18A-18D. Insulin improved T cell metabolism, mitochondria function and decreased ER stress. FIG. 18A shows the effects of insulin treatment on glucose uptake level. FIG. 18B shows the effects of insulin treatment on mitochondria potential. FIGS. 18C-18D show the effects of insulin treatment on ER stress as measured by ATF4 (FIG. 18C) and IRE1 (FIG. 18D) expression.

FIGS. 19A-19E. Insulin treatment stimulates T cell growth pathway during TCR engineering process. FIGS. 19A-19E shows a Western blot analysis of Akt and Erk and their respective phosphorylated forms in T cells with (Insulin) or without (CTR) insulin treatment pre-transfection. 25 μg/ml insulin used for 24 h treatment. FIG. 19A shows an image of a Western blot gel. FIG. 19B shows the total Akt normalized to Actin expressed in T cells treated with and without insulin. FIG. 19C shows the ratio of phosphorylated Akt (p-Akt) to total Akt in T cells treated with and without insulin. FIG. 19D shows the total Erk normalized to Actin expressed in T cells treated with and without insulin. FIG. 19E shows the ratio of phosphorylated Erk (p-Erk) to total Erk in T cells treated with and without insulin. After insulin treatment, total Akt, total ERK, phosphorylated Akt, and phosphorylated ERK in T cells were all increased after transfection.

FIGS. 20A-20D. Insulin treatment had no impact on gene editing efficiency for either knock-in (KI) or knockout (KO). FIGS. 20A-20B show quantification of gene editing efficiency for knock-in experiments. FIGS. 20C-20D show quantification of gene editing efficiency for knockout experiments.

FIGS. 21A-21C. Insulin treatment had no impact on final cell product (FCP) phenotype. All groups tested keep approximately 97% total memory phenotype (TCM+TSCM %).

FIGS. 22A-22D. Insulin treatment had no impact on final cell product (FCP) function. Results from the CD137 activation assay (FIG. 22A) and IFN-g (FIG. 22B) and TNF-α (FIG. 22C) cytokine expression showed that the insulin treatment and controls respond similarly to target peptide stimulation in all peptide concentration gradients. Results from the T cell to target cell killing assay showed there is comparable killing ability in every E:T ratio between insulin treatment and controls (FIG. 22D).

FIGS. 23A-23B. Negligible insulin residue in T cell FCP culture medium. FIGS. 23A-23B show results from an ELISA analysis of cell culture medium collected on day 12. There was a negligible amount of insulin detected in the culture medium at day 12.

### DETAILED DESCRIPTION

While various embodiments and aspects of the present invention are shown and described herein, it will be obvious to those skilled in the art that such embodiments and aspects are provided by way of example only. Numerous variations, changes, and substitutions will now occur to those skilled in the art without departing from the invention. It should be understood that various alternatives to the embodiments of the invention described herein may be employed in practicing the invention.

The section headings used herein are for organizational purposes only and are not to be construed as limiting the subject matter described. All documents, or portions of documents, cited in the application including, without limitation, patents, patent applications, articles, books, manuals, and treatises are hereby expressly incorporated by reference in their entirety for any purpose.

The abbreviations used herein have their conventional meaning within the chemical and biological arts. The chemical structures and formulae set forth herein are constructed according to the standard rules of chemical valency known in the chemical arts.

Unless defined otherwise, technical and scientific terms used herein have the same meaning as commonly understood by a person of ordinary skill in the art. Any methods, devices and materials similar or equivalent to those described herein can be used in the practice of this invention. The following definitions are provided to facilitate understanding of certain terms used frequently herein and are not meant to limit the scope of the present disclosure.

“Nucleic acid” refers to nucleotides (e.g., deoxyribonucleotides or ribonucleotides) and polymers thereof in either single-, double- or multiple-stranded form, or complements thereof; or nucleosides (e.g., deoxyribonucleosides or ribonucleosides). Examples of polynucleotides contemplated herein include single and double stranded DNA, single and double stranded RNA, and hybrid molecules having mixtures of single and double stranded DNA and RNA. Examples of nucleic acid, e.g. polynucleotides contemplated herein include any types of RNA, e.g., mRNA, siRNA, miRNA, and guide RNA and any types of DNA, e.g., genomic DNA, plasmid DNA, minicircle DNA, linear DNA, and any fragments thereof.

As used herein, the term “gene editing reagent” refers to components required for gene editing tools and may include enzymes, riboproteins, solutions, co-factors and the like. For example, gene editing reagents include one or more components required for Zinc finger nucleases (ZFNs), transcription activator like effector nucleases (TALEN), meganucleases, and clustered regularly interspaced short palindromic repeats system (CRISPR/Cas) gene editing.

As used herein, “zinc finger protein” (ZFP) refers to a chimeric protein including a nuclease domain and a nucleic acid (e.g., DNA) binding domain that is stabilized by zinc. The individual DNA binding domains are typically referred to as “fingers,” such that a zinc finger protein or polypeptide has at least one finger, more typically two fingers, or three fingers, or even four or five fingers, to at least six or more fingers. Each finger typically binds from two to four base pairs of DNA. Each finger may include about 30 amino acids zinc-chelating, DNA-binding region (see, e.g., U.S. Pat. Publ. No. 2012/0329067 A1, the disclosure of which is incorporated herein by reference).

As used herein, “transcription activator-like effectors” (TALEs) refer to proteins composed of more than one TAL repeat and is capable of binding to nucleic acid in a sequence specific manner. TALEs represent a class of DNA binding proteins secreted by plant-pathogenic bacteria of the species, such as Xanthomonas and Ralstonia, via their type III secretion system upon infection of plant cells. Natural TALEs specifically have been shown to bind to plant promoter sequences thereby modulating gene expression and activating effector-specific host genes to facilitate bacterial propagation (Romer, P., et al., Science 318:645-648 (2007); Boch, J., et al., Annu. Rev. Phytopathol. 48:419-436 (2010); Kay, S., et al., Science 318:648-651 (2007); Kay, S., et al., Curr. Opin. Microbiol. 12:37-43 (2009)). The modular structure of TALs allows for combination of the DNA binding domain with effector molecules such as nucleases. In particular, TALE nucleases allow for the development of new genome engineering tools.

Natural TALEs are generally characterized by a central repeat domain and a carboxyl-terminal nuclear localization signal sequence (NLS) and a transcriptional activation domain (AD). The central repeat domain typically consists of a variable amount of between 1.5 and 33.5 amino acid repeats that are usually 33-35 residues in length except for a generally shorter carboxyl-terminal repeat referred to as half-repeat. The repeats are mostly identical but differ in certain hypervariable residues. DNA recognition specificity of TALEs is mediated by hypervariable residues typically at positions 12 and 13 of each repeat—the so-called repeat variable diresidue (RVD) wherein each RVD targets a specific nucleotide in a given DNA sequence. Thus, the sequential order of repeats in a TAL protein tends to correlate with a defined linear order of nucleotides in a given DNA sequence. The underlying RVD code of some naturally occurring TALEs has been identified, allowing prediction of the sequential repeat order required to bind to a given DNA sequence (Boch, J., et al., Science 326:1509-1512 (2009); Moscou, M. J., et al., Science 326:1501 (2009)). Further, TAL effectors generated with new repeat combinations have been shown to bind to target sequences predicted by this code. It has been shown that the target DNA sequence generally start with a 5′ thymine base to be recognized by the TAL protein.

The term “RNA-guided DNA nuclease” or “RNA-guided DNA endonuclease” and the like refer, in the usual and customary sense, to an enzyme that cleave a phosphodiester bond within a DNA polynucleotide chain, wherein the recognition of the phosphodiester bond is facilitated by a separate RNA sequence (for example, a single guide RNA).

The term “Class II CRISPR endonuclease” refers to endonucleases that have similar endonuclease activity as Cas9 and participate in a Class II CRISPR system. An example Class II CRISPR system is the type II CRISPR locus from Streptococcus pyogenes SF370, which contains a cluster of four genes Cas9, Cas1, Cas2, and Csn1, as well as two non-coding RNA elements, tracrRNA and a characteristic array of repetitive sequences (direct repeats) interspaced by short stretches of non-repetitive sequences (spacers, about 30 bp each). The Cpf1 enzyme belongs to a putative type V CRISPR-Cas system. Both type II and type V systems are included in Class II of the CRISPR-Cas system. The C2c1 (“Class 2 candidate 1”) enzyme is a Class II type V-B enzyme. The C2c2 (“Class 2 candidate 2”) enzyme is a Class II type VI-A enzyme. The C2c3 (“Class 2 candidate 3”) enzyme is a Class II type V-C enzyme. Non-limiting exemplary CRISPR associated proteins include Cas1, Cas1B, Cas2, Cas3, Cas4, Cas5, Cas6, Cas7, Cas8, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, Cpf1, C2c1, C2c3, Cas12a, Cas12b, Cas12c, Cas12d, Cas12e, Cas13a, Cas13b, Cas13, nCas9, and Cas-CLOVER. A Class II CRISPR endonuclease can be further modified to be expressed as a fusion protein (e.g. fused with a cytidine or adenine base editor).

A “CRISPR associated protein 9,” “Cas9,” “Csn1” or “Cas9 protein” as referred to herein includes any of the recombinant or naturally-occurring forms of the Cas9 endonuclease or variants or homologs thereof that maintain Cas9 endonuclease enzyme activity (e.g. within at least 50%, 80%, 90%, 95%, 96%, 97%, 98%, 99% or 100% activity compared to Cas9). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas9 protein. In aspects, the Cas9 protein is substantially identical to the protein identified by the UniProt reference number Q99ZW2 or a variant or homolog having substantial identity thereto. In aspects, the Cas9 protein has at least 75% sequence identity to the amino acid sequence of the protein identified by the UniProt reference number Q99ZW2. In aspects, the Cas9 protein has at least 80% sequence identity to the amino acid sequence of the protein identified by the UniProt reference number Q99ZW2. In aspects, the Cas9 protein has at least 85% sequence identity to the amino acid sequence of the protein identified by the UniProt reference number Q99ZW2. In aspects, the Cas9 protein has at least 90% sequence identity to the amino acid sequence of the protein identified by the UniProt reference number Q99ZW2. In aspects, the Cas9 protein has at least 95% sequence identity to the amino acid sequence of the protein identified by the UniProt reference number Q99ZW2.

A “CRISPR-associated endonuclease Cas12a,” “Cas12a,” “Cas12” or “Cas12 protein” as referred to herein includes any of the recombinant or naturally-occurring forms of the Cas12 endonuclease or variants or homologs thereof that maintain Cas12 endonuclease enzyme activity (e.g. within at least 50%, 80%, 90%, 95%, 96%, 97%, 98%, 99% or 100% activity compared to Cas12). In aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas12 protein. In aspects, the Cas12 protein is substantially identical to the protein identified by the UniProt reference number A0Q7Q2 or a variant or homolog having substantial identity thereto.

A “Cfp1” or “Cfp1 protein” as referred to herein includes any of the recombinant or naturally-occurring forms of the Cfp1 (CxxC finger protein 1) endonuclease or variants or homologs thereof that maintain Cfp1 endonuclease enzyme activity (e.g. within at least 50%, 80%, 90%, 95%, 96%, 97%, 98%, 99% or 100% activity compared to Cfp1). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cfp1 protein. In embodiments, the Cfp1 protein is substantially identical to the protein identified by the UniProt reference number Q9POU4 or a variant or homolog having substantial identity thereto.

The term “RNA-guided RNA nuclease” or “RNA-guided RNase” and the like refer, in the usual and customary sense, to an RNA-guided nuclease that targets a specific phosphodiester bond within an RNA polynucleotide, wherein the recognition of the phosphodiester bond is facilitated by a separate polynucleotide sequence (for example, a RNA sequence (e.g., single guide RNA (sgRNA), a guide RNA (gRNA)). Typically, an RNA guided RNase targets single-stranded RNA. In aspects, the RNA-guided RNase is Cas13 (e.g. Cas13a, Cas13b).

A “Cas13a” or “Cas13a protein” as referred to herein includes any of the recombinant or naturally-occurring forms of the Cas13a (CRISPR-associated endoribonuclease Cas13a) endonuclease, also known as CRISPR-associated endoribonuclease C2c2, C2c2, or variants or homologs thereof that maintain Cas13a endonuclease enzyme activity (e.g. within at least 50%, 80%, 90%, 95%, 96%, 97%, 98%, 99% or 100% activity compared to Cas13a). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13a protein. In embodiments, the Cas13a protein is substantially identical to the protein identified by the UniProt reference number C7NBY4 or a variant or homolog having substantial identity thereto.

A “Cas13b” or “Cas13b protein” as referred to herein includes any of the recombinant or naturally-occurring forms of the Cas13b (CRISPR-associated RNA-guided ribonuclease Cas13b) endonuclease, or variants or homologs thereof that maintain Cas13b nuclease enzyme activity (e.g. within at least 50%, 80%, 90%, 95%, 96%, 97%, 98%, 99% or 100% activity compared to Cas13b). In some aspects, the variants or homologs have at least 90%, 95%, 96%, 97%, 98%, 99% or 100% amino acid sequence identity across the whole sequence or a portion of the sequence (e.g. a 50, 100, 150 or 200 continuous amino acid portion) compared to a naturally occurring Cas13b protein. In embodiments, the Cas13b protein is substantially identical to the protein identified by the UniProt reference number A0A8G0P913 or a variant or homolog having substantial identity thereto.

In embodiments, the gene editing reagent includes Cas-CLOVER. In embodiments, Cas-CLOVER includes Clo051 nuclease domain fused with catalytically dead Cas9. See, e.g., U.S. Patent Pub. No. US2021/0107993, and Madison et al., Molecular Therapy Nucleic Acids, Vol. 29, P979-995, Sep. 13, 2022, each of which is incorporated by reference herein in its entirety. In embodiments, the gene editing reagent includes a nickase, e.g., nCas9 (nickase Cas9). Nickases are engineered Cas proteins capable of introducing a single-strand cut with the same specificity as a regular CRISPR/Cas nuclease. See, e.g., PCT Pub. No. WO2014093694, which is incorporated herein by reference in its entirety.

The terms “guide RNA”, and “gRNA”, “single guide RNA”, and “sgRNA” are used interchangeably and refer to the polynucleotide sequence including the crRNA sequence and optionally the tracrRNA sequence. In embodiments, the gRNA includes the crRNA sequence and the tracrRNA sequence. (e.g., “single guide RNA” or “sgRNA”). In embodiments, the gRNA does not include the tracrRNA sequence. The crRNA sequence includes a guide sequence (i.e., “guide” or “spacer”) and a tracr mate sequence (i.e., direct repeat(s)). The term “guide sequence” refers to the sequence that specifies the target site. In general, a tracr mate sequence includes any sequence that has sufficient complementarity with a tracrRNA sequence to promote one or more of: (1) excision of a guide sequence flanked by tracr mate sequences in a cell containing the corresponding tracr sequence; and (2) formation of a complex (e.g., CRISPR complex) at a target sequence, wherein the complex (e.g., CRISPR complex) includes the tracr mate sequence hybridized to the tracr sequence.

In embodiments, the gRNA is a single-stranded ribonucleic acid. In aspects, the gRNA is from about 10 to about 200 nucleic acid residues in length. In aspects, the gRNA is from about 50 to about 150 nucleic acid residues in length. In aspects, the gRNA is from about 80 to about 140 nucleic acid residues in length. In aspects, the gRNA is from about 90 to about 130 nucleic acid residues in length. In aspects, the gRNA is from about 100 to about 120 nucleic acid residues in length.

In general, a guide sequence is any polynucleotide sequence having sufficient complementarity with a target polynucleotide sequence to hybridize with the target sequence and direct sequence-specific binding of a CRISPR complex to the target sequence. In some embodiments, the degree of complementarity between a guide sequence and its corresponding target sequence, when optimally aligned using a suitable alignment algorithm, is about or more than about 50%, 60%, 75%, 80%, 85%, 90%, 95%, 97.5%, 99%, or more. Optimal alignment may be determined with the use of any suitable algorithm for aligning sequences, non-limiting example of which include the Smith-Waterman algorithm, the Needleman-Wunsch algorithm, algorithms based on the Burrows-Wheeler Transform (e.g. the Burrows Wheeler Aligner), ClustalW, Clustal X, BLAST, Novoalign (Novocraft Technologies, ELAND (Illumina, San Diego, Calif.), SOAP (available at soap.genomics.org.cn), and Maq (available at maq.sourceforge.net). In embodiments, a guide sequence is about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length. In embodiments, a guide sequence is less than about 75, 50, 45, 40, 35, 30, 25, 20, 15, 12, or fewer nucleotides in length. The ability of a guide sequence to direct sequence-specific binding of a CRISPR complex to a target sequence may be assessed by any suitable assay. For example, the components of a CRISPR system sufficient to form a CRISPR complex, including the guide sequence to be tested, may be provided to a host cell having the corresponding target sequence, such as by transfection with vectors encoding the components of the CRISPR sequence, followed by an assessment of preferential cleavage within the target sequence, such as by Surveyor assay as described herein. Similarly, cleavage of a target polynucleotide sequence may be evaluated in a test tube by providing the target sequence, components of a CRISPR complex, including the guide sequence to be tested and a control guide sequence different from the test guide sequence, and comparing binding or rate of cleavage at the target sequence between the test and control guide sequence reactions. Other assays are possible, and will occur to those skilled in the art.

As used herein, the term “donor DNA” refers to a single-stranded or double-stranded DNA that can be inserted into the genome of a cell (e.g. a T cell) using genetic modification methods (e.g. CRISPR). For example, the donor DNA may have homology arms that are homologous to a region of a gene where the donor DNA is to be inserted. For example, the donor DNA may form a complex with a Cas protein. In instances, the cell may be transfected with gene editing reagents and the donor DNA. In embodiments, the donor DNA is part of a plasmid, vector, or expression vector that facilitates delivery of the donor DNA into a cell. In embodiments, the donor DNA is part of a circular DNA.

In embodiments, the donor DNA is part of a linear DNA. In embodiments, the donor DNA may include one or more modifications. Nucleic acids (such as donor DNA) used in the methods herein may be modified. For example, the nucleic acids may include known nucleotide analogs or modified backbone residues or linkages, which are synthetic, naturally occurring, and non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, “insulin” refers to the polypeptide hormone that is naturally encoded by the INS gene and is naturally produced in the beta cells of the pancreas. Insulin is also known as “proinsulin”, and also includes any of the recombinant or naturally-occurring forms of insulin, or variants or homologs thereof that maintain insulin activity (e.g. within at least 50%, 80%, 90%, 95%, 96%, 97%, 98%, 99% or 100% activity compared to insulin). In aspects, the variants, analogs, pharmaceutical products, medications, or homologs have at least 90%, 95%, 96%, 97%, 98%, 99% or 100% amino acid sequence identity across the whole sequence or a portion of the sequence compared to a naturally occurring insulin polypeptide. In aspects, the insulin polypeptide is substantially identical to the polypeptide identified by the UniProt reference number P01308 or a variant or homolog having substantial identity thereto.

As used herein, the terms “insulin analog,” “insulin agonist” or “insulin partial agonist” are used interchangeably and refer to any molecule that mimics the activity of the naturally occurring an insulin polypeptide hormone. Where “insulin” is used herein, it is intended to encompass “insulin agonist,” “insulin partial agonist,” or “insulin analog” unless expressly excluded.

The term “gene” means the segment of DNA involved in producing a protein; it includes regions preceding and following the coding region (leader and trailer) as well as intervening sequences (introns) between individual coding segments (exons). The leader, the trailer as well as the introns include regulatory elements that are necessary during the transcription and the translation of a gene. Further, a “protein gene product” is a protein expressed from a particular gene.

The terms “plasmid”, “vector” or “expression vector” refer to a nucleic acid molecule that encodes for genes and/or regulatory elements necessary for the expression of genes. In embodiments, the plasmid, vector, or expression vector is a circular nucleic acid. In embodiments, the plasmid, vector, or expression vector is not a linear nucleic acid. In embodiments, the plasmid, vector, or expression vector is a linear nucleic acid.

As used herein, the term “nanoplasmid” is used to refer to an circular nucleic acid containing at minimum a nucleic acid(s) sequence of interest, a miniature origin of replication (e.g. R6K), and a selectable marker (e.g. a small RNA selectable marker, RNA-OUT). A nanoplasmid contains less than 500 bp of prokaryotic DNA.

As used herein, the term “minicircle” or “mcDNA” is used to refer to an super-coiled, circular plasmid DNA carrying a gene of interest, that is less than 4 kb, and with all prokaryotic vector parts removed.

As used herein, the terms “T cell engineering” or “T cell gene engineering” or the like refer to a type of genetic modification in which DNA is inserted, deleted, modified or replaced at one or more specified locations in the genome of a T cell. Unlike early genetic engineering techniques that randomly insert genetic material into a host genome, T cell engineering targets the genetic modification at site specific locations. Gene editing reagents may be used for T cell engineering to, for example, to a double stranded break at a specific point within a gene or genome where DNA is inserted. A gene editing reagent may include, for example and without limitation, a clustered regularly interspaced short palindromic repeats (CRISPR/Cas) system, ZFN, or TALEN. Thus, an “engineered T cell” is a T cell wherein DNA is inserted, deleted, modified or replaced at one or more specified locations in the T cell genome.

The term “recombinant” when used with reference, e.g., to a virus, cell, nucleic acid, protein, or vector, indicates that the cell (e.g. T cell), nucleic acid, protein or vector, has been modified by the introduction of a heterologous nucleic acid or protein or the alteration of a native nucleic acid or protein, or that the cell is derived from a cell so modified. In instances, recombinant cells express genes that are not found within the native (non-recombinant) form of the cell or express native genes that are otherwise abnormally expressed, under expressed or not expressed at all. Transgenic cells and plants are those that express a heterologous gene or coding sequence, typically as a result of recombinant methods.

The term “heterologous” when used with reference to portions of a nucleic acid indicates that the nucleic acid includes two or more subsequences that are not found in the same relationship to each other in nature. For instance, the nucleic acid may be recombinantly produced, having two or more sequences from unrelated genes arranged to make a new functional nucleic acid, e.g., a promoter from one source and a coding region from another source. Similarly, a heterologous protein indicates that the protein includes two or more subsequences that are not found in the same relationship to each other in nature (e.g., a fusion protein).

The term “exogenous” refers to a molecule or substance (e.g., a compound, nucleic acid or protein) that originates from outside a given cell or organism. For example, an “exogenous promoter” as referred to herein is a promoter that does not originate from the cell or organism it is expressed by. Conversely, the term “endogenous” or “endogenous promoter” refers to a molecule or substance that is native to, or originates within, a given cell or organism.

The term “isolated”, when applied to a nucleic acid or protein, denotes that the nucleic acid or protein is essentially free of other cellular components with which it is associated in the natural state. It can be, for example, in a homogeneous state and may be in either a dry or aqueous solution. Purity and homogeneity are typically determined using analytical chemistry techniques such as polyacrylamide gel electrophoresis or high performance liquid chromatography. A nucleic acid that is the predominant species present in a preparation is substantially purified.

As used herein, the term “electroporation”, “electropermeabilization”, and “electrotransfer” are used in accordance with its plain ordinary meaning and refer to a technique in which an electrical field is applied to cells in order to increase the permeability of the cell membrane, allowing chemicals, drugs, proteins, or nucleic acids, or combinations thereof to be introduced into the cell.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetofection and electroporation. In some embodiments, the nucleic acid molecules are introduced into a cell using electroporation following standard procedures well known in the art. For viral-based methods of transfection any useful viral vector (e.g. adenovirus vector) may be used in the methods described herein. Examples for viral vectors include, but are not limited to retroviral, adenoviral, lentiviral and adeno-associated viral vectors. In some embodiments, the nucleic acid molecules are introduced into a cell using an adenoviral vector following standard procedures well known in the art. The terms “transfection” or “transduction” also refer to introducing proteins into a cell from the external environment. In embodiments, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

“Transduce” or “transduction” are used according to their plain ordinary meanings and refer to the process by which one or more foreign nucleic acids (i.e. DNA not naturally found in the cell) are introduced into a cell. Transduction may occur by introduction of a virus or viral vector (e.g. adenovirus vector) into the cell.

The word “expression” or “expressed” as used herein in reference to a gene means the transcriptional and/or translational product of that gene (e.g. a TCR-alpha, TCR-beta, etc.). The level of expression of a DNA molecule in a cell may be determined on the basis of either the amount of corresponding mRNA that is present within the cell or the amount of protein encoded by that DNA produced by the cell. The level of expression of nucleic acid molecules may be detected by standard methods, including PCR or Northern blot methods well known in the art. See, e.g., Sambrook et al., 1989 Molecular Cloning: A Laboratory Manual, 18.1-18.88.

“Contacting” is used in accordance with its plain ordinary meaning and refers to the process of allowing at least two distinct species (e.g. chemical compounds including biomolecules or cells) to become sufficiently proximal to react, interact or physically touch. The two species may be, for example, insulin or insulin analog and a T cell. In embodiments contacting includes, for example, allowing insulin or insulin analog described herein to physically touch a T cell. In embodiments, the contacting may result in delivery of a compound into a cell. For example, the contacting may result in delivery of insulin or insulin analog into a cell. In embodiments, the contacting may result in delivery of a nucleic acid into the cell. In embodiments, “contacting” or “contacted” includes culturing T cells in the presence of a species, e.g., insulin or insulin analog.

A “control” or “standard control” refers to a sample, measurement, or value that serves as a reference, usually a known reference, for comparison to a test sample, measurement, or value. For example, a standard control may be an engineered T cell made without contacting (e.g., culturing) a T cell with one or more insulin or insulin analogs as provided herein including embodiments thereof. In embodiments, a standard control may be a population of engineered T cells made without contacting (e.g., culturing) a population of T cells with one or more insulin or insulin analogs as provided herein including embodiments thereof. Thus, the standard control may be engineered T cell(s) made by contacting a T cell with a nucleic acid without one or more insulin or insulin analogs. The standard control may be a population of engineered T cells made by contacting a population of T cells with a nucleic acid without one or more insulin or insulin analogs. Controls also are valuable for determining the significance of data. For example, if values for a given parameter are widely variant in controls, variation in test samples will not be considered as significant. One of skill will recognize that standard controls can be designed for assessment of any number of parameters (e.g. cell viability, cell expansion, total edited cell number, gene editing efficiency, etc.). One of skill in the art will understand which standard controls are most appropriate in a given situation and be able to analyze data based on comparisons to standard control values.

“T cells” or “T lymphocytes” as used herein are a type of lymphocyte (a subtype of white blood cell) that plays a central role in cell-mediated immunity. They can be distinguished from other lymphocytes, such as B cells and natural killer cells, by the presence of a T-cell receptor on the cell surface. T cells include, for example, natural killer T (NKT) cells, cytotoxic T lymphocytes (CTLs), regulatory T (Treg) cells, and T helper cells. Different types of T cells can be distinguished by use of T cell detection agents.

As defined herein, the term “inhibition”, “inhibit”, “inhibiting” and the like in reference to cell proliferation means negatively affecting (e.g., decreasing proliferation) or killing the cell. In some embodiments, inhibition refers to reduction of a disease or symptoms of disease (e.g., cancer, cancer cell proliferation). In embodiments, “inhibitor” is a compound or protein that inhibits a receptor or another protein, e.g., by binding, partially or totally blocking, decreasing, preventing, delaying, inactivating, desensitizing, or down-regulating activity (e.g., a receptor activity or a protein activity).

The terms “disease” or “condition” refer to a state of being or health status of a patient or subject capable of being treated with the compounds or methods provided herein. The disease may be cancer. In some further instances, “cancer” refers to human cancers. In embodiments, the cancer is lymphoma, melanoma, or leukemia.

“Patient”, “subject” or “subject in need thereof” refers to a living organism suffering from or prone to a disease (e.g. cancer, etc.) or condition that can be treated by administration of a composition or pharmaceutical composition as provided herein. Non-limiting examples include humans, other mammals, bovines, rats, mice, dogs, monkeys, goat, sheep, cows, deer, and other non-mammalian animals. In some embodiments, a subject is human.

As used herein, the term “cancer” refers to all types of cancer, neoplasm or malignant tumors found in mammals (e.g. humans), including leukemias, lymphomas, carcinomas and sarcomas. Exemplary cancers that may be treated with a compound or method provided herein include brain cancer, glioma, glioblastoma, neuroblastoma, prostate cancer, colorectal cancer, pancreatic cancer, Medulloblastoma, melanoma, cervical cancer, gastric cancer, ovarian cancer, lung cancer, cancer of the head, Hodgkin's Disease, and Non-Hodgkin's Lymphomas. Exemplary cancers that may be treated with a compound or method provided herein include cancer of the thyroid, endocrine system, brain, breast, cervix, colon, head & neck, liver, kidney, lung, ovary, pancreas, rectum, stomach, and uterus. Additional examples include, thyroid carcinoma, cholangiocarcinoma, pancreatic adenocarcinoma, skin cutaneous melanoma, colon adenocarcinoma, rectum adenocarcinoma, stomach adenocarcinoma, esophageal carcinoma, head and neck squamous cell carcinoma, breast invasive carcinoma, lung adenocarcinoma, lung squamous cell carcinoma, non-small cell lung carcinoma, mesothelioma, multiple myeloma, neuroblastoma, glioma, glioblastoma multiforme, ovarian cancer, rhabdomyosarcoma, primary thrombocytosis, primary macroglobulinemia, primary brain tumors, malignant pancreatic insulanoma, malignant carcinoid, urinary bladder cancer, premalignant skin lesions, testicular cancer, thyroid cancer, neuroblastoma, esophageal cancer, genitourinary tract cancer, malignant hypercalcemia, endometrial cancer, adrenal cortical cancer, neoplasms of the endocrine or exocrine pancreas, medullary thyroid cancer, medullary thyroid carcinoma, melanoma, colorectal cancer, papillary thyroid cancer, hepatocellular carcinoma, or prostate cancer.

As used herein, “treating” or “treatment of” a condition, disease or disorder or symptoms associated with a condition, disease or disorder refers to an approach for obtaining beneficial or desired results, including clinical results. Beneficial or desired clinical results can include, but are not limited to, alleviation or amelioration of one or more symptoms or conditions, diminishment of extent of condition, disorder or disease, stabilization of the state of condition, disorder or disease, prevention of spread of condition, disorder or disease, delay or slowing of condition, disorder or disease progression, delay or slowing of condition, disorder or disease onset, amelioration or palliation of the condition, disorder or disease state, and remission, whether partial or total. “Treating” can also mean prolonging survival of a subject beyond that expected in the absence of treatment. “Treating” can also mean inhibiting the progression of the condition, disorder or disease, slowing the progression of the condition, disorder or disease temporarily, although in some instances, it involves halting the progression of the condition, disorder or disease permanently.

The terms “dose” and “dosage” are used interchangeably herein. A dose refers to the amount of active ingredient given to an individual at each administration. The dose will vary depending on a number of factors, including the range of normal doses for a given therapy, frequency of administration; size and tolerance of the individual; severity of the condition; risk of side effects; and the route of administration. One of skill will recognize that the dose can be modified depending on the above factors or based on therapeutic progress.

By “therapeutically effective dose or amount” as used herein is meant a dose that produces effects for which it is administered (e.g. treating or preventing a disease). The exact dose and formulation will depend on the purpose of the treatment, and will be ascertainable by one skilled in the art using known techniques (see, e.g., Lieberman, Pharmaceutical Dosage Forms (vols. 1-3, 1992); Lloyd, The Art, Science and Technology of Pharmaceutical Compounding (1999); Remington: The Science and Practice of Pharmacy, 20th Edition, Gennaro, Editor (2003), and Pickar, Dosage Calculations (1999)). For example, for the given parameter, a therapeutically effective amount will show an increase or decrease of at least 5%, 10%, 15%, 20%, 25%, 40%, 50%, 60%, 75%, 80%, 90%, or at least 100%. Therapeutic efficacy can also be expressed as “-fold” increase or decrease. For example, a therapeutically effective amount can have at least a 1.2-fold, 1.5-fold, 2-fold, 5-fold, or more effect over a standard control. A therapeutically effective dose or amount may ameliorate one or more symptoms of a disease.

As used herein, the term “administering” is used in accordance with its plain and ordinary meaning and includes any administration appropriate for cell therapy. For example, administration may be parenteral. Parenteral administration includes, e.g., intravenous, intramuscular, intra-arteriole, intradermal, subcutaneous, intraperitoneal, intraventricular, and intracranial. In embodiments, administration is intravenous.

### Methods

Provided herein, inter alia, are methods for engineering a T cell, including contacting the T cell with insulin, an insulin analog, an insulin agonist and/or an insulin partial agonist. The methods provided herein allow formation of an engineered T cell while improving viability and growth of T cells compared to previously known methods of engineering T cells. For example, the methods provided herein are contemplated to be effective for improving growth and viability by activating parallel growth signaling pathways.

In an aspect, provided herein, is a method of editing an endogenous gene in a population of T cells, the method including: contacting the population of T cells with a gene editing reagent or a polynucleotide encoding a gene editing reagent under conditions to allow the polynucleotide or gene editing reagent to enter the cell, and culturing the population of T cells in the presence of one or more of, insulin, an insulin analog, an insulin agonist and/or an insulin partial agonist before and/or during, and/or after the contacting step to obtain an engineered population of T cells.

For the methods provided herein, in embodiments, further including contacting the population of T cells with a donor DNA. In embodiments, the polynucleotide encoding the gene editing reagent includes: single-stranded DNA, double-stranded DNA, a linear DNA strand, a plasmid, a nanoplasmid, or a minicircle. In embodiments, the polynucleotide encoding the gene editing reagent includes single-stranded DNA. In embodiments, the polynucleotide encoding the gene editing reagent includes double-stranded DNA. In embodiments, the polynucleotide encoding the gene editing reagent includes a linear DNA strand. In embodiments, the polynucleotide encoding the gene editing reagent includes a plasmid. In embodiments, the polynucleotide encoding the gene editing reagent includes a nanoplasmid. In embodiments, the polynucleotide encoding the gene editing reagent includes a minicircle.

In embodiments, the polynucleotide encoding the gene editing reagent includes a plasmid including a plasmid backbone and a polynucleotide sequence encoding the gene editing reagent. In embodiments, the plasmid further includes the donor DNA. In embodiments, the donor DNA sequence includes a polynucleotide encoding a gene product. In embodiments, the population of T cells was obtained from a subject.

In embodiments, the gene product sequence includes a chimeric antigen receptor (CAR), a T cell receptor (TCR), a human leukocyte antigen (HLA), or an alloimmune defense receptor (ADR), or a subunit thereof. In embodiments, the gene product sequence includes a chimeric antigen receptor (CAR). In embodiments, the gene product sequence includes a T cell receptor (TCR). In embodiments, the gene product sequence includes a human leukocyte antigen (HLA). In embodiments, the gene product sequence includes an alloimmune defense receptor (ADR).

In embodiments, the TCR sequence includes an exogenous TCR-beta subunit or fragment thereof and/or an exogenous TCR-alpha subunit or fragment thereof, or a chimeric antigen receptor and/or subunit thereof. In embodiments, the TCR sequence includes an exogenous TCR-beta subunit or fragment thereof and an exogenous TCR-alpha subunit or fragment thereof. In embodiments, the TCR sequence includes an exogenous TCR-beta subunit or fragment thereof or an exogenous TCR-alpha subunit or fragment thereof. In embodiments, the TCR sequence includes an exogenous TCR-beta subunit or fragment thereof. In embodiments, the TCR sequence includes an exogenous TCR-alpha subunit or fragment thereof. In embodiments, the TCR sequence includes a chimeric antigen receptor and subunit thereof. In embodiments, the TCR sequence includes a chimeric antigen receptor or subunit thereof. In embodiments, the TCR sequence is inserted in a TRAC or TRBC locus. In embodiments, the TCR sequence is inserted in a TRAC locus. In embodiments, the TCR sequence is inserted in a TRBC locus.

In embodiments, contacting the population of T cells with the gene editing reagent or polynucleotide encoding the gene reagent includes transfecting the population of T cells with the gene editing reagent or polynucleotide encoding the gene editing reagent. In embodiments, contacting the population of T cells with the gene editing reagent includes transfecting the population of T cells with the gene editing reagent. In embodiments, contacting the population of T cells with the polynucleotide encoding the gene reagent includes transfecting the population of T cells with the polynucleotide encoding the gene editing reagent. In embodiments, the transfecting includes electroporation. In embodiments, the transfecting includes nucleofection. In embodiments, the transfecting includes lipid transfection.

In instances, delivery of the polynucleotide into the T cell may be facilitated by a delivery vehicle. The delivery vehicle may facilitate interaction of the polynucleotide and the T cell membrane, thereby allowing entry of the polynucleotide into the T cell. In one example, the polynucleotide may be encapsulated in the delivery vehicle. In another example, the polynucleotide may be non-covalently associated with the delivery vehicle. Thus, in embodiments, the polynucleotide is associated with a delivery vehicle. In embodiments, the delivery vehicle is a lipid particle or a nanoparticle. In embodiments, the delivery vehicle is a lipid particle. In embodiments, the delivery vehicle is a nanoparticle. In embodiments, the delivery vehicle is a liposome or a lipid nanoparticle.

For the methods provided herein, in embodiments, the T cell is a primary T cell. “Primary T cell” is used in accordance to its ordinary meaning in the biological arts and refers to a T cell that is directly expanded from a T cell extracted from a subject. A “secondary T cell” is therefore a T cell expanded from the primary T cell. For example, a secondary T cell is a T cell expanded via the primary T cell culture.

For the methods provided herein, the gene editing reagent and the polynucleotide may be delivered into the T cell using a variety of methods known in the art, including but not limited to electroporation and transfection methods. In embodiments, contacting the T cell with the gene editing reagent includes transfecting the T cell with the gene editing reagent. In embodiments, contacting the T cell with the polynucleotide includes transfecting the T cell with the polynucleotide.

As described above, the insulin is a naturally occurring insulin polypeptide. In some embodiments, the insulin has a propeptide configuration. In some embodiments, the insulin is mature, native disulfide bonded A and B chain insulin. In some embodiments, the insulin is a sequence variant of a naturally-occurring or wild-type insulin sequence. In some embodiments, the insulin is purified from animal sources. In some embodiments, the insulin is of shark origin. In some embodiments, the insulin is of fish origin. In some embodiments, the insulin is of mammalian origin. In some embodiments, the insulin is a recombinantly produced. In some embodiments, the recombinantly-produced insulin is a mammalian polypeptide sequence. In some embodiments, the recombinantly-produced insulin is the porcine polypeptide sequence. In some embodiments, the recombinantly-produced insulin is the human polypeptide sequence. In some embodiments, the human sequence is identical to the full sequence of P01308 (UniProtKB). In some embodiments, the human sequence is identical to a portion of P01308 (UniProtKB). In some embodiments, the human sequence includes the A and B chain of P01308 (UniProtKB). In some embodiments, the insulin is a pharmaceutical product. In some embodiments, the insulin is a commercial product not available for use as a pharmaceutical.

In embodiments, the insulin is an insulin analog (agonist). In embodiments, the insulin analog produces the same effect as naturally occurring insulin when applied to a T cell. In embodiments, the insulin analog produces a substantially similar effect as naturally occurring insulin when applied to a T cell. In embodiments, the insulin analog is long-acting or short-acting. In embodiments, the long-acting insulin analog includes detemir, glargine, degludec, or combinations thereof. In embodiments, the short-acting insulin analog includes aspart, lispro, glulisine, or combinations thereof. In embodiments, the one or more insulin analog includes detemir. In embodiments, the one or more insulin analog includes glargine. In embodiments, the one or more insulin analog includes degludec. In embodiments, the one or more insulin analog includes aspart. In embodiments, the one or more insulin analog includes lispro. In embodiments, the one or more insulin analog includes glulisine.

In embodiments, the insulin analog is detemir. In embodiments, the insulin analog is glargine. In embodiments, the insulin analog is degludec. In embodiments, the insulin analog is aspart. In embodiments, the insulin analog is lispro. In embodiments, the insulin analog is glulisine.

Applicant has surprisingly discovered that treatment of the T cell with insulin prior to contacting the T cell with the polynucleotide or in the presence of the polynucleotide improves effectiveness of T cell engineering. In embodiments, the T cell and the polynucleotide are contacted in the presence of insulin. For example, the T cell may be transfected with the polynucleotide in the presence of insulin. In another example, the T cell may be electroporated with the polynucleotide in the presence of insulin. In embodiments, the T cell is contacted sequentially with the polynucleotide and insulin. In embodiments, T cell is contacted with insulin prior to the polynucleotide. For example, the insulin may be added to the T cell culture prior to transfecting the T cells with the polynucleotide. In embodiments, T cell is contacted with insulin after the polynucleotide. For example, the insulin may be added to the T cell culture after transfecting the T cells with the polynucleotide.

In embodiments, the population of T cells is cultured in the presence of insulin, an insulin analog, an insulin agonist and/or an insulin partial agonist prior to the contacting step. In embodiments, the population of T cells is cultured in the presence of insulin, an insulin analog, an insulin agonist and an insulin partial agonist prior to the contacting step. In embodiments, the population of T cells is cultured in the presence of insulin, an insulin analog, an insulin agonist or an insulin partial agonist prior to the contacting step. In embodiments, the population of T cells is cultured in the presence of insulin prior to the contacting step. In embodiments, the population of T cells is cultured in the presence of an insulin analog prior to the contacting step. In embodiments, the population of T cells is cultured in the presence of an insulin agonist prior to the contacting step. In embodiments, the population of T cells is cultured in the presence of an insulin partial agonist prior to the contacting step. In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step. In embodiments, including culturing the population of T cells in the presence of insulin, an insulin analog, an insulin agonist and/or an insulin partial agonist for 48 hours prior to the contacting step. In embodiments, including culturing the population of T cells in the presence of insulin, an insulin analog, an insulin agonist and/or an insulin partial agonist for 24 hours prior to the contacting step. In embodiments, including culturing the population of T cells in the presence of insulin, an insulin analog, an insulin agonist and/or an insulin partial agonist for 12 hours prior to the contacting step. In embodiments, including culturing the population of T cells in the presence of insulin, an insulin analog, an insulin agonist and/or an insulin partial agonist for 6 hours prior to the contacting step. In embodiments, including culturing the population of T cells in the presence of insulin, an insulin analog, an insulin agonist and/or an insulin partial agonist for 4 hours prior to the contacting step. In embodiments, including culturing the population of T cells in the presence of insulin, an insulin analog, an insulin agonist and/or an insulin partial agonist for 2 prior to the contacting step. In embodiments, including culturing the population of T cells in the presence of insulin, an insulin analog, an insulin agonist and/or an insulin partial agonist for 1 hour prior to the contacting step. In embodiments, including culturing the population of T cells in the presence of insulin, an insulin analog, an insulin agonist and/or an insulin partial agonist for 30 minutes prior to the contacting step.

In embodiments, the population of T cells is cultured in the presence of insulin, an insulin analog, an insulin agonist and/or an insulin partial agonist after the contacting step. In embodiments, the population of T cells is cultured in the presence of insulin, an insulin analog, an insulin agonist and an insulin partial agonist after the contacting step. In embodiments, the population of T cells is cultured in the presence of insulin, an insulin analog, an insulin agonist or an insulin partial agonist after the contacting step. In embodiments, the population of T cells is cultured in the presence of insulin after the contacting step. In embodiments, the population of T cells is cultured in the presence of an insulin analog after the contacting step. In embodiments, the population of T cells is cultured in the presence of an insulin agonist after the contacting step. In embodiments, the population of T cells is cultured in the presence of an insulin partial agonist after the contacting step. In embodiments, including culturing the population of T cells in the presence of insulin, an insulin analog, an insulin agonist and/or an insulin partial agonist for 48 hours after the contacting step. In embodiments, including culturing the population of T cells in the presence of insulin, an insulin analog, an insulin agonist and/or an insulin partial agonist for 24 hours after the contacting step. In embodiments, including culturing the population of T cells in the presence of insulin, an insulin analog, an insulin agonist and/or an insulin partial agonist for 12 hours after the contacting step. In embodiments, including culturing the population of T cells in the presence of insulin, an insulin analog, an insulin agonist and/or an insulin partial agonist for 6 hours after the contacting step. In embodiments, including culturing the population of T cells in the presence of insulin, an insulin analog, an insulin agonist and/or an insulin partial agonist for 4 hours after the contacting step. In embodiments, including culturing the population of T cells in the presence of insulin, an insulin analog, an insulin agonist and/or an insulin partial agonist for 2 hours after the contacting step. In embodiments, including culturing the population of T cells in the presence of insulin, an insulin analog, an insulin agonist and/or an insulin partial agonist for 1 hour after the contacting step. In embodiments, including culturing the population of T cells in the presence of insulin, an insulin analog, an insulin agonist and/or an insulin partial agonist for 30 minutes after the contacting step.

In embodiments, the population of T cells is cultured in the presence of insulin, an insulin analog, an insulin agonist and/or an insulin partial agonist prior to and after the contacting step. In embodiments, the population of T cells is cultured in the presence of insulin, an insulin analog, an insulin agonist and an insulin partial agonist prior to and after the contacting step. In embodiments, the population of T cells is cultured in the presence of insulin, an insulin analog, an insulin agonist or an insulin partial agonist prior to and after the contacting step. In embodiments, the population of T cells is cultured in the presence of insulin prior to and after the contacting step. In embodiments, the population of T cells is cultured in the presence of an insulin analog prior to and after the contacting step. In embodiments, the population of T cells is cultured in the presence of an insulin agonist prior to and after the contacting step. In embodiments, the population of T cells is cultured in the presence of an insulin partial agonist prior to and after the contacting step.

In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step. In embodiments, including culturing the population of T cells in the presence of insulin, an insulin analog, an insulin agonist and/or an insulin partial agonist for 48 hours prior to the contacting step. In embodiments, including culturing the population of T cells in the presence of insulin, an insulin analog, an insulin agonist and/or an insulin partial agonist for 24 hours prior to the contacting step. In embodiments, including culturing the population of T cells in the presence of insulin, an insulin analog, an insulin agonist and/or an insulin partial agonist for 12 hours prior to the contacting step. In embodiments, including culturing the population of T cells in the presence of insulin, an insulin analog, an insulin agonist and/or an insulin partial agonist for 6 hours prior to the contacting step. In embodiments, including culturing the population of T cells in the presence of insulin, an insulin analog, an insulin agonist and/or an insulin partial agonist for 4 hours prior to the contacting step. In embodiments, including culturing the population of T cells in the presence of insulin, an insulin analog, an insulin agonist and/or an insulin partial agonist for 2 prior to the contacting step. In embodiments, including culturing the population of T cells in the presence of insulin, an insulin analog, an insulin agonist and/or an insulin partial agonist for 1 hour prior to the contacting step. In embodiments, including culturing the population of T cells in the presence of insulin, an insulin analog, an insulin agonist and/or an insulin partial agonist for 30 minutes prior to the contacting step.

In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes after the contacting step. In embodiments, including culturing the population of T cells in the presence of insulin, an insulin analog, an insulin agonist and/or an insulin partial agonist for 48 hours after the contacting step. In embodiments, including culturing the population of T cells in the presence of insulin, an insulin analog, an insulin agonist and/or an insulin partial agonist for 24 hours after the contacting step. In embodiments, including culturing the population of T cells in the presence of insulin, an insulin analog, an insulin agonist and/or an insulin partial agonist for 12 hours after the contacting step. In embodiments, including culturing the population of T cells in the presence of insulin, an insulin analog, an insulin agonist and/or an insulin partial agonist for 6 hours after the contacting step. In embodiments, including culturing the population of T cells in the presence of insulin, an insulin analog, an insulin agonist and/or an insulin partial agonist for 4 hours after the contacting step. In embodiments, including culturing the population of T cells in the presence of insulin, an insulin analog, an insulin agonist and/or an insulin partial agonist for 2 hours after the contacting step. In embodiments, including culturing the population of T cells in the presence of insulin, an insulin analog, an insulin agonist and/or an insulin partial agonist for 1 hour after the contacting step. In embodiments, including culturing the population of T cells in the presence of insulin, an insulin analog, an insulin agonist and/or an insulin partial agonist for 30 minutes after the contacting step.

In embodiments, the insulin, an insulin analog, an insulin agonist and/or an insulin partial agonist is administered at a concentration of about 1 μg/ml to about 50 μg/ml. In embodiments, the insulin, an insulin analog, an insulin agonist and/or an insulin partial agonist is administered at a concentration of about 2 μg/ml to about 50 μg/ml. In embodiments, the insulin, an insulin analog, an insulin agonist and/or an insulin partial agonist is administered at a concentration of about 3 μg/ml to about 50 μg/ml. In embodiments, the insulin, an insulin analog, an insulin agonist and/or an insulin partial agonist is administered at a concentration of about 4 μg/ml to about 50 μg/ml. In embodiments, the insulin, an insulin analog, an insulin agonist and/or an insulin partial agonist is administered at a concentration of about 5 μg/ml to about 50 μg/ml. In embodiments, the insulin, an insulin analog, an insulin agonist and/or an insulin partial agonist is administered at a concentration of about 6 μg/ml to about 50 μg/ml. In embodiments, the insulin, an insulin analog, an insulin agonist and/or an insulin partial agonist is administered at a concentration of about 7 μg/ml to about 50 μg/ml. In embodiments, the insulin, an insulin analog, an insulin agonist and/or an insulin partial agonist is administered at a concentration of about 8 μg/ml to about 50 μg/ml. In embodiments, the insulin, an insulin analog, an insulin agonist and/or an insulin partial agonist is administered at a concentration of about 9 μg/ml to about 50 μg/ml. In embodiments, the insulin, an insulin analog, an insulin agonist and/or an insulin partial agonist is administered at a concentration of about 10 μg/ml to about 50 μg/ml. In embodiments, the insulin, an insulin analog, an insulin agonist and/or an insulin partial agonist is administered at a concentration of about 15 μg/ml to about 50 μg/ml. In embodiments, the insulin, an insulin analog, an insulin agonist and/or an insulin partial agonist is administered at a concentration of about 20 μg/ml to about 50 μg/ml. In embodiments, the insulin, an insulin analog, an insulin agonist and/or an insulin partial agonist is administered at a concentration of about 25 μg/ml to about 50 μg/ml. In embodiments, the insulin, an insulin analog, an insulin agonist and/or an insulin partial agonist is administered at a concentration of about 30 μg/ml to about 50 μg/ml. In embodiments, the insulin, an insulin analog, an insulin agonist and/or an insulin partial agonist is administered at a concentration of about 35 μg/ml to about 50 μg/ml. In embodiments, the insulin, an insulin analog, an insulin agonist and/or an insulin partial agonist is administered at a concentration of about 40 μg/ml to about 50 μg/ml. In embodiments, the insulin, an insulin analog, an insulin agonist and/or an insulin partial agonist is administered at a concentration of about 45 μg/ml to about 50 μg/ml.

In embodiments, the insulin, an insulin analog, an insulin agonist and/or an insulin partial agonist is administered at a concentration of about 1 μg/ml to about 45 μg/ml. In embodiments, the insulin, an insulin analog, an insulin agonist and/or an insulin partial agonist is administered at a concentration of about 1 μg/ml to about 40 μg/ml. In embodiments, the insulin, an insulin analog, an insulin agonist and/or an insulin partial agonist is administered at a concentration of about 1 μg/ml to about 35 μg/ml. In embodiments, the insulin, an insulin analog, an insulin agonist and/or an insulin partial agonist is administered at a concentration of about 1 μg/ml to about 30 μg/ml. In embodiments, the insulin, an insulin analog, an insulin agonist and/or an insulin partial agonist is administered at a concentration of about 1 μg/ml to about 25 μg/ml. In embodiments, the insulin, an insulin analog, an insulin agonist and/or an insulin partial agonist is administered at a concentration of about 1 μg/ml to about 20 μg/ml. In embodiments, the insulin, an insulin analog, an insulin agonist and/or an insulin partial agonist is administered at a concentration of about 1 μg/ml to about 15 μg/ml. In embodiments, the insulin, an insulin analog, an insulin agonist and/or an insulin partial agonist is administered at a concentration of about 1 μg/ml to about 10 μg/ml. In embodiments, the insulin, an insulin analog, an insulin agonist and/or an insulin partial agonist is administered at a concentration of about 1 μg/ml to about 9 μg/ml. In embodiments, the insulin, an insulin analog, an insulin agonist and/or an insulin partial agonist is administered at a concentration of about 1 μg/ml to about 8 μg/ml. In embodiments, the insulin, an insulin analog, an insulin agonist and/or an insulin partial agonist is administered at a concentration of about 1 μg/ml to about 7 μg/ml. In embodiments, the insulin, an insulin analog, an insulin agonist and/or an insulin partial agonist is administered at a concentration of about 1 μg/ml to about 6 μg/ml. In embodiments, the insulin, an insulin analog, an insulin agonist and/or an insulin partial agonist is administered at a concentration of about 1 μg/ml to about 5 μg/ml. In embodiments, the insulin, an insulin analog, an insulin agonist and/or an insulin partial agonist is administered at a concentration of about 1 μg/ml to about 4 μg/ml. In embodiments, the insulin, an insulin analog, an insulin agonist and/or an insulin partial agonist is administered at a concentration of about 1 μg/ml to about 3 μg/ml. In embodiments, the insulin, an insulin analog, an insulin agonist and/or an insulin partial agonist is administered at a concentration of about 1 μg/ml to about 2 μg/ml.

In embodiments, the insulin, an insulin analog, an insulin agonist and/or an insulin partial agonist is administered at a concentration of 1 μg/ml to 50 μg/ml. In embodiments, the insulin, an insulin analog, an insulin agonist and/or an insulin partial agonist is administered at a concentration of 2 μg/ml to 50 μg/ml. In embodiments, the insulin, an insulin analog, an insulin agonist and/or an insulin partial agonist is administered at a concentration of 3 μg/ml to 50 μg/ml. In embodiments, the insulin, an insulin analog, an insulin agonist and/or an insulin partial agonist is administered at a concentration of 4 μg/ml to 50 μg/ml. In embodiments, the insulin, an insulin analog, an insulin agonist and/or an insulin partial agonist is administered at a concentration of 5 μg/ml to 50 μg/ml. In embodiments, the insulin, an insulin analog, an insulin agonist and/or an insulin partial agonist is administered at a concentration of 6 μg/ml to 50 μg/ml. In embodiments, the insulin, an insulin analog, an insulin agonist and/or an insulin partial agonist is administered at a concentration of 7 μg/ml to 50 μg/ml. In embodiments, the insulin, an insulin analog, an insulin agonist and/or an insulin partial agonist is administered at a concentration of 8 μg/ml to 50 μg/ml. In embodiments, the insulin, an insulin analog, an insulin agonist and/or an insulin partial agonist is administered at a concentration of 9 μg/ml to 50 μg/ml. In embodiments, the insulin, an insulin analog, an insulin agonist and/or an insulin partial agonist is administered at a concentration of 10 μg/ml to 50 μg/ml. In embodiments, the insulin, an insulin analog, an insulin agonist and/or an insulin partial agonist is administered at a concentration of 15 μg/ml to 50 μg/ml. In embodiments, the insulin, an insulin analog, an insulin agonist and/or an insulin partial agonist is administered at a concentration of 20 μg/ml to 50 μg/ml. In embodiments, the insulin, an insulin analog, an insulin agonist and/or an insulin partial agonist is administered at a concentration of 25 μg/ml to 50 μg/ml. In embodiments, the insulin, an insulin analog, an insulin agonist and/or an insulin partial agonist is administered at a concentration of 30 μg/ml to 50 μg/ml. In embodiments, the insulin, an insulin analog, an insulin agonist and/or an insulin partial agonist is administered at a concentration of 35 μg/ml to 50 μg/ml. In embodiments, the insulin, an insulin analog, an insulin agonist and/or an insulin partial agonist is administered at a concentration of 40 μg/ml to 50 μg/ml. In embodiments, the insulin, an insulin analog, an insulin agonist and/or an insulin partial agonist is administered at a concentration of 45 μg/ml to 50 μg/ml.

In embodiments, the insulin, an insulin analog, an insulin agonist and/or an insulin partial agonist is administered at a concentration of 1 μg/ml to 45 μg/ml. In embodiments, the insulin, an insulin analog, an insulin agonist and/or an insulin partial agonist is administered at a concentration of 1 μg/ml to 40 μg/ml. In embodiments, the insulin, an insulin analog, an insulin agonist and/or an insulin partial agonist is administered at a concentration of 1 μg/ml to 35 μg/ml. In embodiments, the insulin, an insulin analog, an insulin agonist and/or an insulin partial agonist is administered at a concentration of 1 μg/ml to 30 μg/ml. In embodiments, the insulin, an insulin analog, an insulin agonist and/or an insulin partial agonist is administered at a concentration of 1 μg/ml to 25 μg/ml. In embodiments, the insulin, an insulin analog, an insulin agonist and/or an insulin partial agonist is administered at a concentration of 1 μg/ml to 20 μg/ml. In embodiments, the insulin, an insulin analog, an insulin agonist and/or an insulin partial agonist is administered at a concentration of 1 μg/ml to 15 μg/ml. In embodiments, the insulin, an insulin analog, an insulin agonist and/or an insulin partial agonist is administered at a concentration of 1 μg/ml to 10 μg/ml. In embodiments, the insulin, an insulin analog, an insulin agonist and/or an insulin partial agonist is administered at a concentration of 1 μg/ml to 9 μg/ml. In embodiments, the insulin, an insulin analog, an insulin agonist and/or an insulin partial agonist is administered at a concentration of 1 μg/ml to 8 μg/ml. In embodiments, the insulin, an insulin analog, an insulin agonist and/or an insulin partial agonist is administered at a concentration of 1 μg/ml to 7 μg/ml. In embodiments, the insulin, an insulin analog, an insulin agonist and/or an insulin partial agonist is administered at a concentration of 1 μg/ml to 6 μg/ml. In embodiments, the insulin, an insulin analog, an insulin agonist and/or an insulin partial agonist is administered at a concentration of 1 μg/ml to 5 μg/ml. In embodiments, the insulin, an insulin analog, an insulin agonist and/or an insulin partial agonist is administered at a concentration of 1 μg/ml to 4 μg/ml. In embodiments, the insulin, an insulin analog, an insulin agonist and/or an insulin partial agonist is administered at a concentration of 1 μg/ml to 3 μg/ml. In embodiments, the insulin, an insulin analog, an insulin agonist and/or an insulin partial agonist is administered at a concentration of 1 μg/ml to 2 μg/ml.

In embodiments, the insulin, an insulin analog, an insulin agonist and/or an insulin partial agonist is administered at a concentration of about 1 μg/ml, about 5 μg/ml, or about 25 μg/ml. In embodiments, the insulin, an insulin analog, an insulin agonist and/or an insulin partial agonist is administered at a concentration of about 1 μg/ml. In embodiments, the insulin, an insulin analog, an insulin agonist and/or an insulin partial agonist is administered at a concentration of about 5 μg/ml. In embodiments, the insulin, an insulin analog, an insulin agonist and/or an insulin partial agonist is administered at a concentration of about 25 μg/ml. In embodiments, the insulin, an insulin analog, an insulin agonist and/or an insulin partial agonist is administered at a concentration of 1 μg/ml, 5 μg/ml, or 25 μg/ml. In embodiments, the insulin, an insulin analog, an insulin agonist and/or an insulin partial agonist is administered at a concentration of 1 μg/ml. In embodiments, the insulin, an insulin analog, an insulin agonist and/or an insulin partial agonist is administered at a concentration of 5 μg/ml. In embodiments, the insulin, an insulin analog, an insulin agonist and/or an insulin partial agonist is administered at a concentration of 25 μg/ml.

The methods provided herein including embodiments thereof may include contacting the T cell with a gene editing reagent, thereby allowing editing of a target gene within the T cell. For example, the gene editing reagent may facilitate knock-out of an endogenous gene (e.g. endogenous TCR) and knock-in of an tumor antigen specific TCR. Thus, in embodiments, the method further includes contacting the T cell with a gene editing reagent. In embodiments, the contacting the T cell with the gene editing reagent includes contacting the T cell with a polynucleotide encoding the gene editing reagent. In embodiments, the T cell is contacted with the polynucleotide in the presence of the gene editing agent or the polynucleotide encoding the gene editing reagent. In embodiments, the T cell is contacted with the polynucleotide in the presence of the gene editing reagent. In embodiments, the T cell is contacted with the polynucleotide in the presence of the polynucleotide encoding the gene editing reagent.

In embodiments, the gene editing reagent includes an RNA-guided nuclease. In embodiments, the RNA-guided nuclease is a CRISPR-Cas system. In embodiments, the CRISPR-Cas system includes Cas1, Cas1B, Cas2, Cas3, Cas4, Cas5, Cas6, Cas7, Cas8, Cas9, Cas10, Cas12, Cas13, nCas9, Cas-CLOVER, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, or Csf4. In embodiments, the CRISPR-Cas system includes Cas1. In embodiments, the CRISPR-Cas system includes Cas1B. In embodiments, the CRISPR-Cas system includes Cas2. In embodiments, the CRISPR-Cas system includes Cas3. In embodiments, the CRISPR-Cas system includes Cas4. In embodiments, the CRISPR-Cas system includes Cas5. In embodiments, the CRISPR-Cas system includes Cas6. In embodiments, the CRISPR-Cas system includes Cas7. In embodiments, the CRISPR-Cas system includes Cas8. In embodiments, the CRISPR-Cas system includes Cas9. In embodiments, the CRISPR-Cas system includes a Cas9 or a Cas9 variant. In embodiments, the CRISPR-Cas system includes a Cas9. In embodiments, the CRISPR-Cas system includes a Cas9 variant. In embodiments, the CRISPR-Cas system includes Cas10. In embodiments, the CRISPR-Cas system includes Cas12. In embodiments, the CRISPR-Cas system includes Cas13. In embodiments, the CRISPR-Cas system includes Csy1. In embodiments, the CRISPR-Cas system includes Csy2. In embodiments, the CRISPR-Cas system includes Csy3. In embodiments, the CRISPR-Cas system includes Cse1. In embodiments, the CRISPR-Cas system includes Cse2. In embodiments, the CRISPR-Cas system includes Csc1. In embodiments, the CRISPR-Cas system includes Csc2. In embodiments, the CRISPR-Cas system includes Csm2. In embodiments, the CRISPR-Cas system includes Csm3. In embodiments, the CRISPR-Cas system includes Csm4. In embodiments, the CRISPR-Cas system includes Csm5. In embodiments, the CRISPR-Cas system includes Csm6. In embodiments, the CRISPR-Cas system includes Cmr1. In embodiments, the CRISPR-Cas system includes Cmr3. In embodiments, the CRISPR-Cas system includes Cmr4. In embodiments, the CRISPR-Cas system includes Cmr5. In embodiments, the CRISPR-Cas system includes Cmr6. In embodiments, the CRISPR-Cas system includes Csb1. In embodiments, the CRISPR-Cas system includes Csb3. In embodiments, the CRISPR-Cas system includes Csx17. In embodiments, the CRISPR-Cas system includes Csx14. In embodiments, the CRISPR-Cas system includes Csx10. In embodiments, the CRISPR-Cas system includes Csx16. In embodiments, the CRISPR-Cas system includes CsaX. In embodiments, the CRISPR-Cas system includes Csx3. In embodiments, the CRISPR-Cas system includes Csx1. In embodiments, the CRISPR-Cas system includes Csx15. In embodiments, the CRISPR-Cas system includes Csf1. In embodiments, the CRISPR-Cas system includes Csf2. In embodiments, the CRISPR-Cas system includes Csf3. In embodiments, the CRISPR-Cas system includes Csf4. In embodiments, the CRISPR-Cas system includes Cas-CLOVER. In embodiments, the CRISPR-Cas system includes nCas9. In embodiments, the gene editing reagent includes a CRISPR-Cas system including a Cas protein and a guide RNA (gRNA).

In embodiments, the gene editing reagent is MAD7, a TALEN, or a ZFN. In embodiments, the gene editing reagent is MAD7. In embodiments, the gene editing reagent is a TALEN. In embodiments, the gene editing reagent is a ZFN. MAD7 is an engineered nuclease of the Class 2 type V-A CRISPR-Cas (Cas12a/Cpf1) family (refseq WP_055225123.1). See, e.g., CRISPR J. April 2020; 3(2): 97-108, which is incorporated herein by reference in its entirety.

In further embodiments, the CRISPR-Cas system includes a Cas enzyme fusion protein. In some embodiments, the fusion protein includes any of the Cas enzymes above. In some embodiments, the fusion protein includes a Cas enzyme and includes an exonuclease. In some embodiments, the fusion protein includes a Cas enzyme and includes a deaminase. In some embodiments, the fusion protein includes a Cas enzyme and includes a DNA repair protein. In some embodiments, the fusion protein includes a Cas enzyme and a chromatin remodeling protein. In some embodiments, the fusion protein includes a Cas enzyme and an NEHJ inhibiting protein. In some embodiments, more than one combination of Cas enzyme fusion proteins are included in the CRISPR-Cas system.

In embodiments, at least 80% of engineered T cells are TCM and/or TSCM. In embodiments, at least 85% of engineered T cells are TCM and/or TSCM. In embodiments, at least 80% of engineered T cells are TCM and/or TSCM. In embodiments, at least 90% of engineered T cells are TCM and/or TSCM. In embodiments, at least 91% of engineered T cells are TCM and/or TSCM. In embodiments, at least 92% of engineered T cells are TCM and/or TSCM. In embodiments, at least 93% of engineered T cells are TCM and/or TSCM. In embodiments, at least 94% of engineered T cells are TCM and/or TSCM. In embodiments, at least 95% of engineered T cells are TCM and/or TSCM. In embodiments, at least 96% of engineered T cells are TCM and/or TSCM. In embodiments, at least 97% of engineered T cells are TCM and/or TSCM. In embodiments, at least 80% of engineered T cells are TCM and/or TSCM. In embodiments, at least 99% of engineered T cells are TCM and/or TSCM.

In embodiments, at least 80% of engineered T cells are TCM and TSCM. In embodiments, at least 85% of engineered T cells are TCM and TSCM. In embodiments, at least 80% of engineered T cells are TCM and TSCM. In embodiments, at least 90% of engineered T cells are TCM and TSCM. In embodiments, at least 91% of engineered T cells are TCM and TSCM. In embodiments, at least 92% of engineered T cells are TCM and TSCM. In embodiments, at least 93% of engineered T cells are TCM and TSCM. In embodiments, at least 94% of engineered T cells are TCM and TSCM. In embodiments, at least 95% of engineered T cells are TCM and TSCM. In embodiments, at least 96% of engineered T cells are TCM and TSCM. In embodiments, at least 97% of engineered T cells are TCM and TSCM. In embodiments, at least 80% of engineered T cells are TCM and TSCM. In embodiments, at least 99% of engineered T cells are TCM and TSCM.

In embodiments, at least 80% of engineered T cells are TCM or TSCM. In embodiments, at least 85% of engineered T cells are TCM or TSCM. In embodiments, at least 80% of engineered T cells are TCM or TSCM. In embodiments, at least 90% of engineered T cells are TCM or TSCM. In embodiments, at least 91% of engineered T cells are TCM or TSCM. In embodiments, at least 92% of engineered T cells are TCM or TSCM. In embodiments, at least 93% of engineered T cells are TCM or TSCM. In embodiments, at least 94% of engineered T cells are TCM or TSCM. In embodiments, at least 95% of engineered T cells are TCM or TSCM. In embodiments, at least 96% of engineered T cells are TCM or TSCM. In embodiments, at least 97% of engineered T cells are TCM or TSCM. In embodiments, at least 80% of engineered T cells are TCM or TSCM. In embodiments, at least 99% of engineered T cells are TCM or TSCM.

In embodiments, at least 80% of engineered T cells are TCM. In embodiments, at least 85% of engineered T cells are TCM. In embodiments, at least 80% of engineered T cells are TCM. In embodiments, at least 90% of engineered T cells are TCM. In embodiments, at least 91% of engineered T cells are TCM. In embodiments, at least 92% of engineered T cells are TCM. In embodiments, at least 93% of engineered T cells are TCM. In embodiments, at least 94% of engineered T cells are TCM. In embodiments, at least 95% of engineered T cells are TCM. In embodiments, at least 96% of engineered T cells are TCM. In embodiments, at least 97% of engineered T cells are TCM. In embodiments, at least 80% of engineered T cells are TCM. In embodiments, at least 99% of engineered T cells are TCM.

In embodiments, at least 80% of engineered T cells are TSCM. In embodiments, at least 85% of engineered T cells are TSCM. In embodiments, at least 80% of engineered T cells are TSCM. In embodiments, at least 90% of engineered T cells are TSCM. In embodiments, at least 91% of engineered T cells are TSCM. In embodiments, at least 92% of engineered T cells are TSCM. In embodiments, at least 93% of engineered T cells are TSCM. In embodiments, at least 94% of engineered T cells are TSCM. In embodiments, at least 95% of engineered T cells are TSCM. In embodiments, at least 96% of engineered T cells are TSCM. In embodiments, at least 97% of engineered T cells are TSCM. In embodiments, at least 80% of engineered T cells are TSCM. In embodiments, at least 99% of engineered T cells are TSCM.

For the methods provided herein, in embodiments, the T cell is cultured in the presence of (contacted with) insulin. In embodiments, the T cell is cultured in the presence of an insulin analog. In embodiments, the insulin analog is an analog as provided herein.

Provided herein, inter alia, are methods for monitoring cell viability for an engineered population of T cells, the method including measuring mitochondrial function and cell metabolism over time. In embodiments, the mitochondrial membrane potential is measured. In embodiments, the mitochondrial membrane potential is measured using a dye-based assay. In embodiments, the dye is JC-1 or JC-10. In embodiments, the dye is JC-1. In embodiments, the dye is JC-10.

Provided herein, inter alia, are methods for increasing the viability of engineered T cells, including contacting a population of T cells in the presence of insulin, insulin analog, insulin agonist, an insulin partial agonist, a gene editing reagent, or a polynucleotide encoding a gene editing reagent thereby forming the population of engineered T cells, wherein the population of engineered T cells has increased cell viability, growth, and/or gene editing efficiencies relative to a population of engineered T cells that are not contacted with insulin, an insulin analog, an insulin agonist, or an insulin partial agonist, wherein the population of engineered T cells are administered to a subject in need thereof. Thus, the methods are contemplated to improve viability of engineered T cells.

“Cell viability” is used in accordance to its ordinary meaning in the arts and refers to the number or proportion of living cells within a population of cells. Cell viability may be assessed by measuring cell proliferation, cell membrane integrity, cell function, or metabolic activity. Cell viability may be measured by contacting the cells with a nucleic acid binding dye that only enters cells that have compromised or damaged cell membrane. Cell viability may be measured by contacting the cells with reagents that react with enzymes in live cells, or reagents that detect cellular glucose metabolism or mitochondrial viability. For example, mitochondrial viability may be determined by measuring the mitochondrial membrane potential using fluorescence detection assays, including assays using a dye-based method of detection, wherein the dye includes JC-1 or JC-10. For example, glucose metabolism may be determined by measuring glucose consumption/uptake, using glucose with heavy isotope carbon (C13), or using a glucose analog, wherein the glucose analog includes 2-NBDG. In embodiments, cell viability may be measured by fluorescence microscopy or flow cytometry. Thus, in an aspect is provided a method of increasing cell viability of a population of engineered T cells, including contacting a population of T cells with insulin and a polynucleotide, thereby forming the population of engineered T cells, wherein the population of engineered T cells has increased cell viability relative to a population of engineered T cells that are not contacted with insulin.

For the methods provided herein, in embodiments, further including contacting the population of T cells with a donor DNA. In embodiments, the polynucleotide encoding the gene editing reagent includes: single-stranded DNA, double-stranded DNA, a linear DNA strand, a plasmid, a nanoplasmid, or a minicircle. In embodiments, the polynucleotide encoding the gene editing reagent includes single-stranded DNA. In embodiments, the polynucleotide encoding the gene editing reagent includes double-stranded DNA. In embodiments, the polynucleotide encoding the gene editing reagent includes a linear DNA strand. In embodiments, the polynucleotide encoding the gene editing reagent includes a plasmid. In embodiments, the polynucleotide encoding the gene editing reagent includes a nanoplasmid. In embodiments, the polynucleotide encoding the gene editing reagent includes a minicircle.

In embodiments, the polynucleotide encoding the gene editing reagent includes a plasmid including a plasmid backbone and a polynucleotide sequence encoding the gene editing reagent. In embodiments, the plasmid further includes the donor DNA. In embodiments, the donor DNA sequence includes a polynucleotide encoding a gene product. In embodiments, the gene product is autologous or allogeneic to the subject. In embodiments, the gene product is autologous to the subject. In embodiments, the gene product is allogeneic to the subject.

In embodiments, the gene product sequence includes a chimeric antigen receptor (CAR), a T cell receptor (TCR), a human leukocyte antigen (HLA), or an alloimmune defense receptor (ADR), or a subunit thereof. In embodiments, the gene product sequence includes a chimeric antigen receptor (CAR). In embodiments, the gene product sequence includes a T cell receptor (TCR). In embodiments, the gene product sequence includes a human leukocyte antigen (HLA). In embodiments, the gene product sequence includes an alloimmune defense receptor (ADR).

In embodiments, the TCR sequence includes an exogenous TCR-beta subunit or fragment thereof and/or an exogenous TCR-alpha subunit or fragment thereof, or a chimeric antigen receptor and/or subunit thereof. In embodiments, the TCR sequence includes an exogenous TCR-beta subunit or fragment thereof and an exogenous TCR-alpha subunit or fragment thereof. In embodiments, the TCR sequence includes an exogenous TCR-beta subunit or fragment thereof or an exogenous TCR-alpha subunit or fragment thereof. In embodiments, the TCR sequence includes an exogenous TCR-beta subunit or fragment thereof. In embodiments, the TCR sequence includes an exogenous TCR-alpha subunit or fragment thereof. In embodiments, the TCR sequence includes a chimeric antigen receptor and subunit thereof. In embodiments, the TCR sequence includes a chimeric antigen receptor or subunit thereof. In embodiments, the TCR sequence is inserted in a TRAC or TRBC locus. In embodiments, the TCR sequence is inserted in a TRAC locus. In embodiments, the TCR sequence is inserted in a TRBC locus.

In embodiments, contacting the population of T cells with the gene editing reagent or polynucleotide encoding the gene reagent includes transfecting the population of T cells with the gene editing reagent or polynucleotide encoding the gene editing reagent. In embodiments, contacting the population of T cells with the gene editing reagent includes transfecting the population of T cells with the gene editing reagent. In embodiments, contacting the population of T cells with the polynucleotide encoding the gene reagent includes transfecting the population of T cells with the polynucleotide encoding the gene editing reagent. In embodiments, the transfecting includes electroporation. In embodiments, the transfecting includes nucleofection. In embodiments, the transfecting includes lipid transfection. In embodiments, the transfecting includes microfluidic transfection.

In embodiments, the population of T cells is cultured in the presence of insulin, an insulin analog, an insulin agonist and/or an insulin partial agonist prior to the contacting step. In embodiments, the population of T cells is cultured in the presence of insulin, an insulin analog, an insulin agonist and an insulin partial agonist prior to the contacting step. In embodiments, the population of T cells is cultured in the presence of insulin, an insulin analog, an insulin agonist or an insulin partial agonist prior to the contacting step. In embodiments, the population of T cells is cultured in the presence of insulin prior to the contacting step. In embodiments, the population of T cells is cultured in the presence of an insulin analog prior to the contacting step. In embodiments, the population of T cells is cultured in the presence of an insulin agonist prior to the contacting step. In embodiments, the population of T cells is cultured in the presence of an insulin partial agonist prior to the contacting step. In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step. In embodiments, including culturing the population of T cells in the presence of insulin, an insulin analog, an insulin agonist and/or an insulin partial agonist for 48 hours prior to the contacting step. In embodiments, including culturing the population of T cells in the presence of insulin, an insulin analog, an insulin agonist and/or an insulin partial agonist for 24 hours prior to the contacting step. In embodiments, including culturing the population of T cells in the presence of insulin, an insulin analog, an insulin agonist and/or an insulin partial agonist for 12 hours prior to the contacting step. In embodiments, including culturing the population of T cells in the presence of insulin, an insulin analog, an insulin agonist and/or an insulin partial agonist for 6 hours prior to the contacting step. In embodiments, including culturing the population of T cells in the presence of insulin, an insulin analog, an insulin agonist and/or an insulin partial agonist for 4 hours prior to the contacting step. In embodiments, including culturing the population of T cells in the presence of insulin, an insulin analog, an insulin agonist and/or an insulin partial agonist for 2 prior to the contacting step. In embodiments, including culturing the population of T cells in the presence of insulin, an insulin analog, an insulin agonist and/or an insulin partial agonist for 1 hour prior to the contacting step. In embodiments, including culturing the population of T cells in the presence of insulin, an insulin analog, an insulin agonist and/or an insulin partial agonist for 30 minutes prior to the contacting step.

In embodiments, the population of T cells is cultured in the presence of insulin, an insulin analog, an insulin agonist and/or an insulin partial agonist after the contacting step. In embodiments, the population of T cells is cultured in the presence of insulin, an insulin analog, an insulin agonist and an insulin partial agonist after the contacting step. In embodiments, the population of T cells is cultured in the presence of insulin, an insulin analog, an insulin agonist or an insulin partial agonist after the contacting step. In embodiments, the population of T cells is cultured in the presence of insulin after the contacting step. In embodiments, the population of T cells is cultured in the presence of an insulin analog after the contacting step. In embodiments, the population of T cells is cultured in the presence of an insulin agonist after the contacting step. In embodiments, the population of T cells is cultured in the presence of an insulin partial agonist after the contacting step. In embodiments, including culturing the population of T cells in the presence of insulin, an insulin analog, an insulin agonist and/or an insulin partial agonist for 48 hours after the contacting step. In embodiments, including culturing the population of T cells in the presence of insulin, an insulin analog, an insulin agonist and/or an insulin partial agonist for 24 hours after the contacting step. In embodiments, including culturing the population of T cells in the presence of insulin, an insulin analog, an insulin agonist and/or an insulin partial agonist for 12 hours after the contacting step. In embodiments, including culturing the population of T cells in the presence of insulin, an insulin analog, an insulin agonist and/or an insulin partial agonist for 6 hours after the contacting step. In embodiments, including culturing the population of T cells in the presence of insulin, an insulin analog, an insulin agonist and/or an insulin partial agonist for 4 hours after the contacting step. In embodiments, including culturing the population of T cells in the presence of insulin, an insulin analog, an insulin agonist and/or an insulin partial agonist for 2 hours after the contacting step. In embodiments, including culturing the population of T cells in the presence of insulin, an insulin analog, an insulin agonist and/or an insulin partial agonist for 1 hour after the contacting step. In embodiments, including culturing the population of T cells in the presence of insulin, an insulin analog, an insulin agonist and/or an insulin partial agonist for 30 minutes after the contacting step.

In embodiments, the population of T cells is cultured in the presence of insulin, an insulin analog, an insulin agonist and/or an insulin partial agonist prior to and after the contacting step. In embodiments, the population of T cells is cultured in the presence of insulin, an insulin analog, an insulin agonist and an insulin partial agonist prior to and after the contacting step. In embodiments, the population of T cells is cultured in the presence of insulin, an insulin analog, an insulin agonist or an insulin partial agonist prior to and after the contacting step. In embodiments, the population of T cells is cultured in the presence of insulin prior to and after the contacting step. In embodiments, the population of T cells is cultured in the presence of an insulin analog prior to and after the contacting step. In embodiments, the population of T cells is cultured in the presence of an insulin agonist prior to and after the contacting step. In embodiments, the population of T cells is cultured in the presence of an insulin partial agonist prior to and after the contacting step.

In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step. In embodiments, including culturing the population of T cells in the presence of insulin, an insulin analog, an insulin agonist and/or an insulin partial agonist for 48 hours prior to the contacting step. In embodiments, including culturing the population of T cells in the presence of insulin, an insulin analog, an insulin agonist and/or an insulin partial agonist for 24 hours prior to the contacting step. In embodiments, including culturing the population of T cells in the presence of insulin, an insulin analog, an insulin agonist and/or an insulin partial agonist for 12 hours prior to the contacting step. In embodiments, including culturing the population of T cells in the presence of insulin, an insulin analog, an insulin agonist and/or an insulin partial agonist for 6 hours prior to the contacting step. In embodiments, including culturing the population of T cells in the presence of insulin, an insulin analog, an insulin agonist and/or an insulin partial agonist for 4 hours prior to the contacting step. In embodiments, including culturing the population of T cells in the presence of insulin, an insulin analog, an insulin agonist and/or an insulin partial agonist for 2 prior to the contacting step. In embodiments, including culturing the population of T cells in the presence of insulin, an insulin analog, an insulin agonist and/or an insulin partial agonist for 1 hour prior to the contacting step. In embodiments, including culturing the population of T cells in the presence of insulin, an insulin analog, an insulin agonist and/or an insulin partial agonist for 30 minutes prior to the contacting step.

In embodiments, including culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes after the contacting step. In embodiments, including culturing the population of T cells in the presence of insulin, an insulin analog, an insulin agonist and/or an insulin partial agonist for 48 hours after the contacting step. In embodiments, including culturing the population of T cells in the presence of insulin, an insulin analog, an insulin agonist and/or an insulin partial agonist for 24 hours after the contacting step. In embodiments, including culturing the population of T cells in the presence of insulin, an insulin analog, an insulin agonist and/or an insulin partial agonist for 12 hours after the contacting step. In embodiments, including culturing the population of T cells in the presence of insulin, an insulin analog, an insulin agonist and/or an insulin partial agonist for 6 hours after the contacting step. In embodiments, including culturing the population of T cells in the presence of insulin, an insulin analog, an insulin agonist and/or an insulin partial agonist for 4 hours after the contacting step. In embodiments, including culturing the population of T cells in the presence of insulin, an insulin analog, an insulin agonist and/or an insulin partial agonist for 2 hours after the contacting step. In embodiments, including culturing the population of T cells in the presence of insulin, an insulin analog, an insulin agonist and/or an insulin partial agonist for 1 hour after the contacting step. In embodiments, including culturing the population of T cells in the presence of insulin, an insulin analog, an insulin agonist and/or an insulin partial agonist for 30 minutes after the contacting step.

In embodiments, the insulin, an insulin analog, an insulin agonist and/or an insulin partial agonist is administered at a concentration of about 1 μg/ml to about 50 μg/ml. In embodiments, the insulin, an insulin analog, an insulin agonist and/or an insulin partial agonist is administered at a concentration of about 2 μg/ml to about 50 μg/ml. In embodiments, the insulin, an insulin analog, an insulin agonist and/or an insulin partial agonist is administered at a concentration of about 3 μg/ml to about 50 μg/ml. In embodiments, the insulin, an insulin analog, an insulin agonist and/or an insulin partial agonist is administered at a concentration of about 4 μg/ml to about 50 μg/ml. In embodiments, the insulin, an insulin analog, an insulin agonist and/or an insulin partial agonist is administered at a concentration of about 5 μg/ml to about 50 μg/ml. In embodiments, the insulin, an insulin analog, an insulin agonist and/or an insulin partial agonist is administered at a concentration of about 6 μg/ml to about 50 μg/ml. In embodiments, the insulin, an insulin analog, an insulin agonist and/or an insulin partial agonist is administered at a concentration of about 7 μg/ml to about 50 μg/ml. In embodiments, the insulin, an insulin analog, an insulin agonist and/or an insulin partial agonist is administered at a concentration of about 8 μg/ml to about 50 μg/ml. In embodiments, the insulin, an insulin analog, an insulin agonist and/or an insulin partial agonist is administered at a concentration of about 9 μg/ml to about 50 μg/ml. In embodiments, the insulin, an insulin analog, an insulin agonist and/or an insulin partial agonist is administered at a concentration of about 10 μg/ml to about 50 μg/ml. In embodiments, the insulin, an insulin analog, an insulin agonist and/or an insulin partial agonist is administered at a concentration of about 15 μg/ml to about 50 μg/ml. In embodiments, the insulin, an insulin analog, an insulin agonist and/or an insulin partial agonist is administered at a concentration of about 20 μg/ml to about 50 μg/ml. In embodiments, the insulin, an insulin analog, an insulin agonist and/or an insulin partial agonist is administered at a concentration of about 25 μg/ml to about 50 μg/ml. In embodiments, the insulin, an insulin analog, an insulin agonist and/or an insulin partial agonist is administered at a concentration of about 30 μg/ml to about 50 μg/ml. In embodiments, the insulin, an insulin analog, an insulin agonist and/or an insulin partial agonist is administered at a concentration of about 35 μg/ml to about 50 μg/ml. In embodiments, the insulin, an insulin analog, an insulin agonist and/or an insulin partial agonist is administered at a concentration of about 40 μg/ml to about 50 μg/ml. In embodiments, the insulin, an insulin analog, an insulin agonist and/or an insulin partial agonist is administered at a concentration of about 45 μg/ml to about 50 μg/ml.

In embodiments, the insulin, an insulin analog, an insulin agonist and/or an insulin partial agonist is administered at a concentration of about 1 μg/ml to about 45 μg/ml. In embodiments, the insulin, an insulin analog, an insulin agonist and/or an insulin partial agonist is administered at a concentration of about 1 μg/ml to about 40 μg/ml. In embodiments, the insulin, an insulin analog, an insulin agonist and/or an insulin partial agonist is administered at a concentration of about 1 μg/ml to about 35 μg/ml. In embodiments, the insulin, an insulin analog, an insulin agonist and/or an insulin partial agonist is administered at a concentration of about 1 μg/ml to about 30 μg/ml. In embodiments, the insulin, an insulin analog, an insulin agonist and/or an insulin partial agonist is administered at a concentration of about 1 μg/ml to about 25 μg/ml. In embodiments, the insulin, an insulin analog, an insulin agonist and/or an insulin partial agonist is administered at a concentration of about 1 μg/ml to about 20 μg/ml. In embodiments, the insulin, an insulin analog, an insulin agonist and/or an insulin partial agonist is administered at a concentration of about 1 μg/ml to about 15 μg/ml. In embodiments, the insulin, an insulin analog, an insulin agonist and/or an insulin partial agonist is administered at a concentration of about 1 μg/ml to about 10 μg/ml. In embodiments, the insulin, an insulin analog, an insulin agonist and/or an insulin partial agonist is administered at a concentration of about 1 μg/ml to about 9 μg/ml. In embodiments, the insulin, an insulin analog, an insulin agonist and/or an insulin partial agonist is administered at a concentration of about 1 μg/ml to about 8 μg/ml. In embodiments, the insulin, an insulin analog, an insulin agonist and/or an insulin partial agonist is administered at a concentration of about 1 μg/ml to about 7 μg/ml. In embodiments, the insulin, an insulin analog, an insulin agonist and/or an insulin partial agonist is administered at a concentration of about 1 μg/ml to about 6 μg/ml. In embodiments, the insulin, an insulin analog, an insulin agonist and/or an insulin partial agonist is administered at a concentration of about 1 μg/ml to about 5 μg/ml. In embodiments, the insulin, an insulin analog, an insulin agonist and/or an insulin partial agonist is administered at a concentration of about 1 μg/ml to about 4 μg/ml. In embodiments, the insulin, an insulin analog, an insulin agonist and/or an insulin partial agonist is administered at a concentration of about 1 μg/ml to about 3 μg/ml. In embodiments, the insulin, an insulin analog, an insulin agonist and/or an insulin partial agonist is administered at a concentration of about 1 μg/ml to about 2 μg/ml.

In embodiments, the insulin, an insulin analog, an insulin agonist and/or an insulin partial agonist is administered at a concentration of 1 μg/ml to 50 μg/ml. In embodiments, the insulin, an insulin analog, an insulin agonist and/or an insulin partial agonist is administered at a concentration of 2 μg/ml to 50 μg/ml. In embodiments, the insulin, an insulin analog, an insulin agonist and/or an insulin partial agonist is administered at a concentration of 3 μg/ml to 50 μg/ml. In embodiments, the insulin, an insulin analog, an insulin agonist and/or an insulin partial agonist is administered at a concentration of 4 μg/ml to 50 μg/ml. In embodiments, the insulin, an insulin analog, an insulin agonist and/or an insulin partial agonist is administered at a concentration of 5 μg/ml to 50 μg/ml. In embodiments, the insulin, an insulin analog, an insulin agonist and/or an insulin partial agonist is administered at a concentration of 6 μg/ml to 50 μg/ml. In embodiments, the insulin, an insulin analog, an insulin agonist and/or an insulin partial agonist is administered at a concentration of 7 μg/ml to 50 μg/ml. In embodiments, the insulin, an insulin analog, an insulin agonist and/or an insulin partial agonist is administered at a concentration of 8 μg/ml to 50 μg/ml. In embodiments, the insulin, an insulin analog, an insulin agonist and/or an insulin partial agonist is administered at a concentration of 9 μg/ml to 50 μg/ml. In embodiments, the insulin, an insulin analog, an insulin agonist and/or an insulin partial agonist is administered at a concentration of 10 μg/ml to 50 μg/ml. In embodiments, the insulin, an insulin analog, an insulin agonist and/or an insulin partial agonist is administered at a concentration of 15 μg/ml to 50 μg/ml. In embodiments, the insulin, an insulin analog, an insulin agonist and/or an insulin partial agonist is administered at a concentration of 20 μg/ml to 50 μg/ml. In embodiments, the insulin, an insulin analog, an insulin agonist and/or an insulin partial agonist is administered at a concentration of 25 μg/ml to 50 μg/ml. In embodiments, the insulin, an insulin analog, an insulin agonist and/or an insulin partial agonist is administered at a concentration of 30 μg/ml to 50 μg/ml. In embodiments, the insulin, an insulin analog, an insulin agonist and/or an insulin partial agonist is administered at a concentration of 35 μg/ml to 50 μg/ml. In embodiments, the insulin, an insulin analog, an insulin agonist and/or an insulin partial agonist is administered at a concentration of 40 μg/ml to 50 μg/ml. In embodiments, the insulin, an insulin analog, an insulin agonist and/or an insulin partial agonist is administered at a concentration of 45 μg/ml to 50 μg/ml.

In embodiments, the insulin, an insulin analog, an insulin agonist and/or an insulin partial agonist is administered at a concentration of 1 μg/ml to 45 μg/ml. In embodiments, the insulin, an insulin analog, an insulin agonist and/or an insulin partial agonist is administered at a concentration of 1 μg/ml to 40 μg/ml. In embodiments, the insulin, an insulin analog, an insulin agonist and/or an insulin partial agonist is administered at a concentration of 1 μg/ml to 35 μg/ml. In embodiments, the insulin, an insulin analog, an insulin agonist and/or an insulin partial agonist is administered at a concentration of 1 μg/ml to 30 μg/ml. In embodiments, the insulin, an insulin analog, an insulin agonist and/or an insulin partial agonist is administered at a concentration of 1 μg/ml to 25 μg/ml. In embodiments, the insulin, an insulin analog, an insulin agonist and/or an insulin partial agonist is administered at a concentration of 1 μg/ml to 20 μg/ml. In embodiments, the insulin, an insulin analog, an insulin agonist and/or an insulin partial agonist is administered at a concentration of 1 μg/ml to 15 μg/ml. In embodiments, the insulin, an insulin analog, an insulin agonist and/or an insulin partial agonist is administered at a concentration of 1 μg/ml to 10 μg/ml. In embodiments, the insulin, an insulin analog, an insulin agonist and/or an insulin partial agonist is administered at a concentration of 1 μg/ml to 9 μg/ml. In embodiments, the insulin, an insulin analog, an insulin agonist and/or an insulin partial agonist is administered at a concentration of 1 μg/ml to 8 μg/ml. In embodiments, the insulin, an insulin analog, an insulin agonist and/or an insulin partial agonist is administered at a concentration of 1 μg/ml to 7 μg/ml. In embodiments, the insulin, an insulin analog, an insulin agonist and/or an insulin partial agonist is administered at a concentration of 1 μg/ml to 6 μg/ml. In embodiments, the insulin, an insulin analog, an insulin agonist and/or an insulin partial agonist is administered at a concentration of 1 μg/ml to 5 μg/ml. In embodiments, the insulin, an insulin analog, an insulin agonist and/or an insulin partial agonist is administered at a concentration of 1 μg/ml to 4 μg/ml. In embodiments, the insulin, an insulin analog, an insulin agonist and/or an insulin partial agonist is administered at a concentration of 1 μg/ml to 3 μg/ml. In embodiments, the insulin, an insulin analog, an insulin agonist and/or an insulin partial agonist is administered at a concentration of 1 μg/ml to 2 μg/ml. Concentration may be any value or subrange within the recited ranges, including endpoints.

In embodiments, the insulin, an insulin analog, an insulin agonist and/or an insulin partial agonist is administered at a concentration of about 1 μg/ml, about 5 μg/ml, or about 25 μg/ml. In embodiments, the insulin, an insulin analog, an insulin agonist and/or an insulin partial agonist is administered at a concentration of about 1 μg/ml. In embodiments, the insulin, an insulin analog, an insulin agonist and/or an insulin partial agonist is administered at a concentration of about 5 μg/ml. In embodiments, the insulin, an insulin analog, an insulin agonist and/or an insulin partial agonist is administered at a concentration of about 25 μg/ml. In embodiments, the insulin, an insulin analog, an insulin agonist and/or an insulin partial agonist is administered at a concentration of 1 μg/ml, 5 μg/ml, or 25 μg/ml. In embodiments, the insulin, an insulin analog, an insulin agonist and/or an insulin partial agonist is administered at a concentration of 1 μg/ml. In embodiments, the insulin, an insulin analog, an insulin agonist and/or an insulin partial agonist is administered at a concentration of 5 μg/ml. In embodiments, the insulin, an insulin analog, an insulin agonist and/or an insulin partial agonist is administered at a concentration of 25 μg/ml.

In embodiments, the population of T cells is transfected with the donor DNA and the gene editing agent or the polynucleotide encoding the gene editing reagent simultaneously. In embodiments, the population of T cells is transfected with the donor DNA and the gene editing agent simultaneously. In embodiments, the population of T cells is transfected with the donor DNA and the polynucleotide encoding the gene editing reagent simultaneously. In embodiments, the gene editing reagent includes an RNA-guided nuclease. In embodiments, the RNA-guided nuclease is a CRISPR-Cas system. In embodiments, the CRISPR-Cas system includes a Cas9 or a Cas9 variant. In embodiments, the CRISPR-Cas system includes a Cas9. In embodiments, the CRISPR-Cas system includes a Cas9 variant. In embodiments, the gene editing reagent includes a CRISPR-Cas system including a Cas protein and a guide RNA (gRNA).

In embodiments, at least 80% of engineered T cells are TCM and/or TSCM. In embodiments, at least 85% of engineered T cells are TCM and/or TSCM. In embodiments, at least 80% of engineered T cells are TCM and/or TSCM. In embodiments, at least 90% of engineered T cells are TCM and/or TSCM. In embodiments, at least 91% of engineered T cells are TCM and/or TSCM. In embodiments, at least 92% of engineered T cells are TCM and/or TSCM. In embodiments, at least 93% of engineered T cells are TCM and/or TSCM. In embodiments, at least 94% of engineered T cells are TCM and/or TSCM. In embodiments, at least 95% of engineered T cells are TCM and/or TSCM. In embodiments, at least 96% of engineered T cells are TCM and/or TSCM. In embodiments, at least 97% of engineered T cells are TCM and/or TSCM. In embodiments, at least 80% of engineered T cells are TCM and/or TSCM. In embodiments, at least 99% of engineered T cells are TCM and/or TSCM.

In embodiments, at least 80% of engineered T cells are TCM and TSCM. In embodiments, at least 85% of engineered T cells are TCM and TSCM. In embodiments, at least 80% of engineered T cells are TCM and TSCM. In embodiments, at least 90% of engineered T cells are TCM and TSCM. In embodiments, at least 91% of engineered T cells are TCM and TSCM. In embodiments, at least 92% of engineered T cells are TCM and TSCM. In embodiments, at least 93% of engineered T cells are TCM and TSCM. In embodiments, at least 94% of engineered T cells are TCM and TSCM. In embodiments, at least 95% of engineered T cells are TCM and TSCM. In embodiments, at least 96% of engineered T cells are TCM and TSCM. In embodiments, at least 97% of engineered T cells are TCM and TSCM. In embodiments, at least 80% of engineered T cells are TCM and TSCM. In embodiments, at least 99% of engineered T cells are TCM and TSCM.

In embodiments, at least 80% of engineered T cells are TCM or TSCM. In embodiments, at least 85% of engineered T cells are TCM or TSCM. In embodiments, at least 80% of engineered T cells are TCM or TSCM. In embodiments, at least 90% of engineered T cells are TCM or TSCM. In embodiments, at least 91% of engineered T cells are TCM or TSCM. In embodiments, at least 92% of engineered T cells are TCM or TSCM. In embodiments, at least 93% of engineered T cells are TCM or TSCM. In embodiments, at least 94% of engineered T cells are TCM or TSCM. In embodiments, at least 95% of engineered T cells are TCM or TSCM. In embodiments, at least 96% of engineered T cells are TCM or TSCM. In embodiments, at least 97% of engineered T cells are TCM or TSCM. In embodiments, at least 80% of engineered T cells are TCM or TSCM. In embodiments, at least 99% of engineered T cells are TCM or TSCM.

In embodiments, at least 80% of engineered T cells are TCM. In embodiments, at least 85% of engineered T cells are TCM. In embodiments, at least 80% of engineered T cells are TCM. In embodiments, at least 90% of engineered T cells are TCM. In embodiments, at least 91% of engineered T cells are TCM. In embodiments, at least 92% of engineered T cells are TCM. In embodiments, at least 93% of engineered T cells are TCM. In embodiments, at least 94% of engineered T cells are TCM. In embodiments, at least 95% of engineered T cells are TCM. In embodiments, at least 96% of engineered T cells are TCM. In embodiments, at least 97% of engineered T cells are TCM. In embodiments, at least 80% of engineered T cells are TCM. In embodiments, at least 99% of engineered T cells are TCM.

In embodiments, at least 80% of engineered T cells are TSCM. In embodiments, at least 85% of engineered T cells are TSCM. In embodiments, at least 80% of engineered T cells are TSCM. In embodiments, at least 90% of engineered T cells are TSCM. In embodiments, at least 91% of engineered T cells are TSCM. In embodiments, at least 92% of engineered T cells are TSCM. In embodiments, at least 93% of engineered T cells are TSCM. In embodiments, at least 94% of engineered T cells are TSCM. In embodiments, at least 95% of engineered T cells are TSCM. In embodiments, at least 96% of engineered T cells are TSCM. In embodiments, at least 97% of engineered T cells are TSCM. In embodiments, at least 80% of engineered T cells are TSCM. In embodiments, at least 99% of engineered T cells are TSCM.

In embodiments, the cell viability and culture performance for the engineered population of T cells is monitored, the method including measuring mitochondrial function and cell metabolism over time. In embodiments, the mitochondrial membrane potential is measured. In embodiments, the mitochondrial membrane potential is measured using a dye-based assay. In embodiments, the dye is JC-1 or JC-10. In embodiments, the dye is JC-1. In embodiments, the dye is JC-10. In embodiments, the cell viability and culture performance for a population of T cells is monitored, the method including measuring a cell metabolism marker over time. In embodiments, the method includes measuring changes in glucose metabolism, Bcl-2 expression, Bcl-XL expression, Bax expression, or Bad expression over time. In embodiments, the In embodiments, the method includes measuring changes in glucose metabolism over time. In embodiments, the method includes measuring changes in Bcl-2 expression over time. In embodiments, the method includes measuring changes in Bcl-XL expression over time. In embodiments, the method includes measuring changes in Bax expression, over time. In embodiments, the method includes measuring changes in Bad expression over time. In embodiments, the engineered population of T cells glucose metabolism is monitored using a glucose analog. In embodiments, the analog is 2-NBDG.

In embodiments, the cell viability of the population of engineered T cells is increased from at least about 0.1 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.2 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.3 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.4 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.5 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.6 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.7 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.8 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 0.9 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 1.0 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 1.5 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 2.0 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 2.5 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 3.0 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 3.5 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 4.0 fold to at least about 5.0 fold relative to a population of engineered T cells that are not cultured in the presence of insulin, an insulin analog, an insulin agonist, or an insulin partial agonist. In embodiments, the cell viability of the population of engineered T cells is increased from at least about 4.5 fold to at least about 5.0 fold relative to a population of engineered T cells that are not cultured in the presence of insulin, an insulin analog, an insulin agonist, or an insulin partial agonist.

In embodiments, the cell viability of the population of engineered T cells is increased from at least about 0.1 fold to at least about 4.5 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 4.0 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 3.5 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 3.0 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 2.5 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 2.0 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 1.5 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 1.0 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.9 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.8 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.7 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.6 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.5 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.4 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.3 fold relative to a population of engineered T cells that are not cultured in the presence of insulin, an insulin analog, an insulin agonist, or an insulin partial agonist. In embodiments, the cell viability of the population of engineered T cells is increased from at least about 0.1 fold to at least about 0.2 fold relative to a population of engineered T cells that are not cultured in the presence of insulin, an insulin analog, an insulin agonist, or an insulin partial agonist.

In embodiments, the cell viability of the population of engineered T cells is increased from at least 0.1 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.2 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.3 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.4 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.5 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.6 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.7 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.8 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 0.9 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 1.0 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 1.5 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 2.0 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 2.5 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 3.0 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 3.5 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 4.0 fold to at least 5.0 fold relative to a population of engineered T cells that are not cultured in the presence of insulin, an insulin analog, an insulin agonist, or an insulin partial agonist. In embodiments, the cell viability of the population of engineered T cells is increased from at least 4.5 fold to at least 5.0 fold relative to a population of engineered T cells that are not cultured in the presence of insulin, an insulin analog, an insulin agonist, or an insulin partial agonist.

In embodiments, the cell viability of the population of engineered T cells is increased from at least 0.1 fold to at least 4.5 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 4.0 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 3.5 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 3.0 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 2.5 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 2.0 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 1.5 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 1.0 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.9 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.8 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.7 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.6 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.5 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.4 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.3 fold relative to a population of engineered T cells that are not cultured in the presence of insulin, an insulin analog, an insulin agonist, or an insulin partial agonist. In embodiments, the cell viability of the population of engineered T cells is increased from at least 0.1 fold to at least 0.2 fold relative to a population of engineered T cells that are not cultured in the presence of insulin, an insulin analog, an insulin agonist, or an insulin partial agonist. Fold increase may be any value or subrange within the recited ranges, including endpoints.

In embodiments, the cell viability is increased about 2.0 fold. In embodiments, the cell viability is increased 2.0 fold. Fold increase may be any value or subrange within the recited ranges, including endpoints.

In embodiments, the cell viability of the population of engineered T cells is from about 30% to about 95%. In embodiments, the cell viability of the population of engineered T cells is from about 35% to about 95%. In embodiments, the cell viability of the population of engineered T cells is from about 40% to about 95%. In embodiments, the cell viability of the population of engineered T cells is from about 45% to about 95%. In embodiments, the cell viability of the population of engineered T cells is from about 50% to about 95%. In embodiments, the cell viability of the population of engineered T cells is from about 55% to about 95%. In embodiments, the cell viability of the population of engineered T cells is from about 60% to about 95%. In embodiments, the cell viability of the population of engineered T cells is from about 65% to about 95%. In embodiments, the cell viability of the population of engineered T cells is from about 70% to about 95%. In embodiments, the cell viability of the population of engineered T cells is from about 75% to about 95%. In embodiments, the cell viability of the population of engineered T cells is from about 80% to about 95%. In embodiments, the cell viability of the population of engineered T cells is from about 85% to about 95%. In embodiments, the cell viability of the population of engineered T cells is from about 90% to about 95%. In embodiments, the cell viability of the population of engineered T cells is from about 91% to about 95%. In embodiments, the cell viability of the population of engineered T cells is from about 92% to about 95%. In embodiments, the cell viability of the population of engineered T cells is from about 93% to about 95%. In embodiments, the cell viability of the population of engineered T cells is from about 94% to about 95%.

In embodiments, the cell viability of the population of engineered T cells is from about 30% to about 94%. In embodiments, the cell viability of the population of engineered T cells is from about 30% to about 93%. In embodiments, the cell viability of the population of engineered T cells is from about 30% to about 92%. In embodiments, the cell viability of the population of engineered T cells is from about 30% to about 91%. In embodiments, the cell viability of the population of engineered T cells is from about 30% to about 90%. In embodiments, the cell viability of the population of engineered T cells is from about 30% to about 85%. In embodiments, the cell viability of the population of engineered T cells is from about 30% to about 80%. In embodiments, the cell viability of the population of engineered T cells is from about 30% to about 75%. In embodiments, the cell viability of the population of engineered T cells is from about 30% to about 70%. In embodiments, the cell viability of the population of engineered T cells is from about 30% to about 65%. In embodiments, the cell viability of the population of engineered T cells is from about 30% to about 60%. In embodiments, the cell viability of the population of engineered T cells is from about 30% to about 55%. In embodiments, the cell viability of the population of engineered T cells is from about 30% to about 50%. In embodiments, the cell viability of the population of engineered T cells is from about 30% to about 45%. In embodiments, the cell viability of the population of engineered T cells is from about 30% to about 40%. In embodiments, the cell viability of the population of engineered T cells is from about 30% to about 35%.

In embodiments, the cell viability of the population of engineered T cells is from 30% to 95%. In embodiments, the cell viability of the population of engineered T cells is from 35% to 95%. In embodiments, the cell viability of the population of engineered T cells is from 40% to 95%. In embodiments, the cell viability of the population of engineered T cells is from 45% to 95%. In embodiments, the cell viability of the population of engineered T cells is from 50% to 95%. In embodiments, the cell viability of the population of engineered T cells is from 55% to 95%. In embodiments, the cell viability of the population of engineered T cells is from 60% to 95%. In embodiments, the cell viability of the population of engineered T cells is from 65% to 95%. In embodiments, the cell viability of the population of engineered T cells is from 70% to 95%. In embodiments, the cell viability of the population of engineered T cells is from 75% to 95%. In embodiments, the cell viability of the population of engineered T cells is from 80% to 95%. In embodiments, the cell viability of the population of engineered T cells is from 85% to 95%. In embodiments, the cell viability of the population of engineered T cells is from 90% to 95%. In embodiments, the cell viability of the population of engineered T cells is from 91% to 95%. In embodiments, the cell viability of the population of engineered T cells is from 92% to 95%. In embodiments, the cell viability of the population of engineered T cells is from 93% to 95%. In embodiments, the cell viability of the population of engineered T cells is from 94% to 95%.

In embodiments, the cell viability of the population of engineered T cells is from 30% to 94%. In embodiments, the cell viability of the population of engineered T cells is from 30% to 93%. In embodiments, the cell viability of the population of engineered T cells is from 30% to 92%. In embodiments, the cell viability of the population of engineered T cells is from 30% to 91%. In embodiments, the cell viability of the population of engineered T cells is from 30% to 90%. In embodiments, the cell viability of the population of engineered T cells is from 30% to 85%. In embodiments, the cell viability of the population of engineered T cells is from 30% to 80%. In embodiments, the cell viability of the population of engineered T cells is from 30% to 75%. In embodiments, the cell viability of the population of engineered T cells is from 30% to 70%. In embodiments, the cell viability of the population of engineered T cells is from 30% to 65%. In embodiments, the cell viability of the population of engineered T cells is from 30% to 60%. In embodiments, the cell viability of the population of engineered T cells is from 30% to 55%. In embodiments, the cell viability of the population of engineered T cells is from 30% to 50%. In embodiments, the cell viability of the population of engineered T cells is from 30% to 45%. In embodiments, the cell viability of the population of engineered T cells is from 30% to 40%. In embodiments, the cell viability of the population of engineered T cells is from 30% to 35%. Percent increase may be any value or subrange within the recited ranges, including endpoints.

Provided herein, inter alia, are methods of increasing gene editing efficiency in a population of engineered T cells, including contacting a population of T cells with insulin, an insulin analog, an insulin agonist, an insulin partial agonist, a gene editing reagent, and a polynucleotide, thereby forming the population of engineered T cells, wherein the population of engineered T cells has increased gene editing efficiency relative to a population of engineered T cells that are not contacted with insulin, insulin analog, insulin agonist, or insulin partial agonist.

In embodiments, the contacting the population of T cells with the polynucleotide includes transfecting the population of T cells with the polynucleotide. In embodiments, the polynucleotide is a donor DNA. In embodiments, the polynucleotide includes: single-stranded DNA, double-stranded DNA, a linear DNA strand, a plasmid, a nanoplasmid, or a minicircle. In embodiments, the polynucleotide includes single-stranded DNA. In embodiments, the polynucleotide includes double-stranded DNA. In embodiments, the polynucleotide e includes a linear DNA strand. In embodiments, the polynucleotide includes a plasmid. In embodiments, the polynucleotide includes a nanoplasmid. In embodiments, the polynucleotide includes a minicircle.

In embodiments, further including contacting the population of T cells with a gene editing reagent. In embodiments, contacting the population of T cells with the gene editing reagent includes transfecting the population of T cells with the gene editing reagent or a polynucleotide encoding the gene editing reagent. In embodiments, contacting the population of T cells with the gene editing reagent includes transfecting the population of T cells with the gene editing reagent. In embodiments, contacting the population of T cells with the gene editing reagent includes transfecting the population of T cells with a polynucleotide encoding the gene editing reagent. In embodiments, the population of T cells is transfected with the polynucleotide and the gene editing agent or the polynucleotide encoding the gene editing reagent simultaneously. In embodiments, the population of T cells is transfected with the polynucleotide and the gene editing agent simultaneously. In embodiments, the population of T cells is transfected with the polynucleotide and the polynucleotide encoding the gene editing reagent simultaneously.

In embodiments, the insulin, an insulin analog, an insulin agonist and/or an insulin partial agonist is administered at a concentration of about 1 μg/ml to about 50 μg/ml. In embodiments, the insulin, an insulin analog, an insulin agonist and/or an insulin partial agonist is administered at a concentration of about 2 μg/ml to about 50 μg/ml. In embodiments, the insulin, an insulin analog, an insulin agonist and/or an insulin partial agonist is administered at a concentration of about 3 μg/ml to about 50 μg/ml. In embodiments, the insulin, an insulin analog, an insulin agonist and/or an insulin partial agonist is administered at a concentration of about 4 μg/ml to about 50 μg/ml. In embodiments, the insulin, an insulin analog, an insulin agonist and/or an insulin partial agonist is administered at a concentration of about 5 μg/ml to about 50 μg/ml. In embodiments, the insulin, an insulin analog, an insulin agonist and/or an insulin partial agonist is administered at a concentration of about 6 μg/ml to about 50 μg/ml. In embodiments, the insulin, an insulin analog, an insulin agonist and/or an insulin partial agonist is administered at a concentration of about 7 μg/ml to about 50 μg/ml. In embodiments, the insulin, an insulin analog, an insulin agonist and/or an insulin partial agonist is administered at a concentration of about 8 μg/ml to about 50 μg/ml. In embodiments, the insulin, an insulin analog, an insulin agonist and/or an insulin partial agonist is administered at a concentration of about 9 μg/ml to about 50 μg/ml. In embodiments, the insulin, an insulin analog, an insulin agonist and/or an insulin partial agonist is administered at a concentration of about 10 μg/ml to about 50 μg/ml. In embodiments, the insulin, an insulin analog, an insulin agonist and/or an insulin partial agonist is administered at a concentration of about 15 μg/ml to about 50 μg/ml. In embodiments, the insulin, an insulin analog, an insulin agonist and/or an insulin partial agonist is administered at a concentration of about 20 μg/ml to about 50 μg/ml. In embodiments, the insulin, an insulin analog, an insulin agonist and/or an insulin partial agonist is administered at a concentration of about 25 μg/ml to about 50 μg/ml. In embodiments, the insulin, an insulin analog, an insulin agonist and/or an insulin partial agonist is administered at a concentration of about 30 μg/ml to about 50 μg/ml. In embodiments, the insulin, an insulin analog, an insulin agonist and/or an insulin partial agonist is administered at a concentration of about 35 μg/ml to about 50 μg/ml. In embodiments, the insulin, an insulin analog, an insulin agonist and/or an insulin partial agonist is administered at a concentration of about 40 μg/ml to about 50 μg/ml. In embodiments, the insulin, an insulin analog, an insulin agonist and/or an insulin partial agonist is administered at a concentration of about 45 μg/ml to about 50 μg/ml.

In embodiments, the insulin, an insulin analog, an insulin agonist and/or an insulin partial agonist is administered at a concentration of about 1 μg/ml to about 45 μg/ml. In embodiments, the insulin, an insulin analog, an insulin agonist and/or an insulin partial agonist is administered at a concentration of about 1 μg/ml to about 40 μg/ml. In embodiments, the insulin, an insulin analog, an insulin agonist and/or an insulin partial agonist is administered at a concentration of about 1 μg/ml to about 35 μg/ml. In embodiments, the insulin, an insulin analog, an insulin agonist and/or an insulin partial agonist is administered at a concentration of about 1 μg/ml to about 30 μg/ml. In embodiments, the insulin, an insulin analog, an insulin agonist and/or an insulin partial agonist is administered at a concentration of about 1 μg/ml to about 25 μg/ml. In embodiments, the insulin, an insulin analog, an insulin agonist and/or an insulin partial agonist is administered at a concentration of about 1 μg/ml to about 20 μg/ml. In embodiments, the insulin, an insulin analog, an insulin agonist and/or an insulin partial agonist is administered at a concentration of about 1 μg/ml to about 15 μg/ml. In embodiments, the insulin, an insulin analog, an insulin agonist and/or an insulin partial agonist is administered at a concentration of about 1 μg/ml to about 10 μg/ml. In embodiments, the insulin, an insulin analog, an insulin agonist and/or an insulin partial agonist is administered at a concentration of about 1 μg/ml to about 9 μg/ml. In embodiments, the insulin, an insulin analog, an insulin agonist and/or an insulin partial agonist is administered at a concentration of about 1 μg/ml to about 8 μg/ml. In embodiments, the insulin, an insulin analog, an insulin agonist and/or an insulin partial agonist is administered at a concentration of about 1 μg/ml to about 7 μg/ml. In embodiments, the insulin, an insulin analog, an insulin agonist and/or an insulin partial agonist is administered at a concentration of about 1 μg/ml to about 6 μg/ml. In embodiments, the insulin, an insulin analog, an insulin agonist and/or an insulin partial agonist is administered at a concentration of about 1 μg/ml to about 5 μg/ml. In embodiments, the insulin, an insulin analog, an insulin agonist and/or an insulin partial agonist is administered at a concentration of about 1 μg/ml to about 4 μg/ml. In embodiments, the insulin, an insulin analog, an insulin agonist and/or an insulin partial agonist is administered at a concentration of about 1 μg/ml to about 3 μg/ml. In embodiments, the insulin, an insulin analog, an insulin agonist and/or an insulin partial agonist is administered at a concentration of about 1 μg/ml to about 2 μg/ml.

In embodiments, the insulin, an insulin analog, an insulin agonist and/or an insulin partial agonist is administered at a concentration of 1 μg/ml to 50 μg/ml. In embodiments, the insulin, an insulin analog, an insulin agonist and/or an insulin partial agonist is administered at a concentration of 2 μg/ml to 50 μg/ml. In embodiments, the insulin, an insulin analog, an insulin agonist and/or an insulin partial agonist is administered at a concentration of 3 μg/ml to 50 μg/ml. In embodiments, the insulin, an insulin analog, an insulin agonist and/or an insulin partial agonist is administered at a concentration of 4 μg/ml to 50 μg/ml. In embodiments, the insulin, an insulin analog, an insulin agonist and/or an insulin partial agonist is administered at a concentration of 5 μg/ml to 50 μg/ml. In embodiments, the insulin, an insulin analog, an insulin agonist and/or an insulin partial agonist is administered at a concentration of 6 μg/ml to 50 μg/ml. In embodiments, the insulin, an insulin analog, an insulin agonist and/or an insulin partial agonist is administered at a concentration of 7 μg/ml to 50 μg/ml. In embodiments, the insulin, an insulin analog, an insulin agonist and/or an insulin partial agonist is administered at a concentration of 8 μg/ml to 50 μg/ml. In embodiments, the insulin, an insulin analog, an insulin agonist and/or an insulin partial agonist is administered at a concentration of 9 μg/ml to 50 μg/ml. In embodiments, the insulin, an insulin analog, an insulin agonist and/or an insulin partial agonist is administered at a concentration of 10 μg/ml to 50 μg/ml. In embodiments, the insulin, an insulin analog, an insulin agonist and/or an insulin partial agonist is administered at a concentration of 15 μg/ml to 50 μg/ml. In embodiments, the insulin, an insulin analog, an insulin agonist and/or an insulin partial agonist is administered at a concentration of 20 μg/ml to 50 μg/ml. In embodiments, the insulin, an insulin analog, an insulin agonist and/or an insulin partial agonist is administered at a concentration of 25 μg/ml to 50 μg/ml. In embodiments, the insulin, an insulin analog, an insulin agonist and/or an insulin partial agonist is administered at a concentration of 30 μg/ml to 50 μg/ml. In embodiments, the insulin, an insulin analog, an insulin agonist and/or an insulin partial agonist is administered at a concentration of 35 μg/ml to 50 μg/ml. In embodiments, the insulin, an insulin analog, an insulin agonist and/or an insulin partial agonist is administered at a concentration of 40 μg/ml to 50 μg/ml. In embodiments, the insulin, an insulin analog, an insulin agonist and/or an insulin partial agonist is administered at a concentration of 45 μg/ml to 50 μg/ml.

In embodiments, the insulin, an insulin analog, an insulin agonist and/or an insulin partial agonist is administered at a concentration of 1 μg/ml to 45 μg/ml. In embodiments, the insulin, an insulin analog, an insulin agonist and/or an insulin partial agonist is administered at a concentration of 1 μg/ml to 40 μg/ml. In embodiments, the insulin, an insulin analog, an insulin agonist and/or an insulin partial agonist is administered at a concentration of 1 μg/ml to 35 μg/ml. In embodiments, the insulin, an insulin analog, an insulin agonist and/or an insulin partial agonist is administered at a concentration of 1 μg/ml to 30 μg/ml. In embodiments, the insulin, an insulin analog, an insulin agonist and/or an insulin partial agonist is administered at a concentration of 1 μg/ml to 25 μg/ml. In embodiments, the insulin, an insulin analog, an insulin agonist and/or an insulin partial agonist is administered at a concentration of 1 μg/ml to 20 μg/ml. In embodiments, the insulin, an insulin analog, an insulin agonist and/or an insulin partial agonist is administered at a concentration of 1 μg/ml to 15 μg/ml. In embodiments, the insulin, an insulin analog, an insulin agonist and/or an insulin partial agonist is administered at a concentration of 1 μg/ml to 10 μg/ml. In embodiments, the insulin, an insulin analog, an insulin agonist and/or an insulin partial agonist is administered at a concentration of 1 μg/ml to 9 μg/ml. In embodiments, the insulin, an insulin analog, an insulin agonist and/or an insulin partial agonist is administered at a concentration of 1 μg/ml to 8 μg/ml. In embodiments, the insulin, an insulin analog, an insulin agonist and/or an insulin partial agonist is administered at a concentration of 1 μg/ml to 7 μg/ml. In embodiments, the insulin, an insulin analog, an insulin agonist and/or an insulin partial agonist is administered at a concentration of 1 μg/ml to 6 μg/ml. In embodiments, the insulin, an insulin analog, an insulin agonist and/or an insulin partial agonist is administered at a concentration of 1 μg/ml to 5 μg/ml. In embodiments, the insulin, an insulin analog, an insulin agonist and/or an insulin partial agonist is administered at a concentration of 1 μg/ml to 4 μg/ml. In embodiments, the insulin, an insulin analog, an insulin agonist and/or an insulin partial agonist is administered at a concentration of 1 μg/ml to 3 μg/ml. In embodiments, the insulin, an insulin analog, an insulin agonist and/or an insulin partial agonist is administered at a concentration of 1 μg/ml to 2 μg/ml. Concentration may be any value or subrange within the recited ranges, including endpoints.

In embodiments, the population of T cells is contacted simultaneously with the polynucleotide and insulin, an insulin analog, an insulin agonist, and/or an insulin partial agonist. In embodiments, the population of T cells is contacted sequentially with the polynucleotide and insulin, an insulin analog, an insulin agonist, and/or an insulin partial agonist. In embodiments, the population of T cells is contacted with insulin inhibitors prior to the polynucleotide.

In embodiments, the gene editing efficiency of the population of engineered T cells is increased from at least about 0.1 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.2 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.3 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.4 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.5 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.6 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.7 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.8 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.9 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 1.0 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 1.5 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 2.0 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 2.5 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 3.0 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 3.5 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 4.0 fold to at least about 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 4.5 fold to at least about 5.0 fold relative to a population of engineered T cells that are not cultured in the presence of insulin, an insulin analog, an insulin agonist, or an insulin partial agonist.

In embodiments, the gene editing efficiency of the population of engineered T cells is increased from at least about 0.1 fold to at least about 4.5 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 4.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 3.5 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 3.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 2.5 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 2.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 1.5 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 1.0 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.9 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.8 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.7 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.6 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.5 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.4 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.3 fold relative to a population of engineered T cells that are not cultured in the presence of insulin, an insulin analog, an insulin agonist, or an insulin partial agonist. In embodiments, the gene editing efficiency of the population of engineered T cells is increased from at least about 0.1 fold to at least about 0.2 fold relative to a population of engineered T cells that are not cultured in the presence of insulin, an insulin analog, an insulin agonist, or an insulin partial agonist.

In embodiments, the gene editing efficiency of the population of engineered T cells is increased from at least 0.1 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.2 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.3 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.4 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.5 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.6 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.7 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.8 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.9 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 1.0 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 1.5 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 2.0 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 2.5 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 3.0 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 3.5 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 4.0 fold to at least 5.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 4.5 fold to at least 5.0 fold relative to a population of engineered T cells that are not cultured in the presence of insulin, an insulin analog, an insulin agonist, or an insulin partial agonist.

In embodiments, the gene editing efficiency of the population of engineered T cells is increased from at least 0.1 fold to at least 4.5 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 4.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 3.5 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 3.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 2.5 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 2.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 1.5 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 1.0 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.9 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.8 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.7 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.6 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.5 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.4 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.3 fold relative to a population of engineered T cells that are not cultured in the presence of insulin, an insulin analog, an insulin agonist, or an insulin partial agonist. In embodiments, the gene editing efficiency of the population of engineered T cells is increased from at least 0.1 fold to at least 0.2 fold relative to a population of engineered T cells that are not cultured in the presence of insulin, an insulin analog, an insulin agonist, or an insulin partial agonist. Fold increase may be any value or subrange within the recited ranges, including endpoints.

In embodiments, the gene editing efficiency of the population of engineered T cells is increased from about 2 fold to about 3 fold. In embodiments, the gene editing efficiency of the population of engineered T cells is increased from 2 fold to 3 fold. Fold increase may be any value or subrange within the recited ranges, including endpoints.

In embodiments, the gene editing efficiency of the population of engineered T cells is from about 1% to about 99%. In embodiments, the gene editing efficiency of the population of engineered T cells is from about 5% to about 99%. In embodiments, the gene editing efficiency of the population of engineered T cells is from about 10% to about 99%. In embodiments, the gene editing efficiency of the population of engineered T cells is from about 20% to about 99%. In embodiments, the gene editing efficiency of the population of engineered T cells is from about 30% to about 99%. In embodiments, the gene editing efficiency of the population of engineered T cells is from about 40% to about 99%. In embodiments, the gene editing efficiency of the population of engineered T cells is from about 50% to about 99%. In embodiments, the gene editing efficiency of the population of engineered T cells is from about 60% to about 99%. In embodiments, the gene editing efficiency of the population of engineered T cells is from about 70% to about 99%. In embodiments, the gene editing efficiency of the population of engineered T cells is from about 80% to about 99%. In embodiments, the gene editing efficiency of the population of engineered T cells is from about 90% to about 99%. In embodiments, the gene editing efficiency of the population of engineered T cells is from about 91% to about 99%. In embodiments, the gene editing efficiency of the population of engineered T cells is from about 92% to about 99%. In embodiments, the gene editing efficiency of the population of engineered T cells is from about 93 to about 99%. In embodiments, the gene editing efficiency of the population of engineered T cells is from about 94% to about 99%. In embodiments, the gene editing efficiency of the population of engineered T cells is from about 95% to about 99%. In embodiments, the gene editing efficiency of the population of engineered T cells is from about 96% to about 99%. In embodiments, the gene editing efficiency of the population of engineered T cells is from about 97% to about 99%. In embodiments, the gene editing efficiency of the population of engineered T cells is from about 98% to about 99%.

In embodiments, the gene editing efficiency of the population of engineered T cells is from about 1% to about 98%. In embodiments, the gene editing efficiency of the population of engineered T cells is from about 1% to about 97%. In embodiments, the gene editing efficiency of the population of engineered T cells is from about 1% to about 96%. In embodiments, the gene editing efficiency of the population of engineered T cells is from about 1% to about 95%. In embodiments, the gene editing efficiency of the population of engineered T cells is from about 1% to about 94%. In embodiments, the gene editing efficiency of the population of engineered T cells is from about 1% to about 93%. In embodiments, the gene editing efficiency of the population of engineered T cells is from about 1% to about 92%. In embodiments, the gene editing efficiency of the population of engineered T cells is from about 1% to about 91%. In embodiments, the gene editing efficiency of the population of engineered T cells is from about 1% to about 90%. In embodiments, the gene editing efficiency of the population of engineered T cells is from about 1% to about 80%. In embodiments, the gene editing efficiency of the population of engineered T cells is from about 1% to about 70%. In embodiments, the gene editing efficiency of the population of engineered T cells is from about 1% to about 60%. In embodiments, the gene editing efficiency of the population of engineered T cells is from about 1% to about 50%. In embodiments, the gene editing efficiency of the population of engineered T cells is from about 1% to about 40%. In embodiments, the gene editing efficiency of the population of engineered T cells is from about 1% to about 30%. In embodiments, the gene editing efficiency of the population of engineered T cells is from about 1% to about 20%. In embodiments, the gene editing efficiency of the population of engineered T cells is from about 1% to about 10%. In embodiments, the gene editing efficiency of the population of engineered T cells is from about 1% to about 5%.

In embodiments, the gene editing efficiency of the population of engineered T cells is from 1% to 99%. In embodiments, the gene editing efficiency of the population of engineered T cells is from 5% to 99%. In embodiments, the gene editing efficiency of the population of engineered T cells is from 10% to 99%. In embodiments, the gene editing efficiency of the population of engineered T cells is from 20% to 99%. In embodiments, the gene editing efficiency of the population of engineered T cells is from 30% to 99%. In embodiments, the gene editing efficiency of the population of engineered T cells is from 40% to 99%. In embodiments, the gene editing efficiency of the population of engineered T cells is from 50% to 99%. In embodiments, the gene editing efficiency of the population of engineered T cells is from 60% to 99%. In embodiments, the gene editing efficiency of the population of engineered T cells is from 70% to 99%. In embodiments, the gene editing efficiency of the population of engineered T cells is from 80% to 99%. In embodiments, the gene editing efficiency of the population of engineered T cells is from 90% to 99%. In embodiments, the gene editing efficiency of the population of engineered T cells is from 91% to 99%. In embodiments, the gene editing efficiency of the population of engineered T cells is from 92% to 99%. In embodiments, the gene editing efficiency of the population of engineered T cells is from 93 to 99%. In embodiments, the gene editing efficiency of the population of engineered T cells is from 94% to 99%. In embodiments, the gene editing efficiency of the population of engineered T cells is from 95% to 99%. In embodiments, the gene editing efficiency of the population of engineered T cells is from 96% to 99%. In embodiments, the gene editing efficiency of the population of engineered T cells is from 97% to 99%. In embodiments, the gene editing efficiency of the population of engineered T cells is from 98% to 99%.

In embodiments, the gene editing efficiency of the population of engineered T cells is from 1% to 98%. In embodiments, the gene editing efficiency of the population of engineered T cells is from 1% to 97%. In embodiments, the gene editing efficiency of the population of engineered T cells is from 1% to 96%. In embodiments, the gene editing efficiency of the population of engineered T cells is from 1% to 95%. In embodiments, the gene editing efficiency of the population of engineered T cells is from 1% to 94%. In embodiments, the gene editing efficiency of the population of engineered T cells is from 1% to 93%. In embodiments, the gene editing efficiency of the population of engineered T cells is from 1% to 92%. In embodiments, the gene editing efficiency of the population of engineered T cells is from 1% to 91%. In embodiments, the gene editing efficiency of the population of engineered T cells is from 1% to 90%. In embodiments, the gene editing efficiency of the population of engineered T cells is from 1% to 80%. In embodiments, the gene editing efficiency of the population of engineered T cells is from 1% to 70%. In embodiments, the gene editing efficiency of the population of engineered T cells is from 1% to 60%. In embodiments, the gene editing efficiency of the population of engineered T cells is from 1% to 50%. In embodiments, the gene editing efficiency of the population of engineered T cells is from 1% to 40%. In embodiments, the gene editing efficiency of the population of engineered T cells is from 1% to 30%. In embodiments, the gene editing efficiency of the population of engineered T cells is from 1% to 20%. In embodiments, the gene editing efficiency of the population of engineered T cells is from 1% to 10%. In embodiments, the gene editing efficiency of the population of engineered T cells is from 1% to 5%. Percent increase may be any value or subrange within the recited ranges, including endpoints.

In embodiments, knock-out efficiency of the population of engineered T cells is from about 70% to about 99%. In embodiments, knock-out efficiency of the population of engineered T cells is from about 75% to about 99%. In embodiments, knock-out efficiency of the population of engineered T cells is from about 80% to about 99%. In embodiments, knock-out efficiency of the population of engineered T cells is from about 85% to about 99%. In embodiments, knock-out efficiency of the population of engineered T cells is from about 90% to about 99%. In embodiments, knock-out efficiency of the population of engineered T cells is from about 91% to about 99%. In embodiments, knock-out efficiency of the population of engineered T cells is from about 92% to about 99%. In embodiments, knock-out efficiency of the population of engineered T cells is from about 93% to about 99%. In embodiments, knock-out efficiency of the population of engineered T cells is from about 94% to about 99%. In embodiments, knock-out efficiency of the population of engineered T cells is from about 95% to about 99%. In embodiments, knock-out efficiency of the population of engineered T cells is from about 96% to about 99%. In embodiments, knock-out efficiency of the population of engineered T cells is from about 97% to about 99%. In embodiments, knock-out efficiency of the population of engineered T cells is from about 98% to about 99%.

In embodiments, knock-out efficiency of the population of engineered T cells is from about 70% to about 98%. In embodiments, knock-out efficiency of the population of engineered T cells is from about 70% to about 97%. In embodiments, knock-out efficiency of the population of engineered T cells is from about 70% to about 96%. In embodiments, knock-out efficiency of the population of engineered T cells is from about 70% to about 95%. In embodiments, knock-out efficiency of the population of engineered T cells is from about 70% to about 94%. In embodiments, knock-out efficiency of the population of engineered T cells is from about 70% to about 93%. In embodiments, knock-out efficiency of the population of engineered T cells is from about 70% to about 92%. In embodiments, knock-out efficiency of the population of engineered T cells is from about 70% to about 91%. In embodiments, knock-out efficiency of the population of engineered T cells is from about 70% to about 90%. In embodiments, knock-out efficiency of the population of engineered T cells is from about 70% to about 85%. In embodiments, knock-out efficiency of the population of engineered T cells is from about 70% to about 80%. In embodiments, knock-out efficiency of the population of engineered T cells is from about 70% to about 75%.

In embodiments, knock-out efficiency of the population of engineered T cells is from 70% to 99%. In embodiments, knock-out efficiency of the population of engineered T cells is from 75% to 99%. In embodiments, knock-out efficiency of the population of engineered T cells is from 80% to 99%. In embodiments, knock-out efficiency of the population of engineered T cells is from 85% to 99%. In embodiments, knock-out efficiency of the population of engineered T cells is from 90% to 99%. In embodiments, knock-out efficiency of the population of engineered T cells is from 91% to 99%. In embodiments, knock-out efficiency of the population of engineered T cells is from 92% to 99%. In embodiments, knock-out efficiency of the population of engineered T cells is from 93% to 99%. In embodiments, knock-out efficiency of the population of engineered T cells is from 94% to 99%. In embodiments, knock-out efficiency of the population of engineered T cells is from 95% to 99%. In embodiments, knock-out efficiency of the population of engineered T cells is from 96% to 99%. In embodiments, knock-out efficiency of the population of engineered T cells is from 97% to 99%. In embodiments, knock-out efficiency of the population of engineered T cells is from 98% to 99%.

In embodiments, knock-out efficiency of the population of engineered T cells is from 70% to 98%. In embodiments, knock-out efficiency of the population of engineered T cells is from 70% to 97%. In embodiments, knock-out efficiency of the population of engineered T cells is from 70% to 96%. In embodiments, knock-out efficiency of the population of engineered T cells is from 70% to 95%. In embodiments, knock-out efficiency of the population of engineered T cells is from 70% to 94%. In embodiments, knock-out efficiency of the population of engineered T cells is from 70% to 93%. In embodiments, knock-out efficiency of the population of engineered T cells is from 70% to 92%. In embodiments, knock-out efficiency of the population of engineered T cells is from 70% to 91%. In embodiments, knock-out efficiency of the population of engineered T cells is from 70% to 90%. In embodiments, knock-out efficiency of the population of engineered T cells is from 70% to 85%. In embodiments, knock-out efficiency of the population of engineered T cells is from 70% to 80%. In embodiments, knock-out efficiency of the population of engineered T cells is from 70% to 75%. Percent knock-out efficiency may be any value or subrange within the recited ranges, including endpoints. For the methods provided herein, in embodiments, the knock-out efficiency is about 90%. In embodiments, the knock-out efficiency is 90%.

In embodiments, knock-in efficiency of the population of engineered T cells is from about 1% to about 99%. In embodiments, knock-in efficiency of the population of engineered T cells is from about 5% to about 99%. In embodiments, knock-in efficiency of the population of engineered T cells is from about 10% to about 99%. In embodiments, knock-in efficiency of the population of engineered T cells is from about 20% to about 99%. In embodiments, knock-in efficiency of the population of engineered T cells is from about 30% to about 99%. In embodiments, knock-in efficiency of the population of engineered T cells is from about 40% to about 99%. In embodiments, knock-in efficiency of the population of engineered T cells is from about 50% to about 99%. In embodiments, knock-in efficiency of the population of engineered T cells is from about 60% to about 99%. In embodiments, knock-in efficiency of the population of engineered T cells is from about 70% to about 99%. In embodiments, knock-in efficiency of the population of engineered T cells is from about 80% to about 99%. In embodiments, knock-in efficiency of the population of engineered T cells is from about 90% to about 99%. In embodiments, knock-in efficiency of the population of engineered T cells is from about 91% to about 99%. In embodiments, knock-in efficiency of the population of engineered T cells is from about 92% to about 99%. In embodiments, knock-in efficiency of the population of engineered T cells is from about 93% to about 99%. In embodiments, knock-in efficiency of the population of engineered T cells is from about 94% to about 99%. In embodiments, knock-in efficiency of the population of engineered T cells is from about 95% to about 99%. In embodiments, knock-in efficiency of the population of engineered T cells is from about 96% to about 99%. In embodiments, knock-in efficiency of the population of engineered T cells is from about 97% to about 99%. In embodiments, knock-in efficiency of the population of engineered T cells is from about 98% to about 99%.

In embodiments, knock-in efficiency of the population of engineered T cells is from about 1% to about 98%. In embodiments, knock-in efficiency of the population of engineered T cells is from about 1% to about 97%. In embodiments, knock-in efficiency of the population of engineered T cells is from about 1% to about 96%. In embodiments, knock-in efficiency of the population of engineered T cells is from about 1% to about 95%. In embodiments, knock-in efficiency of the population of engineered T cells is from about 1% to about 94%. In embodiments, knock-in efficiency of the population of engineered T cells is from about 1% to about 93%. In embodiments, knock-in efficiency of the population of engineered T cells is from about 1% to about 92%. In embodiments, knock-in efficiency of the population of engineered T cells is from about 1% to about 91%. In embodiments, knock-in efficiency of the population of engineered T cells is from about 1% to about 90%. In embodiments, knock-in efficiency of the population of engineered T cells is from about 1% to about 80%. In embodiments, knock-in efficiency of the population of engineered T cells is from about 1% to about 70%. In embodiments, knock-in efficiency of the population of engineered T cells is from about 1% to about 60%. In embodiments, knock-in efficiency of the population of engineered T cells is from about 1% to about 50%. In embodiments, knock-in efficiency of the population of engineered T cells is from about 1% to about 40%. In embodiments, knock-in efficiency of the population of engineered T cells is from about 1% to about 30%. In embodiments, knock-in efficiency of the population of engineered T cells is from about 1% to about 20%. In embodiments, knock-in efficiency of the population of engineered T cells is from about 1% to about 10%. In embodiments, knock-in efficiency of the population of engineered T cells is from about 1% to about 5%

In embodiments, knock-in efficiency of the population of engineered T cells is from 1% to 99%. In embodiments, knock-in efficiency of the population of engineered T cells is from 5% to 99%. In embodiments, knock-in efficiency of the population of engineered T cells is from 10% to 99%. In embodiments, knock-in efficiency of the population of engineered T cells is from 20% to 99%. In embodiments, knock-in efficiency of the population of engineered T cells is from 30% to 99%. In embodiments, knock-in efficiency of the population of engineered T cells is from 40% to 99%. In embodiments, knock-in efficiency of the population of engineered T cells is from 50% to 99%. In embodiments, knock-in efficiency of the population of engineered T cells is from 60% to 99%. In embodiments, knock-in efficiency of the population of engineered T cells is from 70% to 99%. In embodiments, knock-in efficiency of the population of engineered T cells is from 80% to 99%. In embodiments, knock-in efficiency of the population of engineered T cells is from 90% to 99%. In embodiments, knock-in efficiency of the population of engineered T cells is from 91% to 99%. In embodiments, knock-in efficiency of the population of engineered T cells is from 92% to 99%. In embodiments, knock-in efficiency of the population of engineered T cells is from 93% to 99%. In embodiments, knock-in efficiency of the population of engineered T cells is from 94% to 99%. In embodiments, knock-in efficiency of the population of engineered T cells is from 95% to 99%. In embodiments, knock-in efficiency of the population of engineered T cells is from 96% to 99%. In embodiments, knock-in efficiency of the population of engineered T cells is from 97% to 99%. In embodiments, knock-in efficiency of the population of engineered T cells is from 98% to 99%.

In embodiments, knock-in efficiency of the population of engineered T cells is from 1% to 98%. In embodiments, knock-in efficiency of the population of engineered T cells is from 1% to 97%. In embodiments, knock-in efficiency of the population of engineered T cells is from 1% to 96%. In embodiments, knock-in efficiency of the population of engineered T cells is from 1% to 95%. In embodiments, knock-in efficiency of the population of engineered T cells is from 1% to 94%. In embodiments, knock-in efficiency of the population of engineered T cells is from 1% to 93%. In embodiments, knock-in efficiency of the population of engineered T cells is from 1% to 92%. In embodiments, knock-in efficiency of the population of engineered T cells is from 1% to 91%. In embodiments, knock-in efficiency of the population of engineered T cells is from 1% to 90%. In embodiments, knock-in efficiency of the population of engineered T cells is from 1% to 80%. In embodiments, knock-in efficiency of the population of engineered T cells is from 1% to 70%. In embodiments, knock-in efficiency of the population of engineered T cells is from 1% to 60%. In embodiments, knock-in efficiency of the population of engineered T cells is from 1% to 50%. In embodiments, knock-in efficiency of the population of engineered T cells is from 1% to 40%. In embodiments, knock-in efficiency of the population of engineered T cells is from 1% to 30%. In embodiments, knock-in efficiency of the population of engineered T cells is from 1% to 20%. In embodiments, knock-in efficiency of the population of engineered T cells is from 1% to 10%. In embodiments, knock-in efficiency of the population of engineered T cells is from 1% to 5%. Percent knock-in efficiency may be any value or subrange within the recited ranges, including endpoints. For the methods provided herein, in embodiments, the knock-in efficiency is about 60%. In embodiments, the knock-in efficiency is 60%.

Provided herein, inter alia, are methods for increasing expansion of a population of engineered T cells, including: (i) contacting a population of T cells with insulin, an insulin analog, an insulin agonist, an insulin partial agonist, and a polynucleotide, thereby forming the population of engineered T cells, and (ii) expanding the population of engineered T cells, thereby forming a population of expanded engineered T cells, wherein the insulin, insulin analog, insulin agonist, and/or insulin partial agonist increases the population of expanded engineered T cells relative to a population of engineered T cells that are not contacted with insulin, insulin analog, insulin agonist, and/or insulin partial agonist.

In embodiments, the contacting the population of T cells with the polynucleotide includes transfecting the population of T cells with the polynucleotide. In embodiments, the polynucleotide is a donor DNA. In embodiments, the polynucleotide includes: single-stranded DNA, double-stranded DNA, a linear DNA strand, a plasmid, a nanoplasmid, or a minicircle. In embodiments, the polynucleotide includes single-stranded DNA. In embodiments, the polynucleotide includes double-stranded DNA. In embodiments, the polynucleotide includes a linear DNA strand. In embodiments, the polynucleotide includes a plasmid. In embodiments, the polynucleotide includes a nanoplasmid. In embodiments, the polynucleotide includes a minicircle.

In embodiments, further including contacting the population of T cells with a gene editing reagent. In embodiments, contacting the population of T cells with the gene editing reagent includes transfecting the population of T cells with the gene editing reagent or a polynucleotide encoding the gene editing reagent. In embodiments, contacting the population of T cells with the gene editing reagent includes transfecting the population of T cells with the gene editing reagent. In embodiments, contacting the population of T cells with the gene editing reagent includes transfecting the population of T cells with a polynucleotide encoding the gene editing reagent. In embodiments, the population of T cells is transfected with the polynucleotide and the gene editing agent or the polynucleotide encoding the gene editing reagent simultaneously. In embodiments, the population of T cells is transfected with the polynucleotide and the gene editing agent simultaneously. In embodiments, the population of T cells is transfected with the polynucleotide and the polynucleotide encoding the gene editing reagent simultaneously.

In embodiments, the population of T cells is contacted with about 1 μg/ml to about 50 μg/ml of insulin, insulin analog, insulin agonist, and/or insulin partial agonist. In embodiments, the population of T cells is contacted with about 2 μg/ml to about 50 μg/ml of insulin, insulin analog, insulin agonist, and/or insulin partial agonist. In embodiments, the population of T cells is contacted with about 3 μg/ml to about 50 μg/ml of insulin, insulin analog, insulin agonist, and/or insulin partial agonist. In embodiments, the population of T cells is contacted with about 4 μg/ml to about 50 μg/ml of insulin, insulin analog, insulin agonist, and/or insulin partial agonist. In embodiments, the population of T cells is contacted with about 5 μg/ml to about 50 μg/ml of insulin, insulin analog, insulin agonist, and/or insulin partial agonist. In embodiments, the population of T cells is contacted with about 6 μg/ml to about 50 μg/ml of insulin, insulin analog, insulin agonist, and/or insulin partial agonist. In embodiments, the population of T cells is contacted with about 7 μg/ml to about 50 μg/ml of insulin, insulin analog, insulin agonist, and/or insulin partial agonist. In embodiments, the population of T cells is contacted with about 8 μg/ml to about 50 μg/ml of insulin, insulin analog, insulin agonist, and/or insulin partial agonist. In embodiments, the population of T cells is contacted with about 9 μg/ml to about 50 μg/ml of insulin, insulin analog, insulin agonist, and/or insulin partial agonist. In embodiments, the population of T cells is contacted with about 10 μg/ml to about 50 μg/ml of insulin, insulin analog, insulin agonist, and/or insulin partial agonist. In embodiments, the population of T cells is contacted with about 15 μg/ml to about 50 μg/ml of insulin, insulin analog, insulin agonist, and/or insulin partial agonist. In embodiments, the population of T cells is contacted with about 20 Vg/ml to about 50 μg/ml of insulin, insulin analog, insulin agonist, and/or insulin partial agonist. In embodiments, the population of T cells is contacted with about 25 μg/ml to about 50 μg/ml of insulin, insulin analog, insulin agonist, and/or insulin partial agonist. In embodiments, the population of T cells is contacted with about 30 μg/ml to about 50 μg/ml of insulin, insulin analog, insulin agonist, and/or insulin partial agonist. In embodiments, the population of T cells is contacted with about 35 μg/ml to about 50 μg/ml of insulin, insulin analog, insulin agonist, and/or insulin partial agonist. In embodiments, the population of T cells is contacted with about 40 μg/ml to about 50 μg/ml of insulin, insulin analog, insulin agonist, and/or insulin partial agonist. In embodiments, the population of T cells is contacted with about 45 μg/ml to about 50 μg/ml of insulin, insulin analog, insulin agonist, and/or insulin partial agonist.

In embodiments, the population of T cells is contacted with about 1 μg/ml to about 45 μg/ml of insulin, insulin analog, insulin agonist, and/or insulin partial agonist. In embodiments, the population of T cells is contacted with about 1 μg/ml to about 40 μg/ml of insulin, insulin analog, insulin agonist, and/or insulin partial agonist. In embodiments, the population of T cells is contacted with about 1 μg/ml to about 35 μg/ml of insulin, insulin analog, insulin agonist, and/or insulin partial agonist. In embodiments, the population of T cells is contacted with about 1 μg/ml to about 30 μg/ml of insulin, insulin analog, insulin agonist, and/or insulin partial agonist. In embodiments, the population of T cells is contacted with about 1 μg/ml to about 25 μg/ml of insulin, insulin analog, insulin agonist, and/or insulin partial agonist. In embodiments, the population of T cells is contacted with about 1 μg/ml to about 20 μg/ml of insulin, insulin analog, insulin agonist, and/or insulin partial agonist. In embodiments, the population of T cells is contacted with about 1 μg/ml to about 15 μg/ml of insulin, insulin analog, insulin agonist, and/or insulin partial agonist. In embodiments, the population of T cells is contacted with about 1 μg/ml to about 10 μg/ml of insulin, insulin analog, insulin agonist, and/or insulin partial agonist. In embodiments, the population of T cells is contacted with about 1 μg/ml to about 9 μg/ml of insulin, insulin analog, insulin agonist, and/or insulin partial agonist. In embodiments, the population of T cells is contacted with about 1 μg/ml to about 8 μg/ml of insulin, insulin analog, insulin agonist, and/or insulin partial agonist. In embodiments, the population of T cells is contacted with about 1 μg/ml to about 7 μg/ml of insulin, insulin analog, insulin agonist, and/or insulin partial agonist. In embodiments, the population of T cells is contacted with about 1 μg/ml to about 6 μg/ml of insulin, insulin analog, insulin agonist, and/or insulin partial agonist. In embodiments, the population of T cells is contacted with about 1 μg/ml to about 5 μg/ml of insulin, insulin analog, insulin agonist, and/or insulin partial agonist. In embodiments, the population of T cells is contacted with about 1 μg/ml to about 4 μg/ml of insulin, insulin analog, insulin agonist, and/or insulin partial agonist. In embodiments, the population of T cells is contacted with about 1 μg/ml to about 3 μg/ml of insulin, insulin analog, insulin agonist, and/or insulin partial agonist. In embodiments, the population of T cells is contacted with about 1 μg/ml to about 2 μg/ml of insulin, insulin analog, insulin agonist, and/or insulin partial agonist.

In embodiments, the population of T cells is contacted with 1 μg/ml to 50 μg/ml of insulin, insulin analog, insulin agonist, and/or insulin partial agonist. In embodiments, the population of T cells is contacted with 2 μg/ml to 50 μg/ml of insulin, insulin analog, insulin agonist, and/or insulin partial agonist. In embodiments, the population of T cells is contacted with 3 μg/ml to 50 μg/ml of insulin, insulin analog, insulin agonist, and/or insulin partial agonist. In embodiments, the population of T cells is contacted with 4 μg/ml to 50 μg/ml of insulin, insulin analog, insulin agonist, and/or insulin partial agonist. In embodiments, the population of T cells is contacted with 5 μg/ml to 50 μg/ml of insulin, insulin analog, insulin agonist, and/or insulin partial agonist. In embodiments, the population of T cells is contacted with 6 μg/ml to 50 μg/ml of insulin, insulin analog, insulin agonist, and/or insulin partial agonist. In embodiments, the population of T cells is contacted with 7 μg/ml to 50 μg/ml of insulin, insulin analog, insulin agonist, and/or insulin partial agonist. In embodiments, the population of T cells is contacted with 8 μg/ml to 50 μg/ml of insulin, insulin analog, insulin agonist, and/or insulin partial agonist. In embodiments, the population of T cells is contacted with 9 μg/ml to 50 μg/ml of insulin, insulin analog, insulin agonist, and/or insulin partial agonist. In embodiments, the population of T cells is contacted with 10 μg/ml to 50 μg/ml of insulin, insulin analog, insulin agonist, and/or insulin partial agonist. In embodiments, the population of T cells is contacted with 15 μg/ml to 50 μg/ml of insulin, insulin analog, insulin agonist, and/or insulin partial agonist. In embodiments, the population of T cells is contacted with 20 μg/ml to 50 μg/ml of insulin, insulin analog, insulin agonist, and/or insulin partial agonist. In embodiments, the population of T cells is contacted with 25 μg/ml to 50 μg/ml of insulin, insulin analog, insulin agonist, and/or insulin partial agonist. In embodiments, the population of T cells is contacted with 30 μg/ml to 50 μg/ml of insulin, insulin analog, insulin agonist, and/or insulin partial agonist. In embodiments, the population of T cells is contacted with 35 μg/ml to 50 μg/ml of insulin, insulin analog, insulin agonist, and/or insulin partial agonist. In embodiments, the population of T cells is contacted with 40 μg/ml to 50 μg/ml of insulin, insulin analog, insulin agonist, and/or insulin partial agonist. In embodiments, the population of T cells is contacted with 45 μg/ml to 50 μg/ml of insulin, insulin analog, insulin agonist, and/or insulin partial agonist.

In embodiments, the population of T cells is contacted with 1 μg/ml to 45 μg/ml of insulin, insulin analog, insulin agonist, and/or insulin partial agonist. In embodiments, the population of T cells is contacted with 1 μg/ml to 40 μg/ml of insulin, insulin analog, insulin agonist, and/or insulin partial agonist. In embodiments, the population of T cells is contacted with 1 μg/ml to 35 μg/ml of insulin, insulin analog, insulin agonist, and/or insulin partial agonist. In embodiments, the population of T cells is contacted with 1 μg/ml to 30 μg/ml of insulin, insulin analog, insulin agonist, and/or insulin partial agonist. In embodiments, the population of T cells is contacted with 1 μg/ml to 25 μg/ml of insulin, insulin analog, insulin agonist, and/or insulin partial agonist. In embodiments, the population of T cells is contacted with 1 μg/ml to 20 μg/ml of insulin, insulin analog, insulin agonist, and/or insulin partial agonist. In embodiments, the population of T cells is contacted with 1 μg/ml to 15 μg/ml of insulin, insulin analog, insulin agonist, and/or insulin partial agonist. In embodiments, the population of T cells is contacted with 1 μg/ml to 10 μg/ml of insulin, insulin analog, insulin agonist, and/or insulin partial agonist. In embodiments, the population of T cells is contacted with 1 μg/ml to 9 μg/ml of insulin, insulin analog, insulin agonist, and/or insulin partial agonist. In embodiments, the population of T cells is contacted with 1 μg/ml to 8 μg/ml of insulin, insulin analog, insulin agonist, and/or insulin partial agonist. In embodiments, the population of T cells is contacted with 1 μg/ml to 7 μg/ml of insulin, insulin analog, insulin agonist, and/or insulin partial agonist. In embodiments, the population of T cells is contacted with 1 μg/ml to 6 μg/ml of insulin, insulin analog, insulin agonist, and/or insulin partial agonist. In embodiments, the population of T cells is contacted with 1 μg/ml to 5 μg/ml of insulin, insulin analog, insulin agonist, and/or insulin partial agonist. In embodiments, the population of T cells is contacted with 1 μg/ml to 4 μg/ml of insulin, insulin analog, insulin agonist, and/or insulin partial agonist. In embodiments, the population of T cells is contacted with 1 μg/ml to 3 μg/ml of insulin, insulin analog, insulin agonist, and/or insulin partial agonist. In embodiments, the population of T cells is contacted with 1 μg/ml to 2 μg/ml of insulin, insulin analog, insulin agonist, and/or insulin partial agonist. Concentration may be any value or subrange within the recited ranges, including endpoints.

In embodiments, the population of T cells is contacted simultaneously with the polynucleotide and insulin, insulin analog, insulin agonist, and/or insulin partial agonist. In embodiments, the population of T cells is contacted sequentially with the polynucleotide and insulin, insulin analog, insulin agonist, and/or insulin partial agonist. In embodiments, the population of T cells is contacted with insulin, insulin analog, insulin agonist, and/or insulin partial agonist prior to contacting with the polynucleotide. In embodiments, the population of T cells is contacted with insulin, insulin analog, insulin agonist, and/or insulin partial agonist after contacting with the polynucleotide. In embodiments, the population of T cells is contacted with insulin, insulin analog, insulin agonist, and/or insulin partial agonist prior to and after contacting with the polynucleotide.

In embodiments, the population of expanded engineered T cells is increased from at least about 0.1 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.2 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.3 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.4 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.5 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.6 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.7 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.8 fold to at least about 5.0 fold relative to a population of engineered T cells that are not contacted with insulin, insulin analog, insulin agonist, and/or insulin partial agonist. In embodiments, the population of expanded engineered T cells is increased from at least about 0.9 fold to at least about 5.0 fold relative to a population of engineered T cells that are not contacted with insulin, insulin analog, insulin agonist, and/or insulin partial agonist. In embodiments, the population of expanded engineered T cells is increased from at least about 1.0 fold to at least about 5.0 fold relative to a population of engineered T cells that are not contacted with insulin, insulin analog, insulin agonist, and/or insulin partial agonist. In embodiments, the population of expanded engineered T cells is increased from at least about 1.5 fold to at least about 5.0 fold relative to a population of engineered T cells that are not contacted with insulin, insulin analog, insulin agonist, and/or insulin partial agonist. In embodiments, the population of expanded engineered T cells is increased from at least about 2.0 fold to at least about 5.0 fold relative to a population of engineered T cells that are not contacted with insulin, insulin analog, insulin agonist, and/or insulin partial agonist. In embodiments, the population of expanded engineered T cells is increased from at least about 2.5 fold to at least about 5.0 fold relative to a population of engineered T cells that are not contacted with insulin, insulin analog, insulin agonist, and/or insulin partial agonist. In embodiments, the population of expanded engineered T cells is increased from at least about 3.0 fold to at least about 5.0 fold relative to a population of engineered T cells that are not contacted with insulin, insulin analog, insulin agonist, and/or insulin partial agonist. In embodiments, the population of expanded engineered T cells is increased from at least about 3.5 fold to at least about 5.0 fold relative to a population of engineered T cells that are not contacted with insulin, insulin analog, insulin agonist, and/or insulin partial agonist. In embodiments, the population of expanded engineered T cells is increased from at least about 4.0 fold to at least about 5.0 fold relative to a population of engineered T cells that are not contacted with insulin, insulin analog, insulin agonist, and/or insulin partial agonist. In embodiments, the population of expanded engineered T cells is increased from at least about 4.5 fold to at least about 5.0 fold relative to a population of engineered T cells that are not contacted with insulin, insulin analog, insulin agonist, and/or insulin partial agonist.

In embodiments, the population of expanded engineered T cells is increased from at least about 0.1 fold to at least about 4.5 fold relative to a population of engineered T cells that are not contacted with insulin, insulin analog, insulin agonist, and/or insulin partial agonist. In embodiments, the population of expanded engineered T cells is increased from at least about 0.1 fold to at least about 4.0 fold relative to a population of engineered T cells that are not contacted with insulin, insulin analog, insulin agonist, and/or insulin partial agonist. In embodiments, the population of expanded engineered T cells is increased from at least about 0.1 fold to at least about 3.5 fold relative to a population of engineered T cells that are not contacted with insulin, insulin analog, insulin agonist, and/or insulin partial agonist. In embodiments, the population of expanded engineered T cells is increased from at least about 0.1 fold to at least about 3.0 fold relative to a population of engineered T cells that are not contacted with insulin, insulin analog, insulin agonist, and/or insulin partial agonist. In embodiments, the population of expanded engineered T cells is increased from at least about 0.1 fold to at least about 2.5 fold relative to a population of engineered T cells that are not contacted with insulin, insulin analog, insulin agonist, and/or insulin partial agonist. In embodiments, the population of expanded engineered T cells is increased from at least about 0.1 fold to at least about 2.0 fold relative to a population of engineered T cells that are not contacted with insulin, insulin analog, insulin agonist, and/or insulin partial agonist. In embodiments, the population of expanded engineered T cells is increased from at least about 0.1 fold to at least about 1.5 fold relative to a population of engineered T cells that are not contacted with insulin, insulin analog, insulin agonist, and/or insulin partial agonist. In embodiments, the population of expanded engineered T cells is increased from at least about 0.1 fold to at least about 1.0 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.9 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.8 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.7 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.6 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.5 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.4 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.3 fold relative to a population of engineered T cells that are not contacted with insulin, insulin analog, insulin agonist, and/or insulin partial agonist. In embodiments, the population of expanded engineered T cells is increased from at least about 0.1 fold to at least about 0.2 fold relative to a population of engineered T cells that are not contacted with insulin, insulin analog, insulin agonist, and/or insulin partial agonist.

In embodiments, the population of expanded engineered T cells is increased from at least 0.1 fold to at least 5.0 fold relative to a population of engineered T cells that are not contacted with insulin, insulin analog, insulin agonist, and/or insulin partial agonist. In embodiments, the population of expanded engineered T cells is increased from at least 0.2 fold to at least 5.0 fold relative to a population of engineered T cells that are not contacted with insulin, insulin analog, insulin agonist, and/or insulin partial agonist. In embodiments, the population of expanded engineered T cells is increased from at least 0.3 fold to at least 5.0 fold relative to a population of engineered T cells that are not contacted with insulin, insulin analog, insulin agonist, and/or insulin partial agonist. In embodiments, the population of expanded engineered T cells is increased from at least 0.4 fold to at least 5.0 fold relative to a population of engineered T cells that are not contacted with insulin, insulin analog, insulin agonist, and/or insulin partial agonist. In embodiments, the population of expanded engineered T cells is increased from at least 0.5 fold to at least 5.0 fold relative to a population of engineered T cells that are not contacted with insulin, insulin analog, insulin agonist, and/or insulin partial agonist. In embodiments, the population of expanded engineered T cells is increased from at least 0.6 fold to at least 5.0 fold relative to a population of engineered T cells that are not contacted with insulin, insulin analog, insulin agonist, and/or insulin partial agonist. In embodiments, the population of expanded engineered T cells is increased from at least 0.7 fold to at least 5.0 fold relative to a population of engineered T cells that are not contacted with insulin, insulin analog, insulin agonist, and/or insulin partial agonist. In embodiments, the population of expanded engineered T cells is increased from at least 0.8 fold to at least 5.0 fold relative to a population of engineered T cells that are not contacted with insulin, insulin analog, insulin agonist, and/or insulin partial agonist. In embodiments, the population of expanded engineered T cells is increased from at least 0.9 fold to at least 5.0 fold relative to a population of engineered T cells that are not contacted with insulin, insulin analog, insulin agonist, and/or insulin partial agonist. In embodiments, the population of expanded engineered T cells is increased from at least 1.0 fold to at least 5.0 fold relative to a population of engineered T cells that are not contacted with insulin, insulin analog, insulin agonist, and/or insulin partial agonist. In embodiments, the population of expanded engineered T cells is increased from at least 1.5 fold to at least 5.0 fold relative to a population of engineered T cells that are not contacted with insulin, insulin analog, insulin agonist, and/or insulin partial agonist. In embodiments, the population of expanded engineered T cells is increased from at least 2.0 fold to at least 5.0 fold relative to a population of engineered T cells that are not contacted with insulin, insulin analog, insulin agonist, and/or insulin partial agonist. In embodiments, the population of expanded engineered T cells is increased from at least 2.5 fold to at least 5.0 fold relative to a population of engineered T cells that are not contacted with insulin, insulin analog, insulin agonist, and/or insulin partial agonist. In embodiments, the population of expanded engineered T cells is increased from at least 3.0 fold to at least 5.0 fold relative to a population of engineered T cells that are not contacted with insulin, insulin analog, insulin agonist, and/or insulin partial agonist. In embodiments, the population of expanded engineered T cells is increased from at least 3.5 fold to at least 5.0 fold relative to a population of engineered T cells that are not contacted with insulin, insulin analog, insulin agonist, and/or insulin partial agonist. In embodiments, the population of expanded engineered T cells is increased from at least 4.0 fold to at least 5.0 fold relative to a population of engineered T cells that are not contacted with insulin, insulin analog, insulin agonist, and/or insulin partial agonist. In embodiments, the population of expanded engineered T cells is increased from at least 4.5 fold to at least 5.0 fold relative to a population of engineered T cells that are not contacted with insulin, insulin analog, insulin agonist, and/or insulin partial agonist.

In embodiments, the population of expanded engineered T cells is increased from at least 0.1 fold to at least 4.5 fold relative to a population of engineered T cells that are not contacted with insulin, insulin analog, insulin agonist, and/or insulin partial agonist. In embodiments, the population of expanded engineered T cells is increased from at least 0.1 fold to at least 4.0 fold relative to a population of engineered T cells that are not contacted with insulin, insulin analog, insulin agonist, and/or insulin partial agonist. In embodiments, the population of expanded engineered T cells is increased from at least 0.1 fold to at least 3.5 fold relative to a population of engineered T cells that are not contacted with insulin, insulin analog, insulin agonist, and/or insulin partial agonist. In embodiments, the population of expanded engineered T cells is increased from at least 0.1 fold to at least 3.0 fold relative to a population of engineered T cells that are not contacted with insulin, insulin analog, insulin agonist, and/or insulin partial agonist. In embodiments, the population of expanded engineered T cells is increased from at least 0.1 fold to at least 2.5 fold relative to a population of engineered T cells that are not contacted with insulin, insulin analog, insulin agonist, and/or insulin partial agonist. In embodiments, the population of expanded engineered T cells is increased from at least 0.1 fold to at least 2.0 fold relative to a population of engineered T cells that are not contacted with insulin, insulin analog, insulin agonist, and/or insulin partial agonist. In embodiments, the population of expanded engineered T cells is increased from at least 0.1 fold to at least 1.5 fold relative to a population of engineered T cells that are not contacted with insulin, insulin analog, insulin agonist, and/or insulin partial agonist. In embodiments, the population of expanded engineered T cells is increased from at least 0.1 fold to at least 1.0 fold relative to a population of engineered T cells that are not contacted with insulin, insulin analog, insulin agonist, and/or insulin partial agonist. In embodiments, the population of expanded engineered T cells is increased from at least 0.1 fold to at least 0.9 fold relative to a population of engineered T cells that are not contacted with insulin, insulin analog, insulin agonist, and/or insulin partial agonist. In embodiments, the population of expanded engineered T cells is increased from at least 0.1 fold to at least 0.8 fold relative to a population of engineered T cells that are not contacted with insulin, insulin analog, insulin agonist, and/or insulin partial agonist. In embodiments, the population of expanded engineered T cells is increased from at least 0.1 fold to at least 0.7 fold relative to a population of engineered T cells that are not contacted with insulin, insulin analog, insulin agonist, and/or insulin partial agonist. In embodiments, the population of expanded engineered T cells is increased from at least 0.1 fold to at least 0.6 fold relative to a population of engineered T cells that are not contacted with insulin, insulin analog, insulin agonist, and/or insulin partial agonist. In embodiments, the population of expanded engineered T cells is increased from at least 0.1 fold to at least 0.5 fold relative to a population of engineered T cells that are not contacted with insulin, insulin analog, insulin agonist, and/or insulin partial agonist. In embodiments, the population of expanded engineered T cells is increased from at least 0.1 fold to at least 0.4 fold relative to a population of engineered T cells that are not contacted with insulin, insulin analog, insulin agonist, and/or insulin partial agonist. In embodiments, the population of expanded engineered T cells is increased from at least 0.1 fold to at least 0.3 fold relative to a population of engineered T cells that are not contacted with insulin, insulin analog, insulin agonist, and/or insulin partial agonist. In embodiments, the population of expanded engineered T cells is increased from at least 0.1 fold to at least 0.2 fold relative to a population of engineered T cells that are not contacted with insulin, insulin analog, insulin agonist, and/or insulin partial agonist. For the methods provided herein, in embodiments, the population of expanded engineered T cells is increased from about 2.0 fold to about 3.0 fold. In embodiments, the population of expanded engineered T cells is increased from 2.0 fold to 3.0 fold. Fold increase may be any value or subrange within the recited ranges, including endpoints.

In embodiments, the population of engineered T cells are expanded from at least about 0.1 fold to at least about 1000 fold. In embodiments, the population of engineered T cells are expanded from at least about 0.5 fold to at least about 1000 fold. In embodiments, the population of engineered T cells are expanded from at least about 1.0 fold to at least about 1000 fold. In embodiments, the population of engineered T cells are expanded from at least about 5.0 fold to at least about 1000 fold. In embodiments, the population of engineered T cells are expanded from at least about 10 fold to at least about 1000 fold. In embodiments, the population of engineered T cells are expanded from at least about 20 fold to at least about 1000 fold. In embodiments, the population of engineered T cells are expanded from at least about 30 fold to at least about 1000 fold. In embodiments, the population of engineered T cells are expanded from at least about 40 fold to at least about 1000 fold. In embodiments, the population of engineered T cells are expanded from at least about 50 fold to at least about 1000 fold. In embodiments, the population of engineered T cells are expanded from at least about 100 fold to at least about 1000 fold. In embodiments, the population of engineered T cells are expanded from at least about 200 fold to at least about 1000 fold. In embodiments, the population of engineered T cells are expanded from at least about 300 fold to at least about 1000 fold. In embodiments, the population of engineered T cells are expanded from at least about 400 fold to at least about 1000 fold. In embodiments, the population of engineered T cells are expanded from at least about 500 fold to at least about 1000 fold. In embodiments, the population of engineered T cells are expanded from at least about 600 fold to at least about 1000 fold. In embodiments, the population of engineered T cells are expanded from at least about 700 fold to at least about 1000 fold. In embodiments, the population of engineered T cells are expanded from at least about 800 fold to at least about 1000 fold. In embodiments, the population of engineered T cells are expanded from at least about 900 fold to at least about 1000 fold.

In embodiments, the population of engineered T cells are expanded from at least about 0.1 fold to at least about 900 fold. In embodiments, the population of engineered T cells are expanded from at least about 0.1 fold to at least about 800 fold. In embodiments, the population of engineered T cells are expanded from at least about 0.1 fold to at least about 700 fold. In embodiments, the population of engineered T cells are expanded from at least about 0.1 fold to at least about 600 fold. In embodiments, the population of engineered T cells are expanded from at least about 0.1 fold to at least about 500 fold. In embodiments, the population of engineered T cells are expanded from at least about 0.1 fold to at least about 400 fold. In embodiments, the population of engineered T cells are expanded from at least about 0.1 fold to at least about 300 fold. In embodiments, the population of engineered T cells are expanded from at least about 0.1 fold to at least about 200 fold. In embodiments, the population of engineered T cells are expanded from at least about 0.1 fold to at least about 100 fold. In embodiments, the population of engineered T cells are expanded from at least about 0.1 fold to at least about 50 fold. In embodiments, the population of engineered T cells are expanded from at least about 0.1 fold to at least about 40 fold. In embodiments, the population of engineered T cells are expanded from at least about 0.1 fold to at least about 30 fold. In embodiments, the population of engineered T cells are expanded from at least about 0.1 fold to at least about 20 fold. In embodiments, the population of engineered T cells are expanded from at least about 0.1 fold to at least about 10 fold. In embodiments, the population of engineered T cells are expanded from at least about 0.1 fold to at least about 5.0 fold. In embodiments, the population of engineered T cells are expanded from at least about 0.1 fold to at least about 1.0 fold. In embodiments, the population of engineered T cells are expanded from at least about 0.1 fold to at least about 0.5 fold.

In embodiments, the population of engineered T cells are expanded from at least 0.1 fold to at least 1000 fold. In embodiments, the population of engineered T cells are expanded from at least 0.5 fold to at least 1000 fold. In embodiments, the population of engineered T cells are expanded from at least 1.0 fold to at least 1000 fold. In embodiments, the population of engineered T cells are expanded from at least 5.0 fold to at least 1000 fold. In embodiments, the population of engineered T cells are expanded from at least 10 fold to at least 1000 fold. In embodiments, the population of engineered T cells are expanded from at least 20 fold to at least 1000 fold. In embodiments, the population of engineered T cells are expanded from at least 30 fold to at least 1000 fold. In embodiments, the population of engineered T cells are expanded from at least 40 fold to at least 1000 fold. In embodiments, the population of engineered T cells are expanded from at least 50 fold to at least 1000 fold. In embodiments, the population of engineered T cells are expanded from at least 100 fold to at least 1000 fold. In embodiments, the population of engineered T cells are expanded from at least 200 fold to at least 1000 fold. In embodiments, the population of engineered T cells are expanded from at least 300 fold to at least 1000 fold. In embodiments, the population of engineered T cells are expanded from at least 400 fold to at least 1000 fold. In embodiments, the population of engineered T cells are expanded from at least 500 fold to at least 1000 fold. In embodiments, the population of engineered T cells are expanded from at least 600 fold to at least 1000 fold. In embodiments, the population of engineered T cells are expanded from at least 700 fold to at least 1000 fold. In embodiments, the population of engineered T cells are expanded from at least 800 fold to at least 1000 fold. In embodiments, the population of engineered T cells are expanded from at least 900 fold to at least 1000 fold.

In embodiments, the population of engineered T cells are expanded from at least 0.1 fold to at least 900 fold. In embodiments, the population of engineered T cells are expanded from at least 0.1 fold to at least 800 fold. In embodiments, the population of engineered T cells are expanded from at least 0.1 fold to at least 700 fold. In embodiments, the population of engineered T cells are expanded from at least 0.1 fold to at least 600 fold. In embodiments, the population of engineered T cells are expanded from at least 0.1 fold to at least 500 fold. In embodiments, the population of engineered T cells are expanded from at least 0.1 fold to at least 400 fold. In embodiments, the population of engineered T cells are expanded from at least 0.1 fold to at least 300 fold. In embodiments, the population of engineered T cells are expanded from at least 0.1 fold to at least 200 fold. In embodiments, the population of engineered T cells are expanded from at least 0.1 fold to at least 100 fold. In embodiments, the population of engineered T cells are expanded from at 0.1 fold to at least 50 fold. In embodiments, the population of engineered T cells are expanded from at least 0.1 fold to at least 40 fold. In embodiments, the population of engineered T cells are expanded from at least 0.1 fold to at least 30 fold. In embodiments, the population of engineered T cells are expanded from at least 0.1 fold to at least 20 fold. In embodiments, the population of engineered T cells are expanded from at least 0.1 fold to at least 10 fold. In embodiments, the population of engineered T cells are expanded from at least 0.1 fold to at least 5.0 fold. In embodiments, the population of engineered T cells are expanded from at least 0.1 fold to at least 1.0 fold. In embodiments, the population of engineered T cells are expanded from at least 0.1 fold to at least 0.5 fold. Fold increase may be any value or subrange within the recited ranges, including endpoints. For the methods provided herein, in embodiments, the engineered T cells are expanded about 20 fold. In embodiments, the engineered T cells are expanded 20 fold.

In embodiments, the methods disclosed herein including embodiments thereof are performed under Good Manufacturing Practices (GMP).

### Engineered T Cell Compositions

Provided herein, inter alia, are compositions including a population of engineered T cells made by a method provided herein including embodiments thereof. The population of engineered T cells may have increased viability and/or expansion compared to a population of engineered T cells made by a method wherein a population T cells are not contacted with insulin, an insulin analog, an insulin agonist, and/or an insulin partial agonist prior to generation of the population of engineered T cells. Thus, in an aspect is provided a population of engineered T cells made by contacting a population of T cells with a polynucleotide and insulin, an insulin analog, an insulin agonist, and/or an insulin partial agonist.

In embodiments, the population of T cells and the polynucleotide are contacted in the presence of insulin, an insulin analog, an insulin agonist, and/or an insulin partial agonist. In embodiments, the population of T cells is contacted sequentially with the polynucleotide and insulin, an insulin analog, an insulin agonist, and/or an insulin partial agonist. In embodiments, the population of T cells is contacted with insulin, an insulin analog, an insulin agonist, and/or an insulin partial agonist prior to the polynucleotide. In embodiments, the population of T cells is contacted with insulin, an insulin analog, an insulin agonist, and/or an insulin partial agonist concurrently the polynucleotide. In embodiments, the population of T cells is contacted with insulin, an insulin analog, an insulin agonist, and/or an insulin partial agonist after the polynucleotide.

In embodiments, the population of T cells is cultured with insulin, an insulin analog, an insulin agonist, and/or an insulin partial agonist for about 30 minutes to about 48 hours. In embodiments, the population of T cells is cultured with insulin, an insulin analog, an insulin agonist, and/or an insulin partial agonist for about 1 hour to about 48 hours. In embodiments, the population of T cells is cultured with insulin, an insulin analog, an insulin agonist, and/or an insulin partial agonist for about 2 hours to about 48 hours. In embodiments, the population of T cells is cultured with insulin, an insulin analog, an insulin agonist, and/or an insulin partial agonist for about 4 hours to about 48 hours. In embodiments, the population of T cells is cultured with insulin, an insulin analog, an insulin agonist, and/or an insulin partial agonist for about 6 hours to about 48 hours. In embodiments, the population of T cells is cultured with insulin, an insulin analog, an insulin agonist, and/or an insulin partial agonist for about 12 hours to about 48 hours. In embodiments, the population of T cells is cultured with insulin, an insulin analog, an insulin agonist, and/or an insulin partial agonist for about 24 hours to about 48 hours.

In embodiments, the population of T cells is cultured with insulin, an insulin analog, an insulin agonist, and/or an insulin partial agonist for about 30 minutes to about 24 hours. In embodiments, the population of T cells is cultured with insulin, an insulin analog, an insulin agonist, and/or an insulin partial agonist for about 30 minutes to about 12 hours. In embodiments, the population of T cells is cultured with insulin, an insulin analog, an insulin agonist, and/or an insulin partial agonist for about 30 minutes to about 6 hours. In embodiments, the population of T cells is cultured with insulin, an insulin analog, an insulin agonist, and/or an insulin partial agonist for about 30 minutes to about 4 hours. In embodiments, the population of T cells is cultured with insulin, an insulin analog, an insulin agonist, and/or an insulin partial agonist for about 30 minutes to about 2 hours. In embodiments, the population of T cells is cultured with insulin, an insulin analog, an insulin agonist, and/or an insulin partial agonist for about 30 minutes to about 1 hour.

In embodiments, the population of T cells is cultured with insulin, an insulin analog, an insulin agonist, and/or an insulin partial agonist for about 30 minutes. In embodiments, the population of T cells is cultured with insulin, an insulin analog, an insulin agonist, and/or an insulin partial agonist for about 1 hour. In embodiments, the population of T cells is cultured with insulin, an insulin analog, an insulin agonist, and/or an insulin partial agonist for about 2 hours. In embodiments, the population of T cells is cultured with insulin, an insulin analog, an insulin agonist, and/or an insulin partial agonist for about 4 hours. In embodiments, the population of T cells is cultured with insulin, an insulin analog, an insulin agonist, and/or an insulin partial agonist for about 6 hours. In embodiments, the population of T cells is cultured with insulin, an insulin analog, an insulin agonist, and/or an insulin partial agonist for about 12 hours. In embodiments, the population of T cells is cultured with insulin, an insulin analog, an insulin agonist, and/or an insulin partial agonist for about 24 hours. In embodiments, the population of T cells is cultured with insulin, insulin analog, insulin agonist, and/or insulin partial agonist for about 48 hours.

### T Cell Compositions

Provided herein are compositions including a population of T cells and insulin, an insulin analog, an insulin agonist, and/or an insulin partial agonist, wherein the compositions are useful for generating a population of engineered T cells. Applicant has demonstrated that insulin increases gene editing efficiency in the T cells. Applicant has further demonstrated that the compositions provided herein including embodiments thereof result in a population of engineered T cells with increased cell viability and expansion compared to compositions that do not include insulin. Thus, in an aspect is provided a composition including a population of T cells, a polynucleotide, and insulin, an insulin analog, an insulin agonist, and/or an insulin partial agonist. In embodiments, the population of T cells includes engineered T cells. In embodiments, the engineered T cells are engineered as described herein.

In embodiments, the composition further includes a gene editing reagent.

### Pharmaceutical Compositions

The compositions provided herein, including T cell compositions and engineered T cell compositions, are contemplated to be effective for treating diseases (e.g. cancer). For example, the engineered T cells provided herein may include exogenous T cell receptors specific for cancer cell antigens. Thus, in an aspect is provided a pharmaceutical composition including the engineered T cell provided herein including embodiments thereof. In embodiments, the pharmaceutical composition further includes a pharmaceutically acceptable excipient.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

### Methods of Treatment

The engineered T cells provided herein including embodiments thereof are contemplated to be specific for disease-associated antigens (e.g. cancer cell antigens), thereby allowing effective targeting of cancer cells. Engineered T cells may include, for example, one or more exogenous T cell receptors engineered to be specific for an individual's cancer cells, allowing personalized and specific targeting of the cancer cells. Thus, in an aspect is provided a method of treating a disease in a subject in need thereof, including administering a therapeutically effective amount of the engineered T cell provided herein including embodiments thereof or the pharmaceutical composition provided herein including embodiments thereof. In embodiments, the method includes administering a therapeutically effective amount of the engineered T cell provided herein including embodiments thereof. In embodiments, the method includes administering a therapeutically effective amount of the pharmaceutical composition provided herein including embodiments thereof.

For the methods provided herein, in embodiments, the engineered T cell may be generated from the subject. For example, a T cell may be extracted from the subject, contacted with a polynucleotide (e.g. donor nucleic acid) and insulin ex vivo, thereby generating an engineered T cell, and administered back to the subject. Thus, in embodiments, the engineered T cell is an autologous T cell. In embodiments, the engineered T cell may be generated from T cells that are not taken from the subject. For example, the engineered T cell may be generated from a healthy subject (e.g. a subject who does not have cancer). Thus, in embodiments, the engineered T cell is an allogeneic T cell.

For the methods provided herein, in embodiments, the disease is cancer. In embodiments, the cancer is melanoma, lymphoma or leukemia. In embodiments, the cancer is melanoma. In embodiments, the cancer is lymphoma. In embodiments, the cancer is leukemia.

In embodiments, the cancer is leukemia, lymphoma, a carcinoma, a sarcoma, brain cancer, glioma, glioblastoma, neuroblastoma, prostate cancer, colorectral cancer, pancreatic cancer, medulloblastoma, melanoma, cervical cancer, gastric cancer, ovarian cancer, lung cancer, cancer of the head and neck, breast cancer, liver cancer, or uterine cancer. In embodiments, the cancer is a carcinoma. In embodiments the cancer is a sarcoma. In embodiments, the cancer is brain cancer. In embodiments, the cancer is glioma. In embodiments, the cancer is glioblastoma. In embodiments, the cancer is neuroblastoma. In embodiments, the cancer is prostate cancer. In embodiments, the cancer is colorectral cancer. In embodiments, the cancer is pancreatic cancer. In embodiments, the cancer is medulloblastoma. In embodiments, the cancer is cervical cancer. In embodiments, the cancer is gastric cancer. In embodiments, the cancer is ovarian cancer. In embodiments, the cancer is lung cancer. In embodiments, the cancer is cancer of the head and neck. In embodiments, the cancer is breast cancer. In embodiments, the cancer is liver cancer. In embodiments, the cancer is uterine cancer.

### EXAMPLES

### Example 1: Insulin Treatment Drastically Improves T Cell Engineering Efficiency and Expansion

Use of electroporation for transgene delivery allows a virus-free, convenient, efficient and safe approach for cell engineering. Compared to the use of recombinant viral vectors, which has been widely used in the cell therapy space, utilizing electroporation for genome editing saves time and is manufacturing friendly, as well as enabling a more precise genome targeting approach when using CRISPR/Cas9 for gene editing. However, electroporation can be harsh on cells, leading to poor cell viability and low recovery rates post transfection. Hence, an effective, safe and manufacturing friendly approach to efficiently improve T cell engineering, cell expansion, and total edited T cell (TEC) numbers during T cell receptor (TCR) engineering process via electroporation is desirable. Here we used Wilms tumor gene 1 (WT1) peptide-associated T cell receptor (TCR), which offers a clinically relevant system, as a DNA template to test the impact of our engineering process on the derived T cells as the final drug product (FDP). Our findings revealed that Akt and ERK signaling pathways are activated during the T cell engineering process and their activation correlated with the T cell culture viability and expansion. Further activation of these signaling pathways by addition of insulin to the cultured T cells effectively enhanced T cell expansion, total edited cell number (TEC) and TCR engineering efficiency of the FDP, contributing to up to 4-fold improvement in cell expansion and TEC, as well as 2-fold enhancement of knock-in efficiency. Without being bound by theory, these findings suggest that the addition of insulin to the T cell culture during the T cell engineering process may improve mitochondrial membrane potential and hence mitochondrial function, enhance T cell metabolism, and/or attenuate apoptosis. All these enhancements may be due to activation of both Akt and ERK signaling pathways, which was observed when T cells were pre-treated with insulin prior to transfection. Of note, in our studies T cell editing efficiency in the FDP was also improved upon pre-treatment of T cells with insulin prior to transfection, compared to the control groups. Furthermore, no significant difference in phenotype or T cell functionality between insulin pre-treatment groups versus control groups were observed, indicating that insulin pre-treatment of T cells can be a safe and translationally relevant approach for T cell engineering processes.

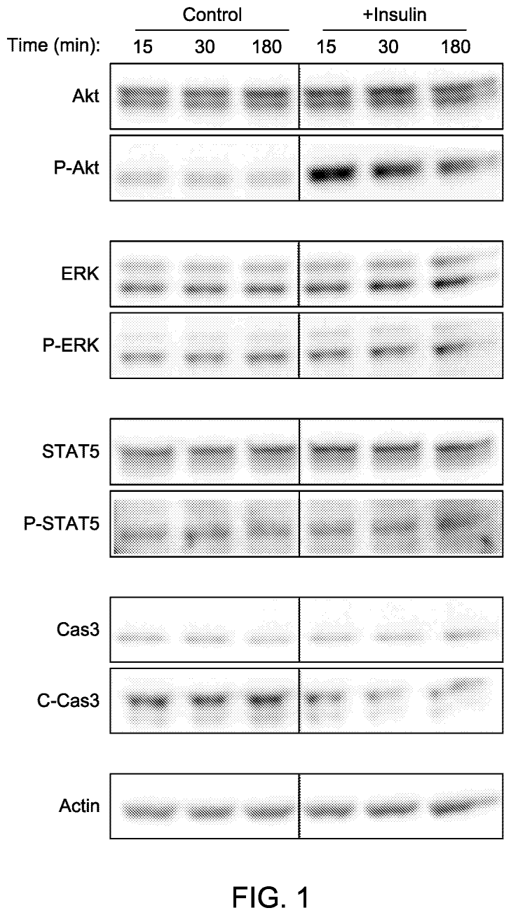

Insulin treatment stimulates Akt signaling pathway: Recently it has been reported that the insulin receptor plays a crucial role in T cell immunity in vivo. In numerous studies insulin has been shown to stimulate cell growth in many different cell types. However, its potential in T cell engineering and manufacturing process was previously unknown. Our initial analysis of activated cell signaling pathways during the T cell engineering process revealed that both Akt and Erk signaling pathways may be activated during the T cell engineering process (FIG. 1).

To confirm that the addition of insulin to T cell cultures during the engineering process can positively impact T cell growth, Naïve human CD8+T cells were isolated and stimulated by antiCD3/CD28 antibodies for 2 days followed by starvation for 4 hours (h) by culturing them in phosphate buffered saline (PBS). Cells were then stimulated by addition of insulin to the culture and samples were collected as indicated (FIG. 1). Western blot data revealed that insulin induced phosphorylation of AKT in human CD8+ T cells (FIG. 1) as early as 15 min post insulin treatment and AKT phosphorylation levels started to decline by 3 h post treatment (FIG. 1). Additionally, cleaved-caspase 3 (C-Cas3) levels were significantly reduced upon addition of insulin, suggesting that insulin treatment may also attenuate T cell apoptosis process, as shown in FIG. 1. No significant increase in the phosphorylation of ERK1/2 or STAT5 were observed upon stimulating starved cells with insulin, suggesting that addition of insulin to the activated CD8+ T cells culture media initially triggers AKT signaling. However, potential impact of insulin treatment at later time points on Ras-Raf-MEK-Erk signaling pathway in cultured T cells was not determined.

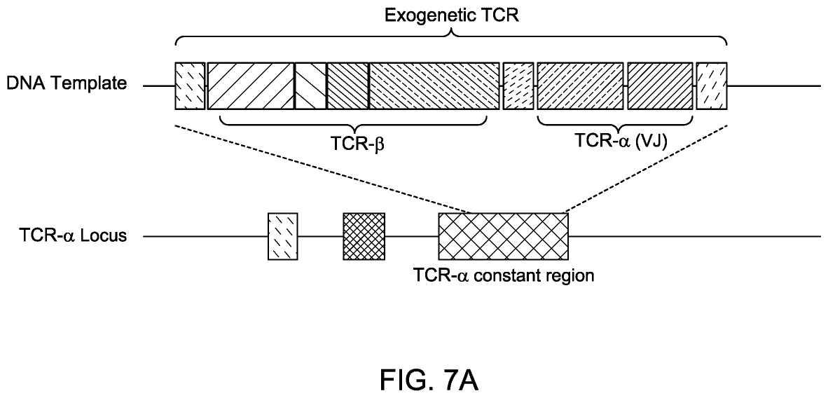

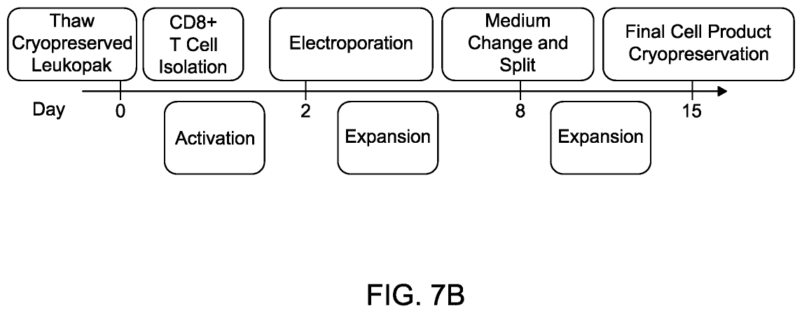

Akt and Erk signaling pathways are activated during the T cell engineering process and their activation correlate with culture growth and viability: To achieve more efficient gene editing and lower toxicity during the TCR engineering process, we co-electroporated human primary CD8+ T cells with CRISPR-Cas9/sgRNA ribonucleoprotein (RNP) and nanoplasmid DNA donor using the Lonza Nucleofector system. DNA plasmid design and targeted insertion location has been previously reported. Briefly, a 1572 bp WT1-peptide-specific TCR template was targeted to TRAC exon 1 (FIG. 7A) utilizing homology-directed repair. Schematics of an optimized 15-day process including T cell activation, electroporation, expansion and harvest is shown in FIG. 7B. To identify signaling pathways that might impact T cell growth and expansion isolated from different donors, T cells isolated from two representative donors were cultured following the same protocol. The Lonza EH115 pulse code was used with the 100 μl cuvette in the Lonza Nucleofector instrument according to the manufacturer's protocol. T cell growth rate and viability were measured during this process, and cell samples were collected at the indicated time points for Western blot analysis.

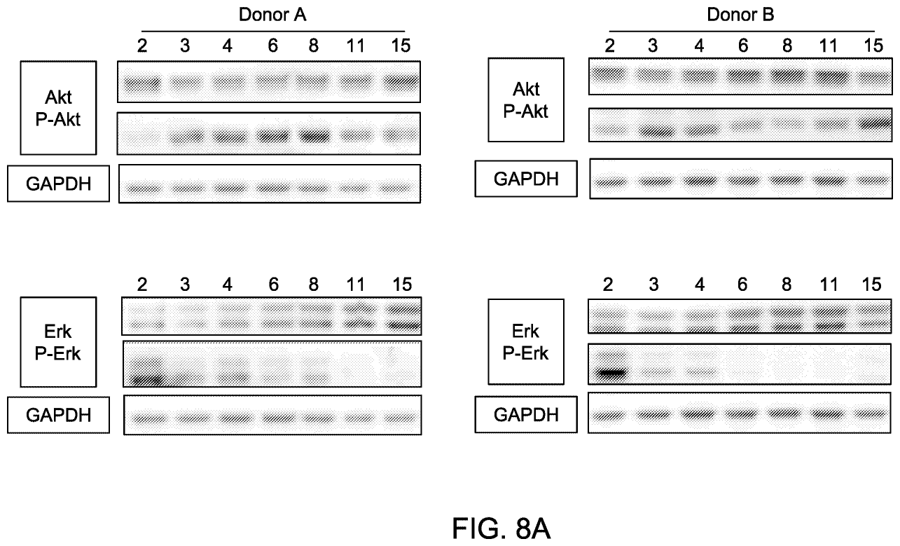

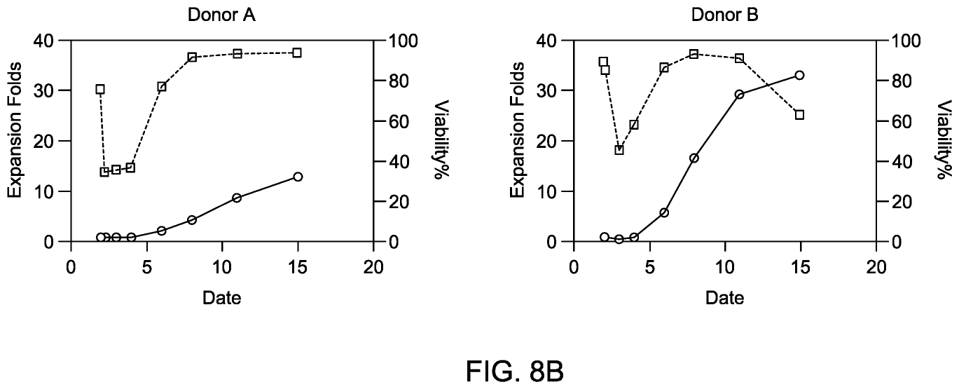

Our data revealed that the lower performing donor cells, donor A, with larger viability loss and lower expansion rates exhibited stronger and more persistent phosphorylation of Akt and Erk compared to the higher performing donor B under the same culture condition (FIGS. 8A and 8B). Akt phosphorylation was detected in donor A on day 3 (24 h post transfection) and remained consistently strong until day 8 and beyond, while Akt phosphorylation in donor B started to taper down on days 4 to 6 (FIG. 8A, top). Similarly, Erk phosphorylation diminished by days 3-4 in the higher performing donor B cells compared to the donor A, in which it lasted up to day 8 (FIG. 8A, bottom. Our findings suggested that activation of Akt and Erk signaling pathways may play a role in T cell growth/expansion post electroporation. Hence, we aim to investigate whether in vitro stimulation of Akt and Erk signaling pathways may enhance growth and expansion rates of the engineered T cells.

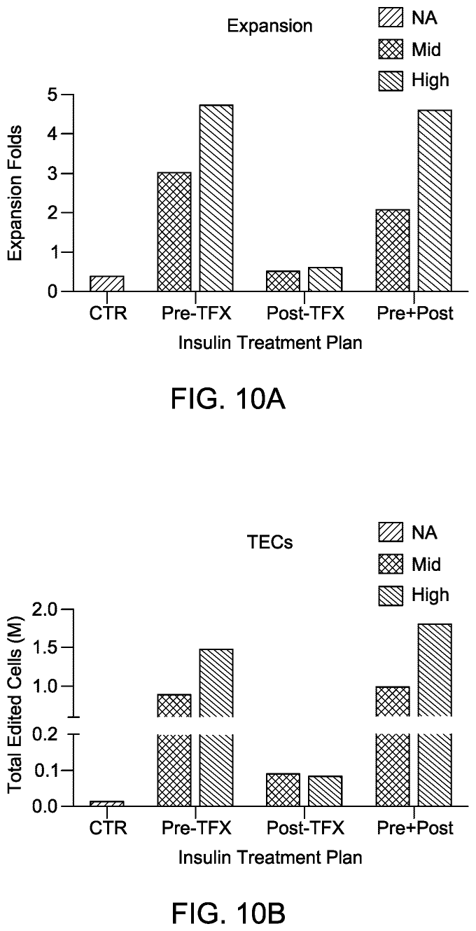

Insulin pre-treatment of cultured T cells prior to electroporation improves culture viability, expansion, and total edited cells during the TCR engineering process: To further investigate the impact of Akt and Erk signaling pathways on T cell growth, T cells obtained from several independent donors were subjected to the T cell engineering process with and without insulin treatment prior to electroporation, as insulin is a known activator of both Akt and Erk signaling pathways. We first tested whether addition of insulin to T cell cultures pre- or post-transfection (pre-TFX or post-TFX), or both pre and post transfection ([pre+post] TFX), has a more pronounced impact on T cell growth. Chemically defined and serum free culture media was used in all experiments as previously reported. While treating cultures with insulin improved T cell expansion under all the tested conditions (pre-, post-, or pre+post TFX treatment), addition of insulin prior to TFX showed the biggest improvement in both cell expansion and TEC levels (FIGS. 10A and 10B). Treating T cell cultures with insulin post-TFX showed a modest effect on cell expansion and TEC levels since cultures treated with insulin either pre-TFX or pre+post TFX had comparable culture growth and TEC levels compared to control (CTR) and post-TFX, respectively.

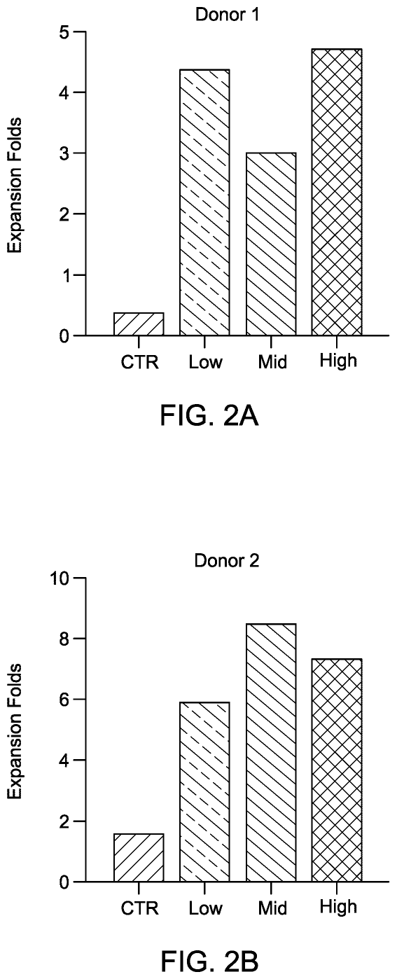

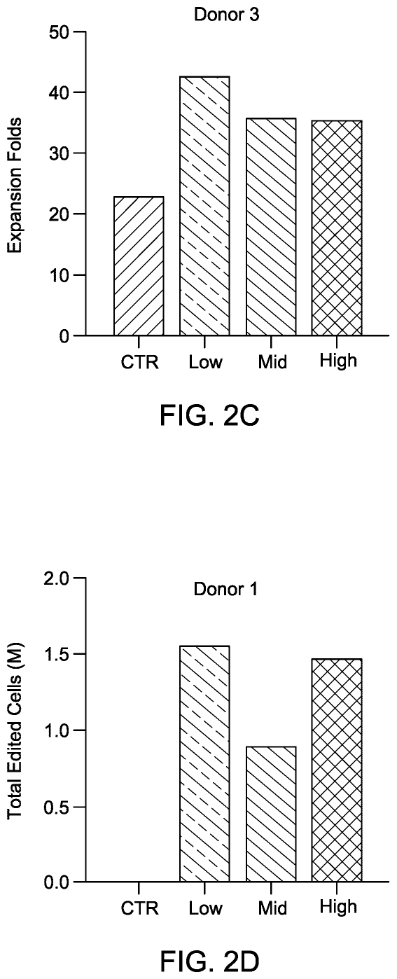

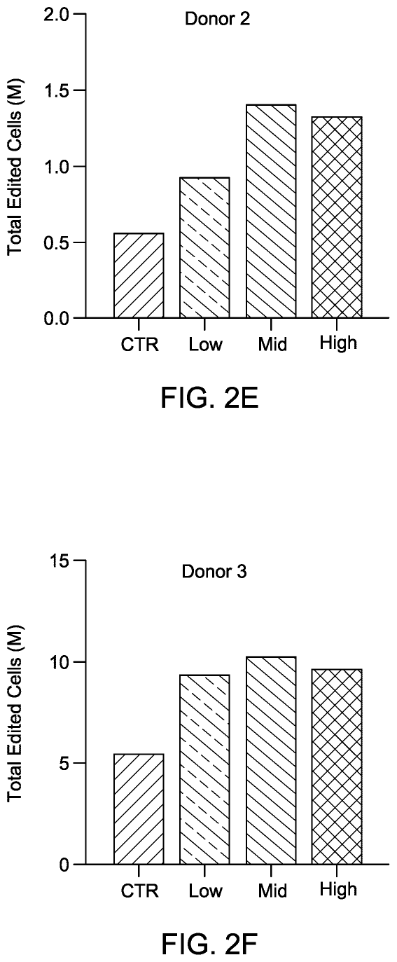

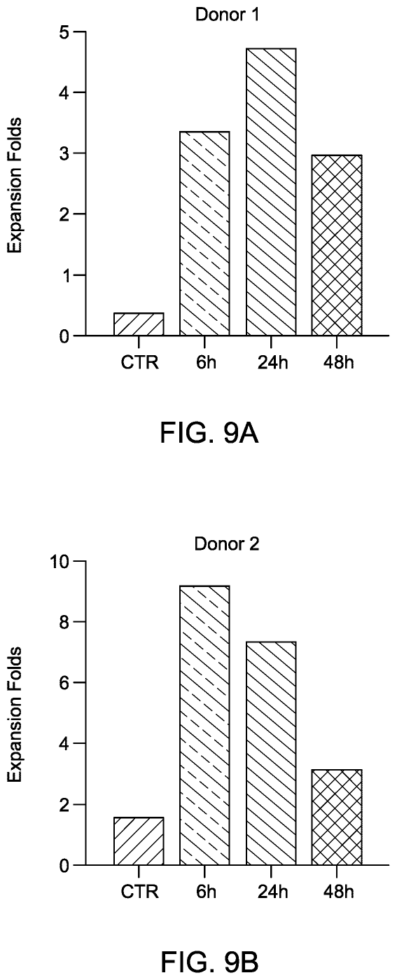

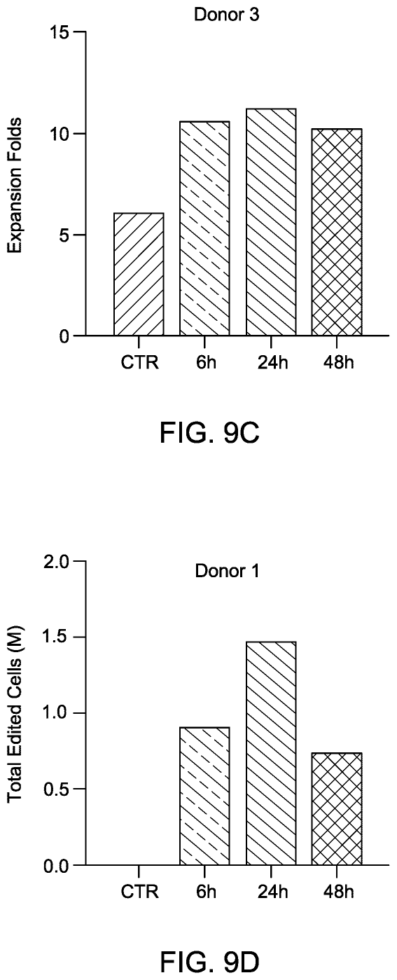

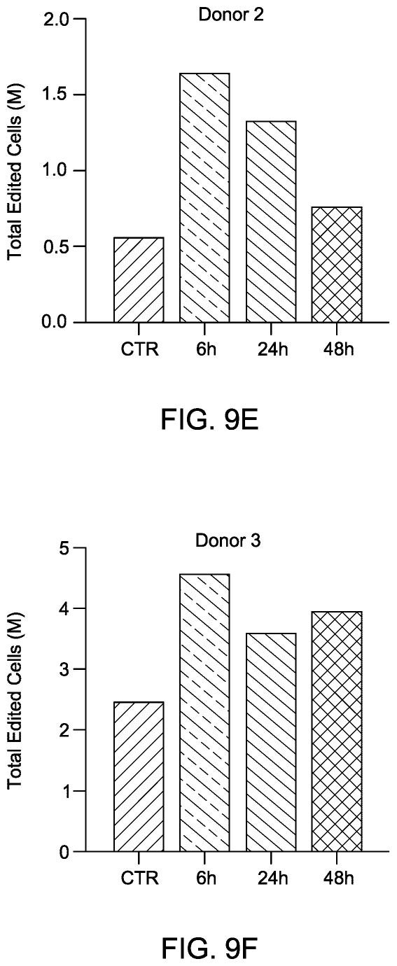

We also tested different insulin pre-TFX concentrations and treatment timepoints and its impact on T cell growth, expansion, and TEC levels. T cells were treated with insulin at the indicated concentrations and only prior to transfection for specific durations. Our data revealed that pre-treatment of T cells 24 h prior to electroporation significantly enhanced T cell expansion (FIGS. 2A-2C) and TEC levels (FIGS. 2D-2F) in a 15-day culture process. Of note, this enhancement was observed in all test concentrations, from low (1 μg/ml) to high (25 μg/ml) concentrations of insulin and across all donors tested. On average, pre-TFX treatment of T cells with insulin resulted in approximately 4-fold improvement in cell culture expansion and TEC levels in all donors. Treating T cell cultures with 25 μg/ml (high) insulin for different lengths of time (6 h, 24 h, 48 h) pre-TFX successfully improved T cell expansion and TEC levels in all donors tested, resulting in an approximately 3-4 folds improvement in both expansion (FIGS. 9A-9C) and TEC levels (FIGS. 9D-9F). Interestingly, shorter insulin pre-TFX treatment lengths (6 h and 24 h) displayed the most enhancement in T cell growth and TEC levels. Thus, insulin treatment at high concentrations and for 6 h and 24 h treatment lengths were used in the ensuing experimental designs.

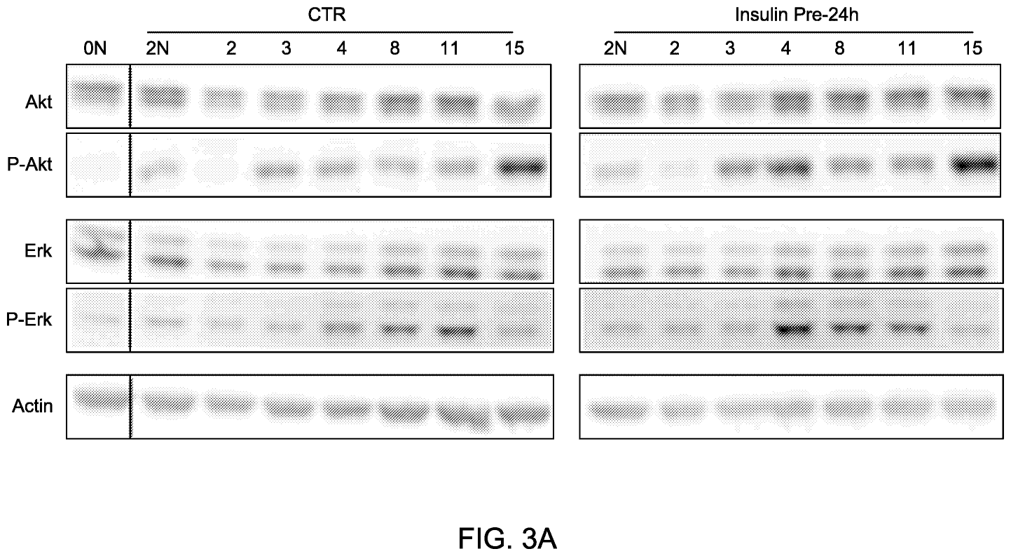

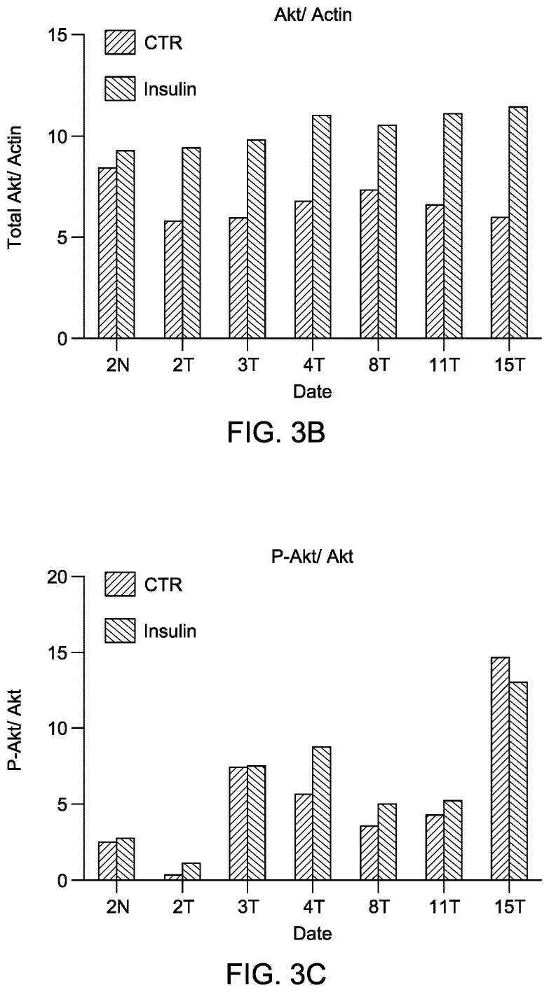

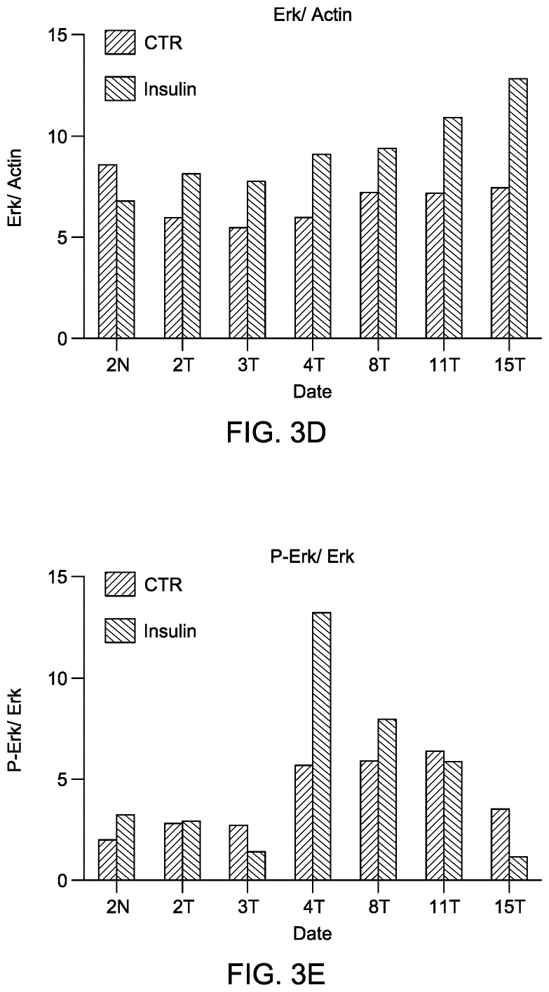

Insulin pre-treatment improves T cell expansion rate and TEC levels by enhancing expression/activation of Akt and Erk signaling pathways: To further investigate the status of Akt and Erk activation during insulin treatment, T cells were treated with insulin (25 μg/ml) 24 h prior to electroporation and cell pellets were isolated from the indicated time points during the T cell engineering process. Western blot analysis of these samples revealed that pre-treatment with insulin increases levels of total Akt and Erk in cultured T cells (FIGS. 3A, 3B, 3D) and although the ratio of phosphorylated Akt (P-Akt)/Akt and phosphorylated Erk (P-Erk)/Erk remained comparable in untreated vs. pre-TFX insulin treated T cells (FIGS. 3C, 3E), the absolute levels of pAkt and pErk were higher in insulin pre-treated T cells (FIG. 3A).

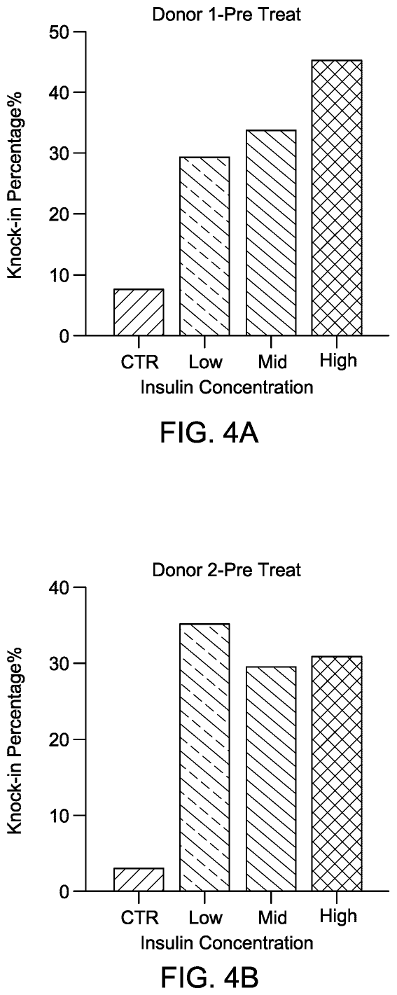

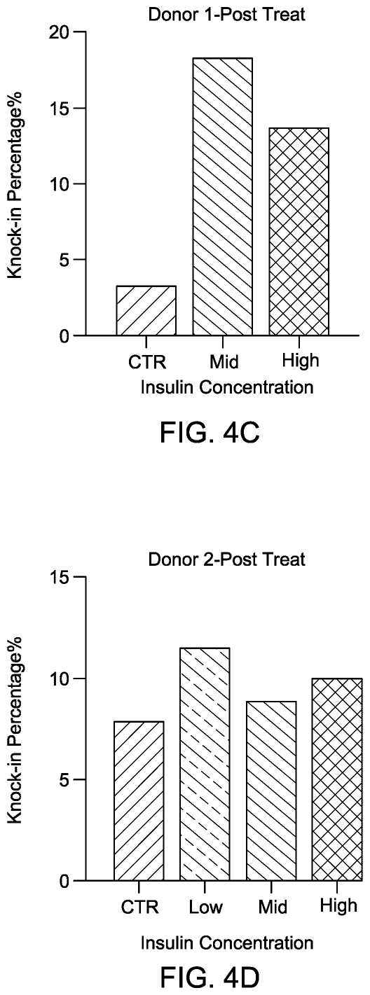

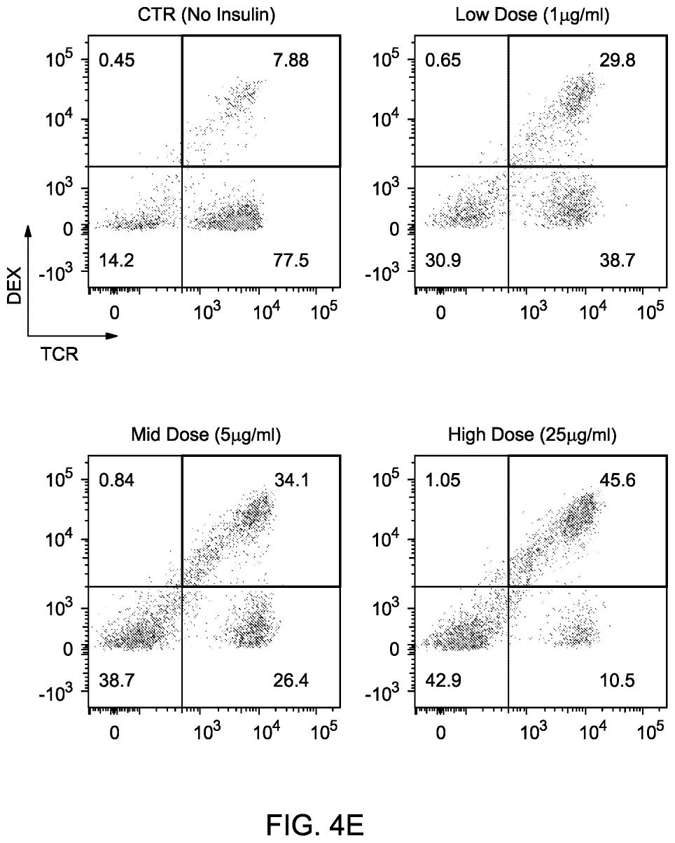

Insulin treatment enhances survival of edited T cells and thus levels of T cell editing in the final drug product: Higher expression/activation levels of Akt and Erk signaling pathways may be the underlying reasons for the observed higher T cell growth during the engineering process. However, T cell growth levels are also impacted by donor-to donor variability and theoretically, for donors with poor cell health conditions, treatment of cultured cells with insulin may improve TCR knock-in rates by improving T cell survival and expansion. Addition of insulin to the cultured T cells should not negatively impact Cas9 mediated cleavage of the genes of interest, nor should it impact the homology-directed repair process in our final T cell product. However, we observed that T cells pre-TFX treated or post-TFX treated with insulin had significantly higher total knock-in percentage than the control group in two independent donors (FIGS. 4A-4D). Pre-TFX insulin treated T cells reached 35.5% (low insulin concentration), 29.8% (mid insulin concentration) and 31.3% (high insulin concentration) knock-in efficiency compared to the control group with only 3.25% KI efficiency in donor 1 (FIG. 4A). Consistent with this trend, pre-TFX insulin treated T cells also reached 29.8% (low), 34.1% (mid) and 45.6% (high) knock-in efficiency, which was significantly higher compared to the control group (7.88% KI) in donor 2 (FIG. 4B). T cells treated with insulin post-TFX exhibited 18.4% (mid) and 13.8% (high) knock-in efficiencies in donor 1 and 11.6% (low), 8.98% (mid) and 10.1% (high) in donor 2.

Although in post-TFX conditions the KI improvement was not as robust as in the pre-TFX conditions, insulin treatment still exhibited higher TCR editing values compared to the control group (FIGS. 4C and 4D). The observed improvements in knock-in ratio in the final T cell product is likely due to the survival and growth support of the edited T cells. The observed improvements in TEC levels trend with higher expansion folds in T cell cultures pretreated with insulin. Without being bound by theory, the observed increase in editing efficiency in the FDP (FIG. 4E) may be due to improved metabolic support in the treated cells.

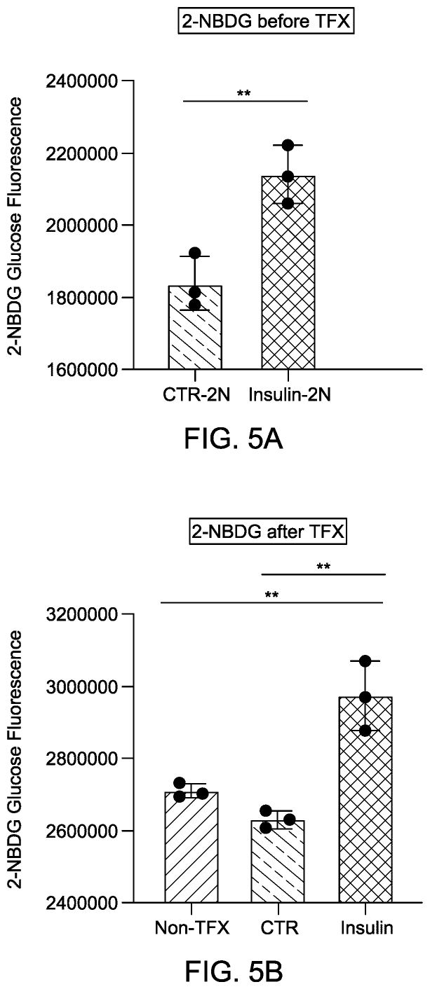

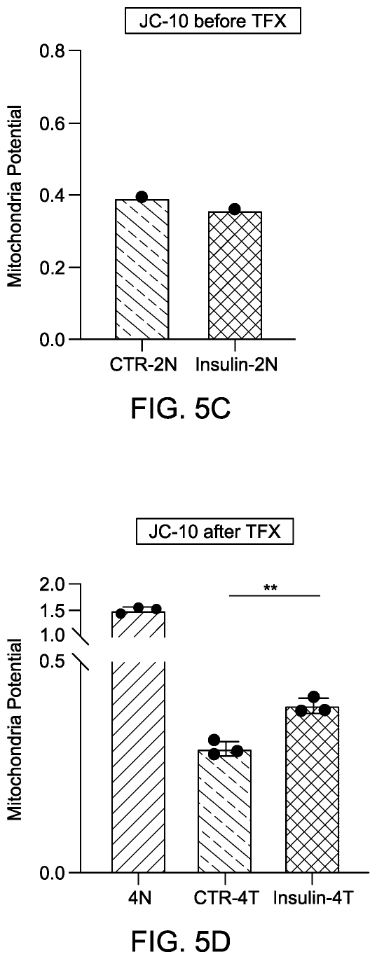

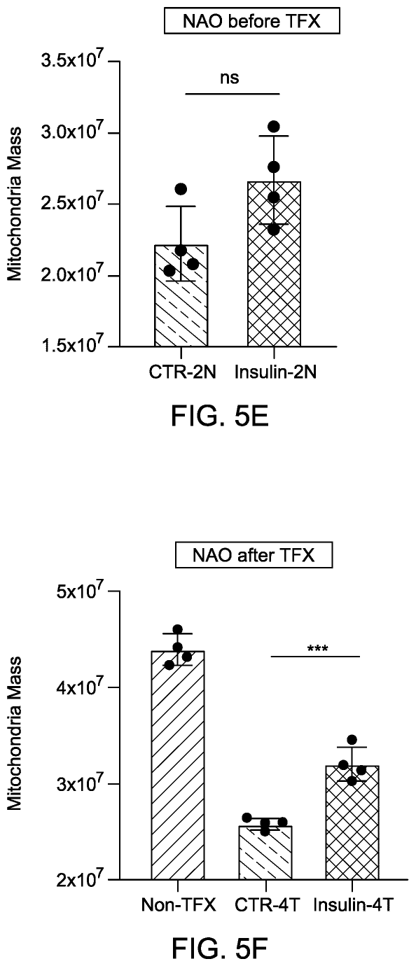

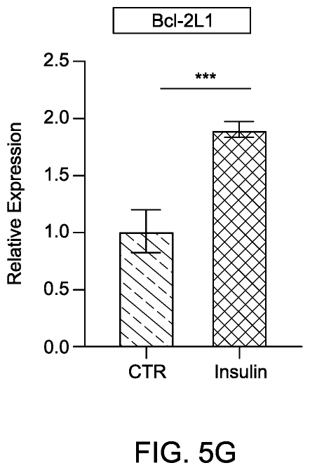

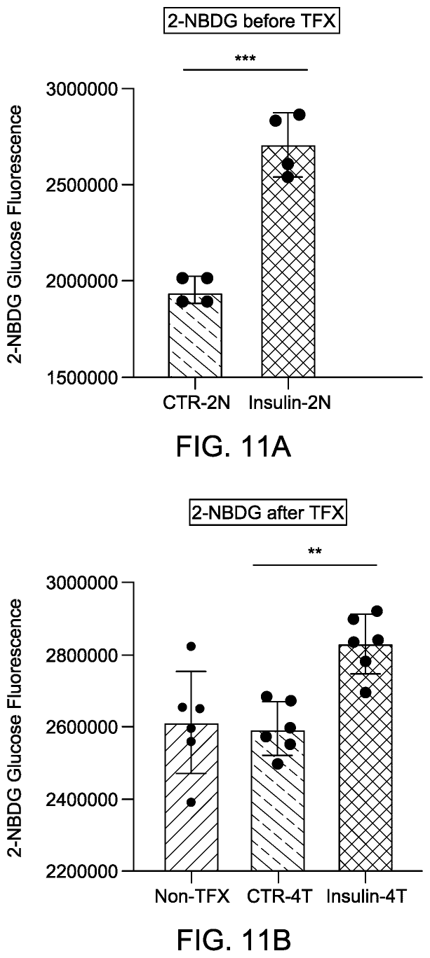

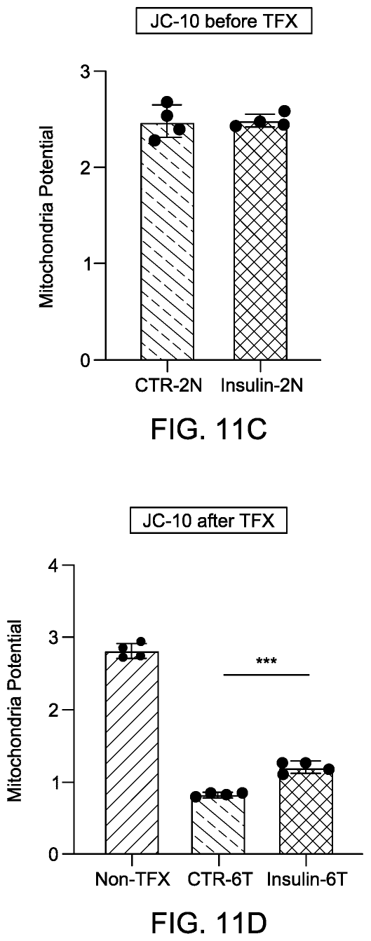

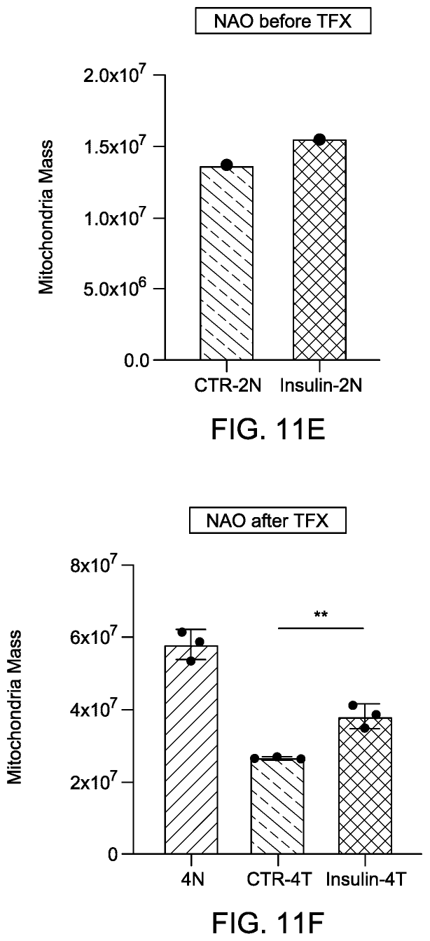

Insulin pre-treatment enhances T cell metabolism and mitochondrial function: To further understand how insulin pre-treatment might enhance T cell growth and TCR editing efficiency, we investigated the impact of insulin pre-treatment on T cell metabolism and mitochondrial function. A cell-permeable fluorescent glucose analogue, 2-NBDG, was used to measure glucose uptake in T cells. 2-NBDG has been widely used for evaluating cell metabolism and proliferation. T cells were treated with 25 μg/mL of insulin for 24 h. The rate of 2-NBDG uptake was measured in untreated (control) or insulin pretreated T cells prior to transfection (FIG. 5A). T cells pretreated with insulin displayed significantly higher rates of 2-NBDG uptake and accumulation compared to the control group (FIG. 5A). The same trend was maintained 48 h post transfection (FIG. 5B) even though insulin was washed away thoroughly prior to transfection by electroporation, which is indicative of a much higher T cell metabolism rate both before and after the transfection process. Of note, insulin treated T cells had significantly higher metabolism rates compared to both untreated as well as un-transfected/untreated control groups (FIG. 5B). This implies that the higher rates of T cell metabolism bestowed by insulin treatment is maintained during and post electroporation process in a donor independent manner as similar trends were observed for different independent donors (FIGS. 5A-5B, FIGS. 11A and 11B).

We also investigated the potential reasons for enhanced metabolism upon insulin pre-treatment. Since mitochondria function and integrity can be compromised during electroporation process, which may also trigger cell apoptosis, we tested the effect of insulin pre-treatment on mitochondria function. T cells were treated with JC-10 to measure mitochondrial membrane potential (MMP) levels during T cell engineering process. While prior to transfection we observed comparable MMP levels between insulin pretreated and untreated control groups (FIGS. 5C and 11C), insulin pretreated T cells showed statistically significant higher MMP levels when tested 48 h post electroporation (FIGS. 5D and 11D) in a donor independent manner. Additionally, our data confirmed that the process of electroporation can negatively impact MMP levels in T cells even 48 hours post transfection, when compared to untransfected control group (FIGS. 5D and 11D).

Mitochondria mass is another indicator of mitochondria health in cells, and electroporation-mediated mitochondrial damage can result in loss of mitochondria mass and attenuated mitochondrial function. We used Nonyl acridine orange (NAO) to measure the mitochondria mass in insulin pre-treated or untreated T cells and observed a significant and donor-independent mitochondrial mass reduction in untreated T cells, compared to insulin pre-treated T cells, post electroporation (FIGS. 5F and 11F). Note that prior to electroporation both insulin pretreated and untreated T cells have comparable mitochondria mass (FIGS. 5E and 11E), suggesting that mitochondria mass loss is mainly due to electroporation process.

We further looked at mRNA transcription levels of a few members of Bcl-2 family of proteins, which can enhance mitochondrial membrane integrity by blocking Bax/Bak mediated mitochondrial membrane permeabilization and hence attenuating apoptosis. The qPCR data showed considerably higher levels of Bcl-2L1 mRNA in insulin pretreated (24 h) T cells (FIG. 5G). Bcl-2L1 is one of the crucial markers that is reported to play an important role in maintaining mitochondria health and function. Higher levels of Bcl-2L1 expression in insulin pretreated T cells may effectively offset the negative impact of mitochondrial membrane permeabilization during electroporation.

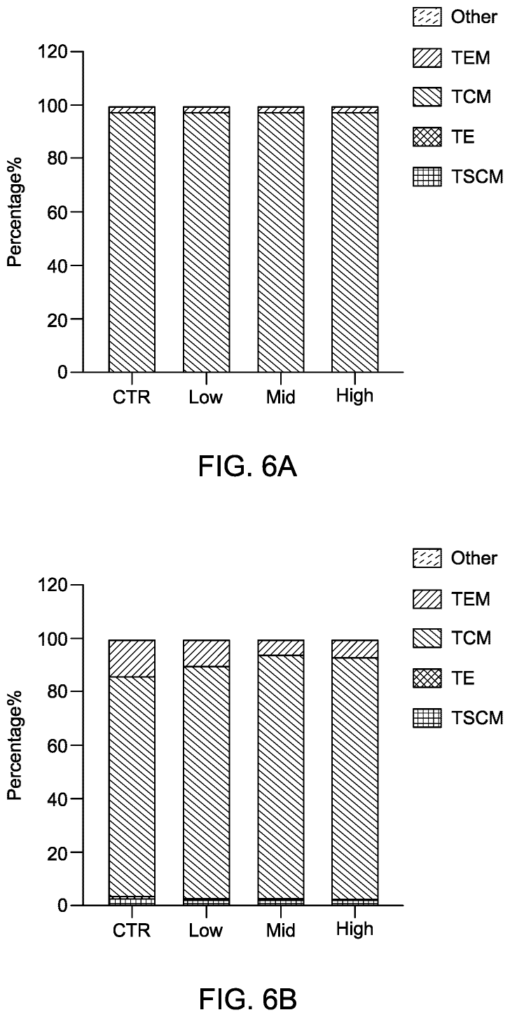

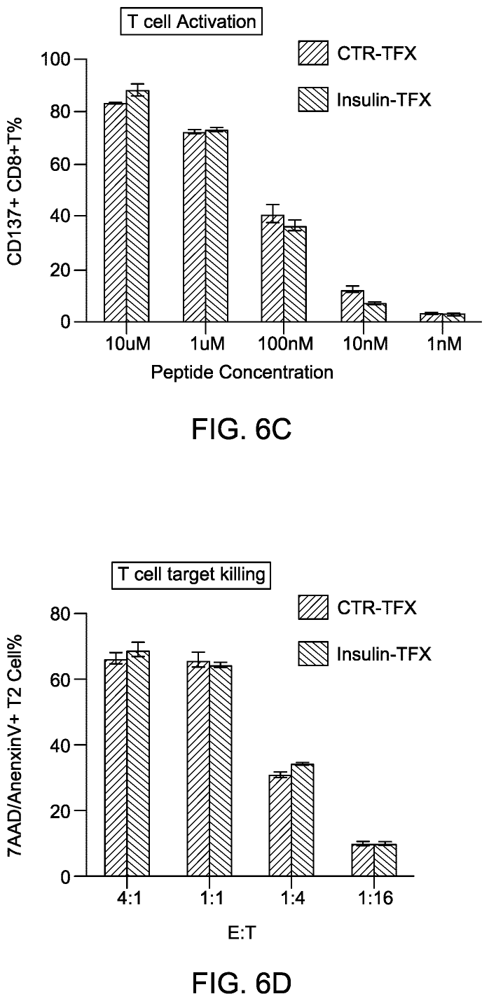

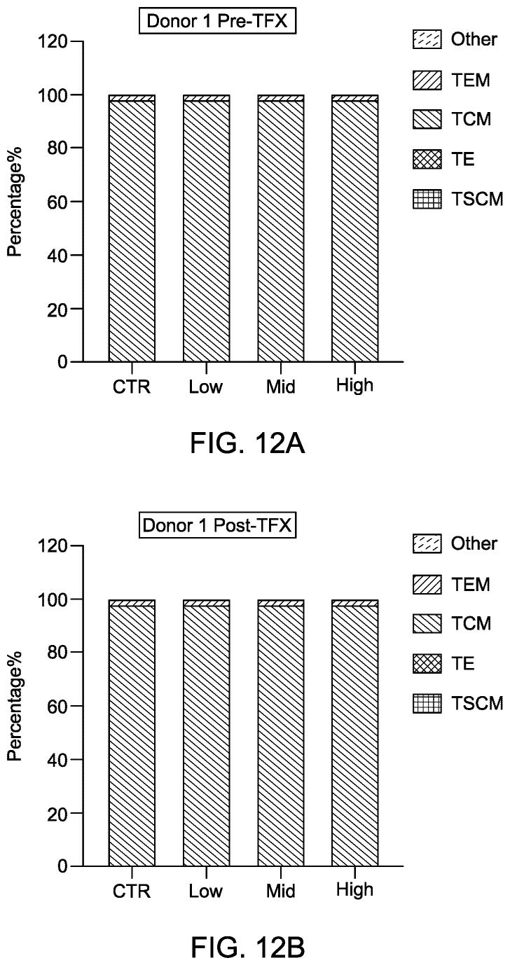

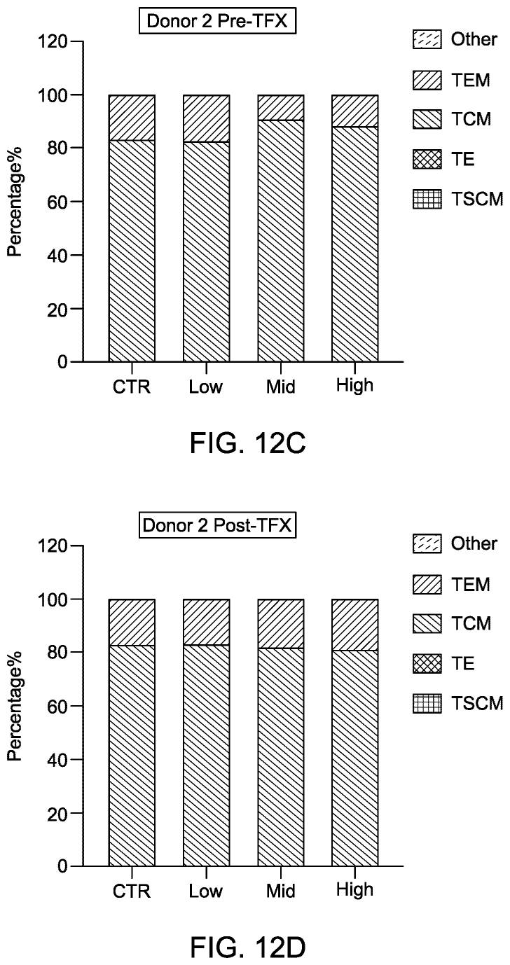

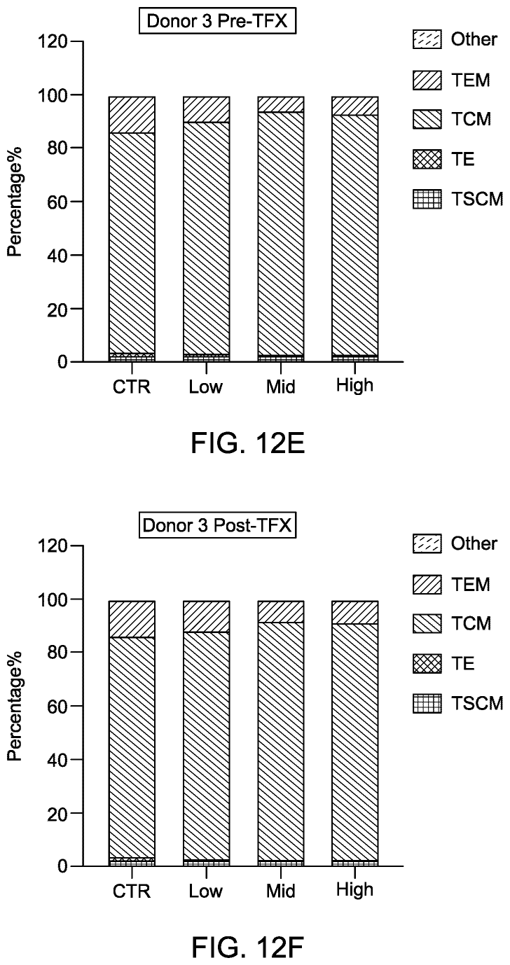

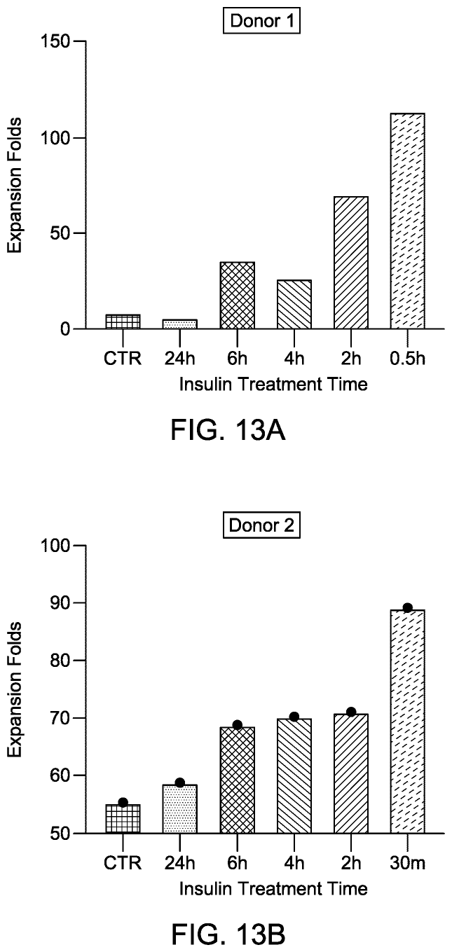

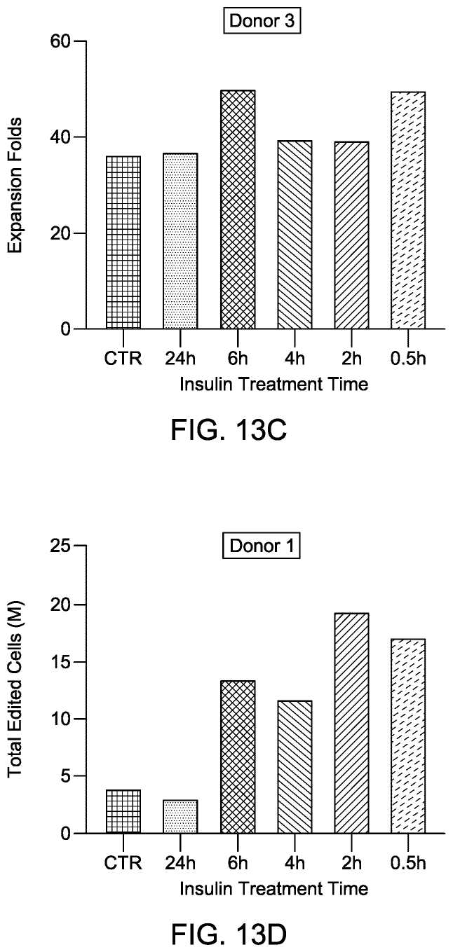

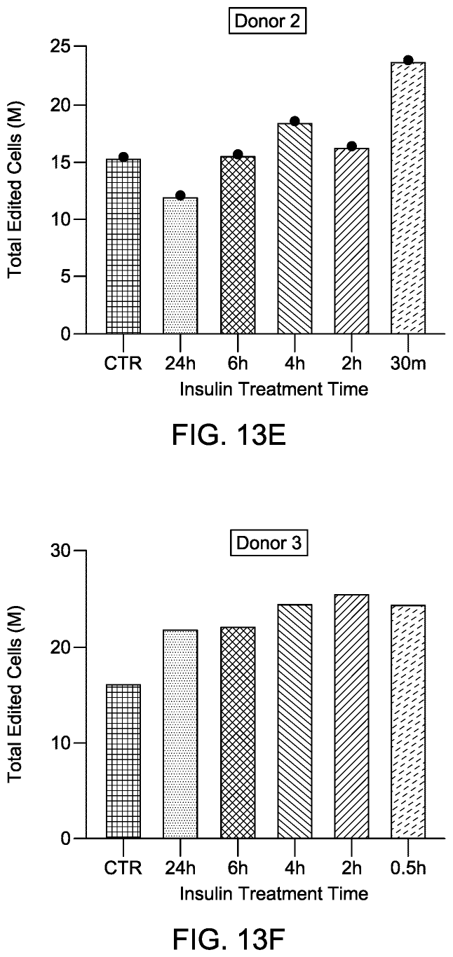

Insulin pre-treatment does not impact FDP characteristics and T cell function: Higher levels of T cell memory phenotype in the FDP strongly correlates with clinical efficacy during T cell therapy. Memory T cells, including central memory T cells (TCM) and stem cell memory T cells (TSCM) specifically, have the potential for proliferation and long-term survival once reintroduced back to the patient. Our T cell engineering protocol yielded upwards of 90% TCM and TSCM in the FDP (FIGS. 6A-6B, FIGS. 12A-12B). The high percentage of memory T cell phenotype may promote higher rates of proliferation and enhance tumor killing ability of the T cells. Pre-TFX or post-TFX insulin treatment of T cells, tested in different donors, did not impact FDP T cell phenotype compared to the untreated control groups, (FIGS. 6A-6B, FIGS. 12A-12F). Insulin pre-treatment of T cells resulted in a significant improvement in cell expansion rates, cell editing efficiency, and hence total edited cell numbers in the engineered T cells.

To test the impact of such changes on T cell function, CD8+ T cells derived from insulin pretreated or untreated conditions were compared in T cell activation and target cell killing assays (FIGS. 6C-6D). FDP from several donors were cryo-preserved and then thawed prior to use. Cells from either untreated control (CTR) or insulin pre-treated groups were cultured for 24 h with the indicated concentrations of a specific peptide (WT1), which binds to the engineered TCR. Flow cytometry analysis revealed comparable CD137, a T cell activation marker, expression profile between T cells derived from insulin pretreated or untreated conditions (FIG. 6C), suggesting that insulin treatment did not affect T cell activation.

T cell functionality was tested using a T cell mediated target cell killing assay. Target cells T2 (T) were labeled with CFSE-far red, and then pulsed with 20 μM WT1 peptide before co-incubation with T cell FDP (E) at indicated E:T ratios. These data confirmed that T cells pretreated with insulin had comparable target cell killing ability to that of untreated control groups at all different E:T ratios (FIG. 6D), suggesting that insulin treatment does not impact antigen-specific tumor cell killing by the engineered T cells.

Taken together, our data demonstrate the safety of using insulin pre-treatment during the T cell engineering process, outlining the broad impact of growth factor stimulation in T adoptive cell transfer field, specifically in improving T cell engineering efficiency, increasing expansion rates, and increasing total edited cell numbers. By improving T cell growth and editing efficiency post-electroporation, T cells with three- or four-fold higher TECs with comparable activation, proliferation, and killing ability to the untreated control groups can be obtained. Of note, impact of insulin pre-treatment is not limited to donors or restricted by antigens, and all the tested independent donors showed a consistent trend of growth enhancement and editing improvement upon insulin pre-treatment.

Materials and Methods: Ethics statement: All experimental methods were carried out in accordance with the approved guidelines. All donor material was purchased from commercial vendors with signed informed consent forms stored at the vendor site. All cell culture procedures and processes have been documented in accordance with guidelines approved by Genentech, Inc.

RNA ribonucleoprotein and DNA plasmid: Single guide RNA sequences for both TRAC and TRBC were derived as described by Oh, Senger et al and ordered from Synthego (Menlo Park, CA, USA). SpyFi Cas9 protein was purchased from Aldevron (Fargo, ND, USA) and preformed RNPs (60 pmol total), HDR template (<8 μg), and T cells resuspended in P3 buffer as we published before. Nanoplasmid with WT1 TCR sequence was ordered from Nature Technologies (Lincoln, NE, USA) and was used at a working concentration 150 μg/ml in electroporation.

Cell isolation and activation: Peripheral blood mononuclear cells (PBMCs) were isolated from human cryopreserved leukopak using Ficoll gradient centrifugation (400 rpm, 25 min). All samples were obtained from healthy donors with HLA A*02:01 MHC class I complex. CD8+ T cells were isolated using the Miltenyi AutoMACS cell separator according to the manufacturer's protocol. Isolated CD8+ T cells were then mixed with Miltenyi T cell TransACT reagent (1:100) (Catalog #130-111-160), 25 ng/mL IL-7 (Catalog #130-095-367), and 50 ng/mL IL-15 (Catalog #130-095-760) in FUJI Prime-XV medium (IrvineScientific Catalog #91154) for 48 h for activation.

Electroporation: Cells were electroporated with the 4D-Nucleofector System (Lonza) according to the manufacturer's protocol. After activation and insulin treatment, 1×10⁷ (10 million) CD8+ T cells were washed and resuspended with Lonza P3 primary cell nucleofector solution (Catalog #V4XP-3024) and then mixed with pre-mixed RNP and DNA plasmid before transferring to Lonza 100-μL cuvette. After electroporation, 400 μl of FUJI Prime-XV medium was added to the cuvette and incubated for 15 min before seeding cells in culture flask. The pulse code used in this manuscript is EH115 for all experiments.

Cell Culture: Insulin was made in house (GNE). Unless stated otherwise, cells were cultured with FUJIPrime-XV medium with 25 ng/mL IL-7 and 50 ng/mL IL-15 (Complete medium). After electroporation, T cells were carefully transferred to 24-well gREX (Wilson Wolf, Catalog: 80192M) at 1-2×10⁶ cells/cm² seeding density. 8 mL of complete medium were added per well and 50% of the medium was exchanged 6 days later. Cells were incubated at 37° C. and 5% CO₂. The NucleoCounter NC-200 automated cell counter with the Via2-Casette were used in this study for cell counting and viability analysis.

Flow Cytometry: All surface and intracellular staining antibodies were purchased from Biolegend and BD Biosciences as listed in Table 1. Dextramer antibodies for knock-in analysis were purchased from Immudex (Catalog: WB3469-PE, WT1) and used following manufacturer's protocol. Surface staining, CFSE staining, 7-AAD/annexin V staining, and intracellular staining were performed as previously described. Samples were analyzed using the BD FACSLyric flow cytometer (BD Biosciences) and FlowJo v10 software. For proliferation and cell killing assay, data were corrected by a negative control or target cell-only control.

Western Blot: Antibodies for Western blot were purchased from Cell Signaling and listed in Table 2. T cell pellets were collected, washed with PBS and stored as frozen pellets in −80° C. until use. Cell pellet processing, Western blot experiments and data analysis were performed as previously described (Tang, J B C, 2020).

gPCR: RNeasy Mini Kit (Cat. #74106) was purchased from Qiagen. TaqMan RNA-to-CT 1-Step Kit (Cat. #4392938) and Taqman primer-probe assays with reporter dye FAM and MGB quenchers were purchased from Thermo Fisher (assay information listed in Table 3). After RNA isolation, 2 ng RNA was mixed with Taqman primer-probe mix, TaqMan RNA-to-CT 1-Step master mix, and RT enzyme mix (10 μL total volume) according to the manufacturer's protocol. Reactions were measured using QuantStudio 6 Flex machine and data was analyzed using QuantStudio Real-Time PCR software v1.2. PCR cycling conditions were 50° C. for 30 min, 95° C. for 10 min, 40× cycles of 95° C. for 15 s, 60° C. for 1 min. All data points were collected in triplicates, using RNA18S and GAPDH as internal control.

Glucose Uptake Analysis: Glucose uptake assay was performed on insulin pre-treated CD8+ T cells or non-treated control cells according to the manufacturer's protocol (Selldeckcome, catlog. S8914). Briefly, human CD8+T cells were collected and plated in a 96-well flat bottom plate at 50,000 cells/well and then incubated in an incubator with glucose-free medium for 4 h. Then T cells were stained with 2-(N-(7-Nitrobenz-2-oxa-1,3-diazol-4-yl)Amino)-2-Deoxyglucose (2-NBDG, Selldeckcome, catalog. S8914) in glucose-free RPMI1640 for 30 min at 37° C. Excess 2-NBDG was washed away twice with PBS. Finally, 2-NBDG uptake was measured by SpectraMax Paradigm Multi-Mode Microplate Reader at 525 nm (F1).

JC-10 Mitochondrial Membrane Potential Analysis: Mitochondrial membrane potential was assessed according to the manufacturer's protocol (Abcam. #ab 112134, Abcam). Briefly, human CD8+T cells were collected and plated in a 96-well flat bottom plate at 50,000 cells/well and JC-10 dye-loading solution was added after cells were washed once with PBS. Cells were incubated in an incubator (37° C., 5% CO₂) for 30 min after which SpectraMax Paradigm Multi-Mode Microplate Reader at 525 nm (F1) and 585 nm (F2) was used to measure the readouts. Ratio of F2/F1 is the JC-10 relative expression ratio.

Nonyl acridine orange (NAO) mitochondria mass analysis: Mitochondrial mass analysis was assessed according to the manufacturer's protocol (Life Technologies catalog No. A1372). Briefly, human CD8+T cells were collected and plated in a 96-well flat bottom plate at 50,000 cells/well and then Nonyl acridine orange dye-loading solution (2.5 μM) was added after cells were washed once with PBS. Cells were then incubated in an incubator (37° C., 5% C02) for 30 min after which the SpectraMax Paradigm Multi-Mode Microplate Reader at 525 nm (F1) was used to measure readouts.

Data Analysis and Statistics: Western blot data were analyzed with Image Lab 6.1 (Bio-Rad). Flow cytometry data were analyzed using FlowJo software v10 (BD Biosciences). Graphs were created and presented with Graphpad Prism 10. For all statistical analysis, data has been presented as mean±SEM.

TABLE 1
Flow Cytometry Antibody List

| Antibody Target | Fluorescence | Vendor Name | Catalog Number |
|---|---|---|---|
| CD3 | BV510 | BD Biosciences | 563109 |
| CD3 | APC | BD Biosciences | 340440 |
| CD8 | APC-Cy7 | BioLegend | 300926 |
| CD27 | PerCP-Cy5.5 | BioLegend | 302820 |
| CD45RO | BV421 | BioLegend | 304224 |
| CD45RA | PE | BioLegend | 304108 |
| CD62L | BV605 | BD Biosciences | 562720 |
| CD95 | PE-Cy7 | BD Biosciences | 561633 |
| CD137 | PE | BioLegend | 309804 |
| CD197 | AF700 | BD Biosciences | 561143 |
| 7-AAD | NA | BioLegend | 79993 |
| Annexin V | FITC | BioLegend | 640906 |
| DAPI | NA | Miltenyi Biotec | 130-111-570 |
| TCR-αβ | Percp-Cy5.5 | BioLegend | 306724 |

TABLE 2
Western Blot Antibody List

| Antibody Target | Vendor Name | Catalog Number |
|---|---|---|
| Akt | Cell Signaling | 4619s |
| p-Akt | Cell Signaling | 4060s |
| Erk | Cell Signaling | 4695s |
| p-Erk | Cell Signaling | 4370s |
| STAT5 | Cell Signaling | 94205s |
| p-STAT5 | Cell Signaling | 9359s |
| Actin-HRP | Cell Signaling | 5125s |
| GAPDH | Cell Signaling | 2118s |
| GAPDH-HRP | Cell Signaling | 3683s |

TABLE 3
qPCR Primer List

| | | Thermo Fisher |
|---|---|---|
| qPCR Target | Assay ID | Catalog Number |
| Bc1-2L1 | Hs00236329_m1 | 4331182 |
| GAPDH | Hs02758991_g1 | 4331182 |
| RNA18S | Hs03928990_g1 | 4331182 |

### Example 2: Materials and Methods for Clinical Scale Manufacturing

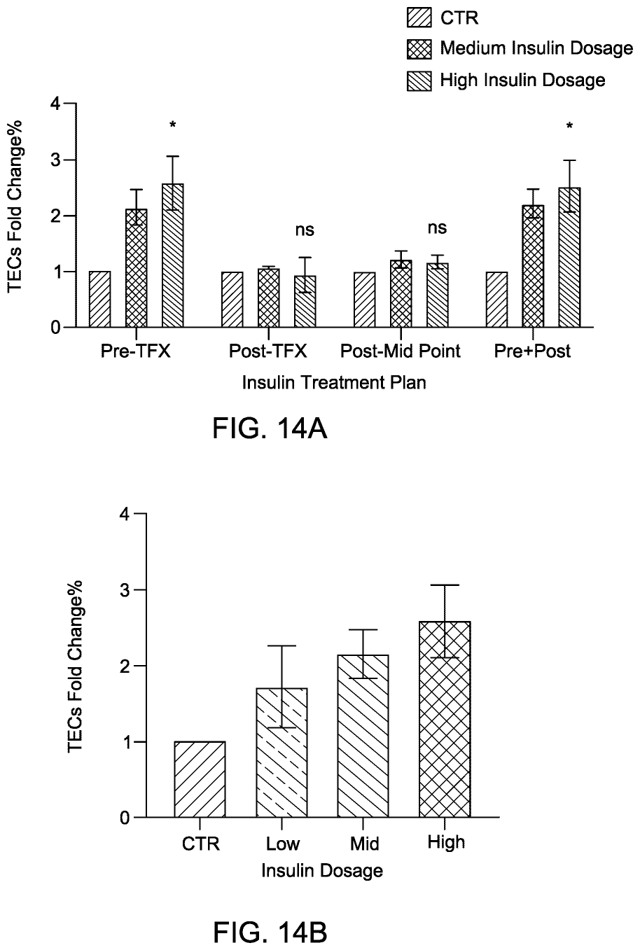

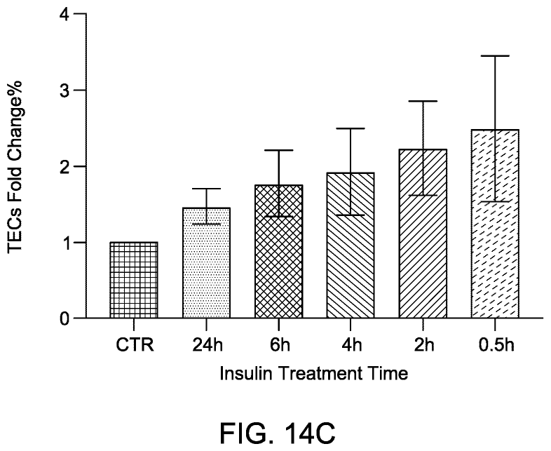

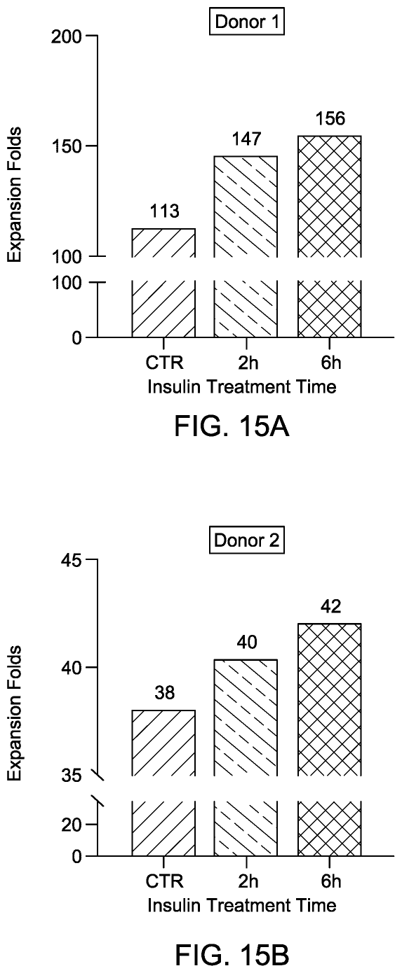

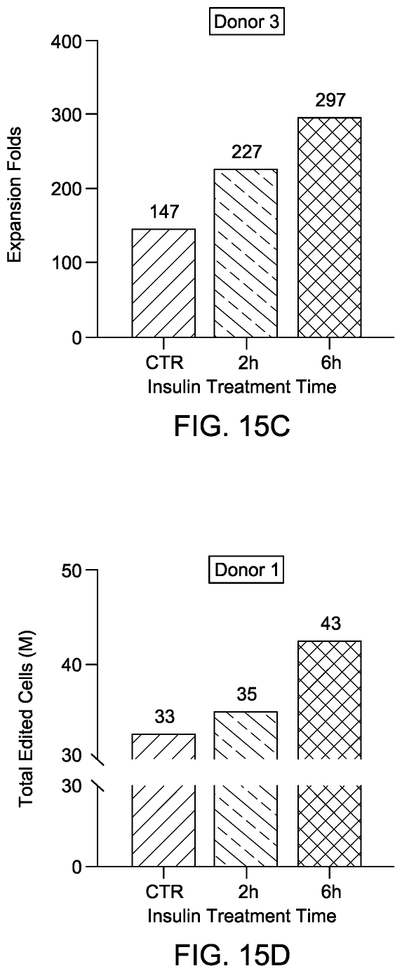

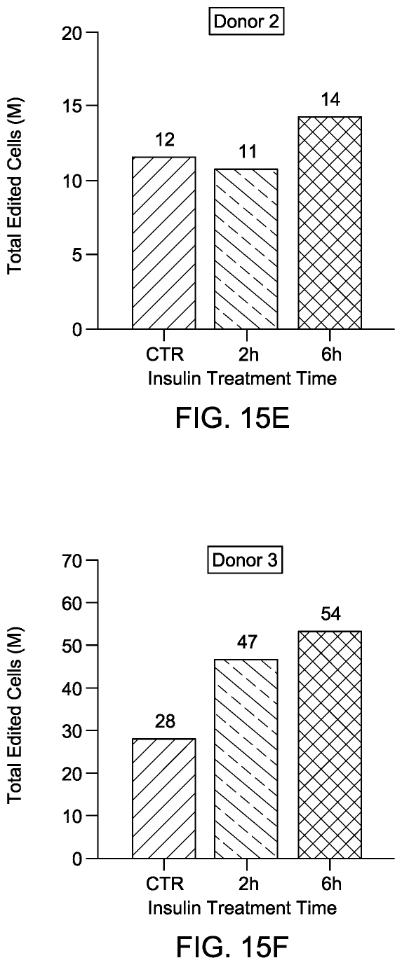

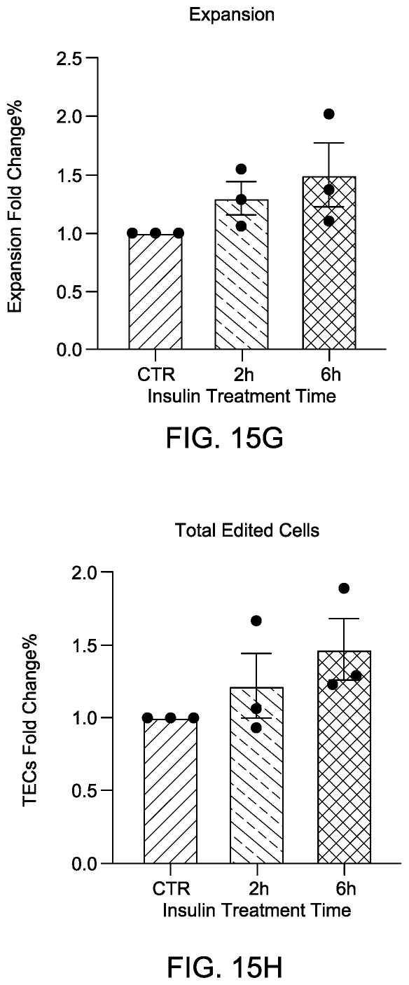

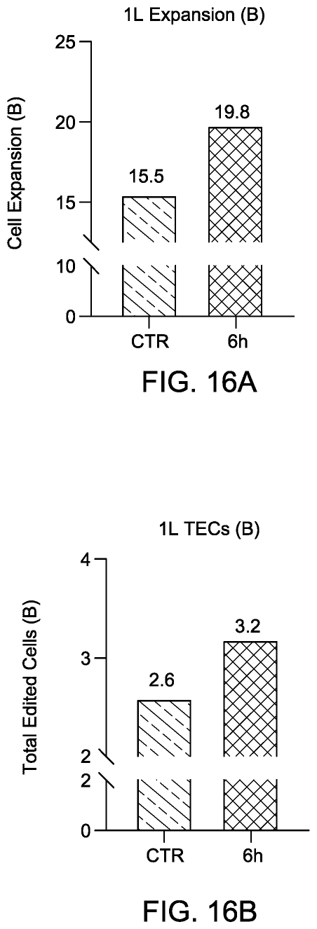

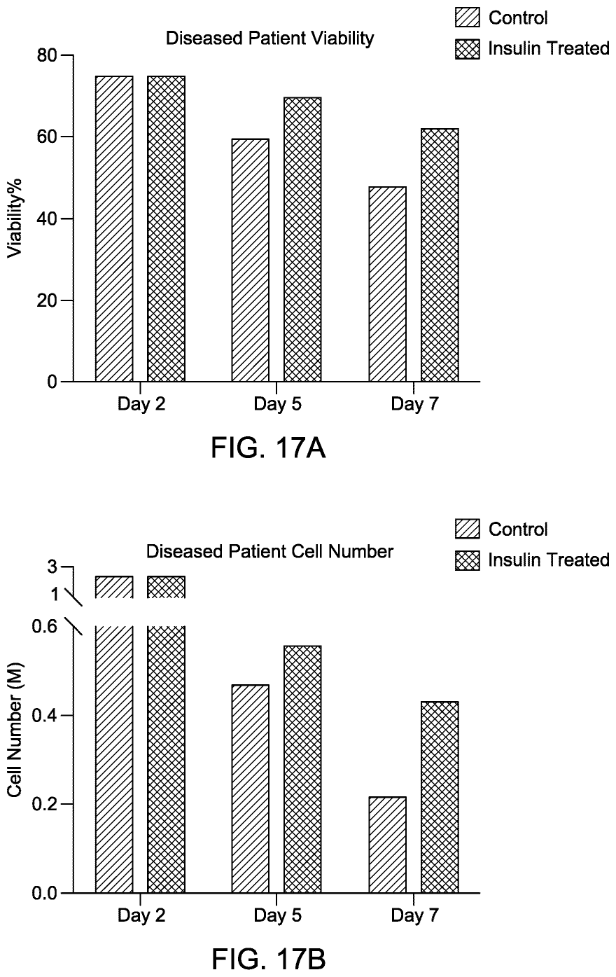

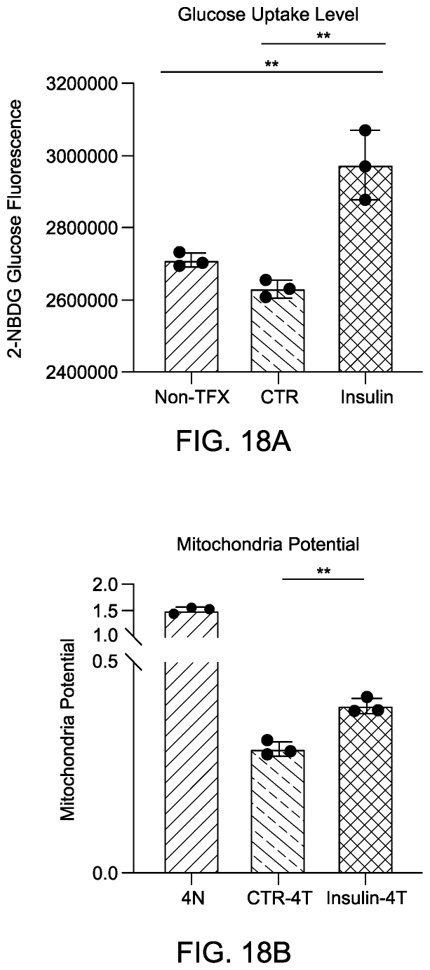

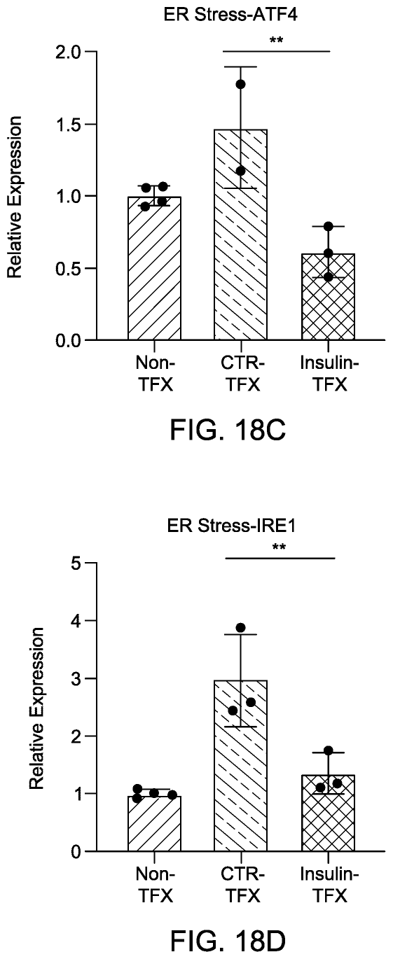

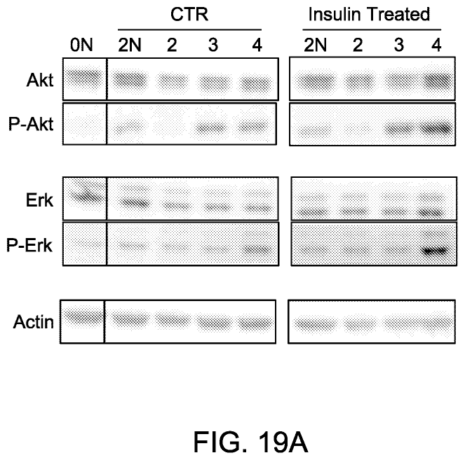

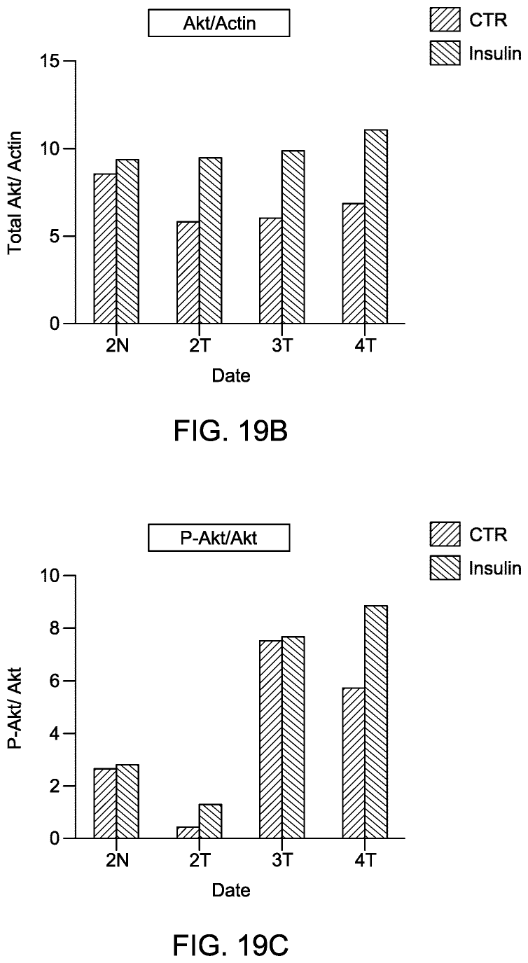

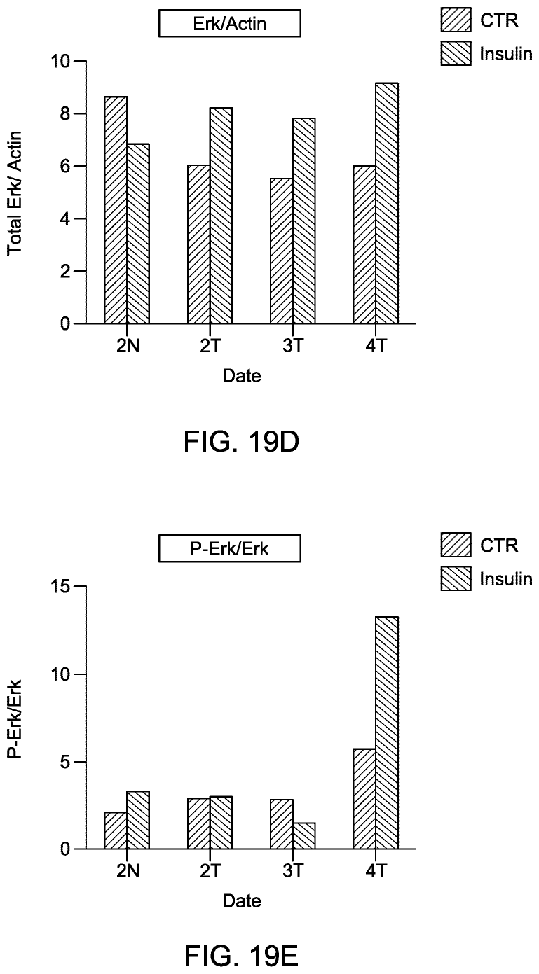

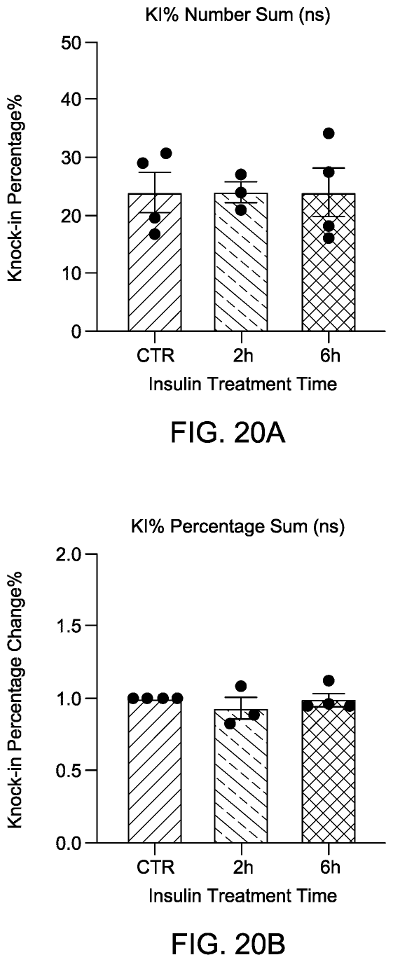

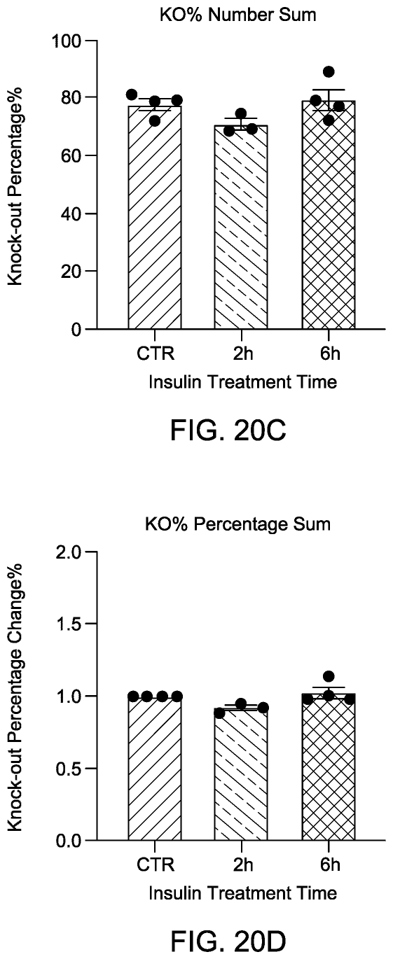

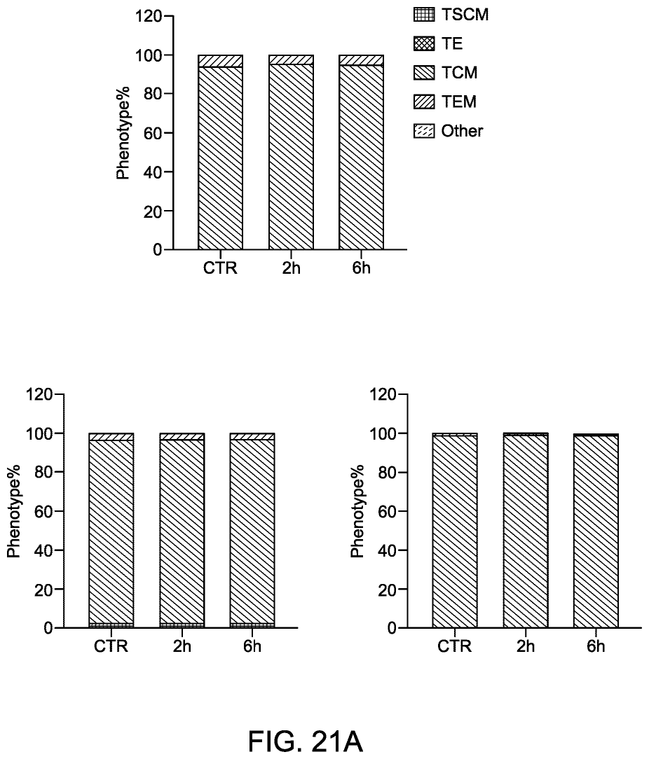

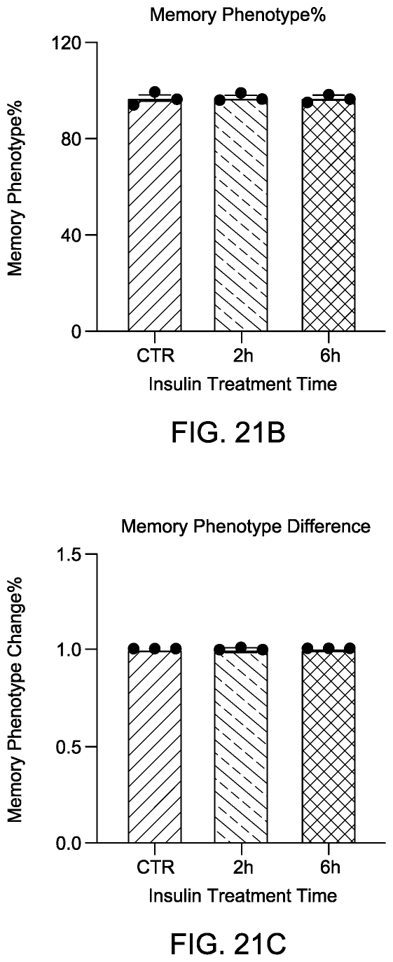

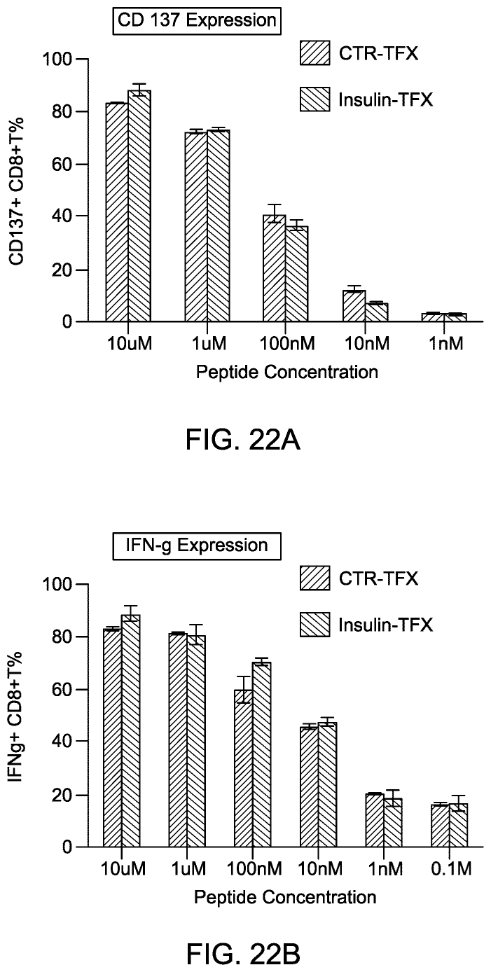

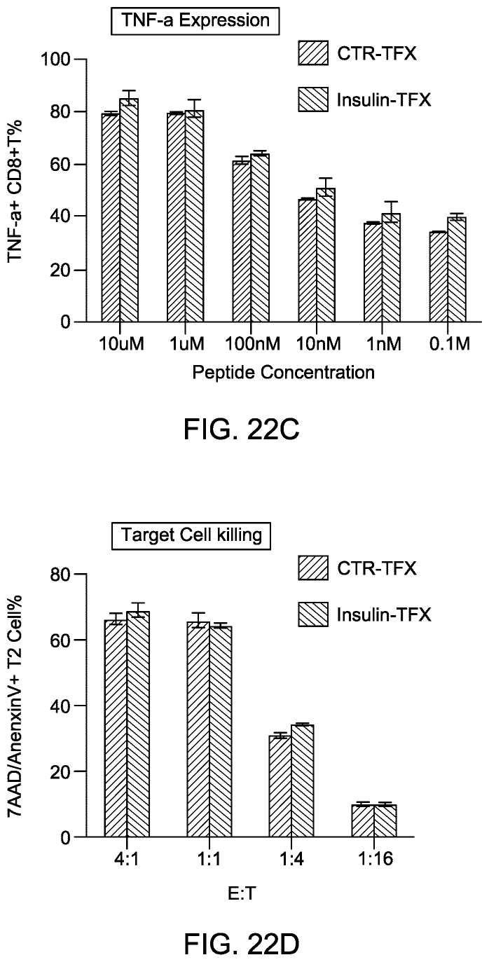

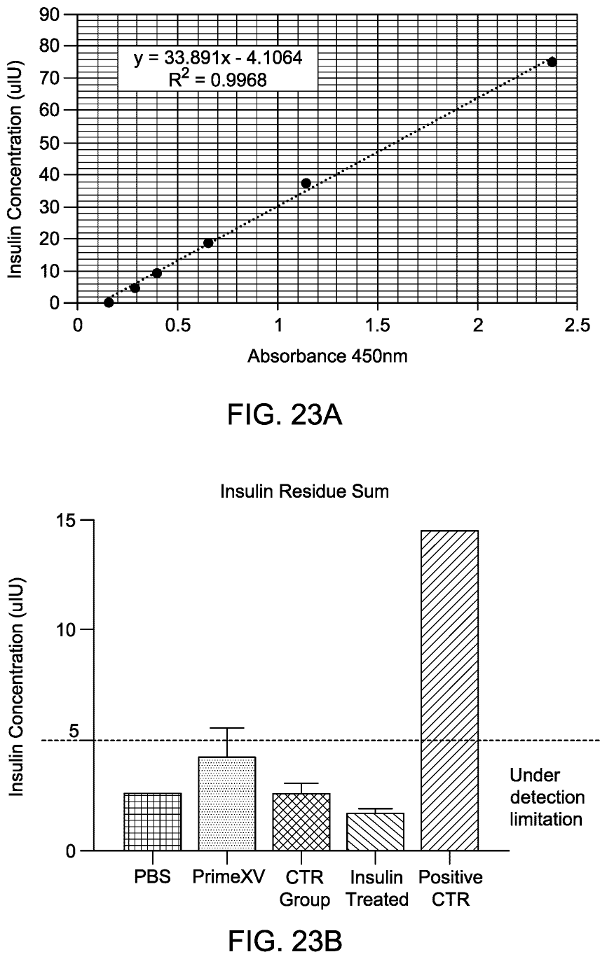

In order to scale up the manufacturing process for clinical scale manufacturing, the process of Example 1 was performed in a clinically relevant scale which is the ability to process a full leukopak and enable clinical trial and commercial therapy. Clinical scale manufacturing used the same media, additives, gene editing reagents and culture timing as described in Example 1. Differences included larger format consumables for G-Rex vessels where volumes of media and additives were scaled linearly and the equipment used for electroporation. Both Lonza Nucleofector and Thermo Xenon electroporation instruments were tested and performed similarly. Electroporation pulse codes were the same for Lonza Nucleofector when using the LV cartridge. Pulse code MK25 was used for the Thermo Xenon instruments. Cells cultured and transfected under GMP processes at a clinically relevant scale showed similar results as obtained in Example 1, as shown in FIGS. 14A to 23B.

TABLE 4
TRAC loci

| | Targeted locus |
|---|---|
| TRAC_1 | chr14: 22547530-22547549 |
| TRAC_2 | chr14: 22547537-22547556 |
| TRAC_3 | chr14: 22547641-22547660 |
| TRAC_4 | chr14: 22547672-22547691 |
| TRAC_5 | chr14: 22550609-22550628 |
| TRAC_6 | chr14: 22550626-22550645 |
| TRAC_7 | chr14: 22547519-22547538 |
| TRAC_8 | chr14: 22547525-22547544 |
| TRAC_9 | chr14: 22547557-22547576 |
| TRAC_10 | chr14: 22549639-22549658 |
| TRAC_11 | chr14: 22549661-22549680 |
| TRAC_12 | chr14: 22550582-22550601 |
| TRAC_13 | chr14: 22550597-22550616 |
| TRAC_14 | chr14: 22550602-22550621 |
| TRAC_15 | chr14: 22550623-22550642 |
| TRAC_16 | chr14: 22550643-22550662 |

TABLE 5
TRBC loci

| | Targeted locus | |
|---|---|---|
| TRBC_1 | chr7: 142801048-142801067 | |
| TRBC_2 | chr7: 142801063-142801082 | |
| TRBC_3 | chr7: 142791758-142791777 | chr7: 142801105-142801124 |
| TRBC_4 | chr7: 142801122-142801141 | |
| TRBC_5 | chr7: 142801140-142801159 | |
| TRBC_6 | chr7: 142801141-142801160 | |
| TRBC_7 | chr7: 142791812-142791831 | chr7: 142801159-142801178 |
| TRBC_8 | chr7: 142791809-142791828 | chr7: 142801156-142801175 |
| TRBC_9 | chr7: 142791821-142791840 | chr7: 142801168-142801187 |
| TRCB_10 | chr7: 142791824-142791843 | chr7: 142801171-142801190 |
| TRBC_11 | chr7: 142791834-142791853 | chr7: 142801181-142801200 |
| TRBC_12 | chr7: 142791836-142791855 | chr7: 142801183-142801202 |
| TRBC_13 | chr7: 142791894-142791913 | chr7: 142801241-142801260 |
| TRBC_14 | chr7: 142791895-142791914 | chr7: 142801242-142801261 |
| TRBC_15 | chr7: 142791899-142791918 | chr7: 142801246-142801265 |
| TRBC_16 | chr7: 142791929-142791948 | chr7: 142801276-142801295 |
| TRBC_17 | chr7: 142791962-142791981 | chr7: 142801309-142801328 |
| TRBC_18 | chr7: 142791977-142791996 | chr7: 142801324-142801343 |
| TRBC_19 | chr7: 142791997-142792016 | chr7: 142801344-142801363 |
| TRBC_20 | chr7: 142792004-142792023 | chr7: 142801351-142801370 |
| TRBC_21 | chr7: 142792043-142792062 | chr7: 142801390-142801409 |
| TRBC_22 | chr7: 142791721-142791740 | chr7: 142801068-142801087 |
| TRBC_23 | chr7: 142791749-142791768 | chr7: 142801096-142801115 |
| TRBC_24 | chr7: 142791808-142791827 | chr7: 142801155-142801174 |
| TRBC_25 | chr7: 142791963-142791982 | chr7: 142801310-142801329 |
| TRBC_26 | chr7: 142792050-142792069 | chr7: 142801397-142801416 |

What is claimed is:

1. A method of editing an endogenous gene in a population of T cells, the method comprising: contacting the population of T cells with a gene editing reagent or a polynucleotide encoding a gene editing reagent under conditions to allow the polynucleotide or gene editing reagent to enter the cell, and culturing the population of T cells in the presence of one or more of, insulin, an insulin analog, an insulin agonist and/or an insulin partial agonist before and/or during, and/or after the contacting step to obtain an engineered population of T cells.

2. The method of claim 1, further comprising contacting the population of T cells with a donor DNA.

3. The method of claim 1 or 2, wherein the polynucleotide encoding the gene editing reagent comprises: single-stranded DNA, double-stranded DNA, a linear DNA strand, a plasmid, a nanoplasmid, or a minicircle.

4. The method of claim 3, wherein the polynucleotide encoding the gene editing reagent comprises a plasmid comprising a plasmid backbone and a polynucleotide sequence encoding the gene editing reagent.

5. The method of claim 4, wherein the plasmid further comprises the donor DNA.

6. The method of any one of claims 2 to 5, wherein the donor DNA sequence comprises a polynucleotide encoding a gene product.

7. The method of claim 6, wherein the population of T cells was obtained from a subject.

8. The method of claim 7, wherein the gene product sequence comprises a chimeric antigen receptor (CAR), a T cell receptor (TCR), a human leukocyte antigen (HLA), or an alloimmune defense receptor (ADR), or a subunit thereof.

9. The method of claim 8, wherein the TCR sequence comprises an exogenous TCR-beta subunit or fragment thereof and/or an exogenous TCR-alpha subunit or fragment thereof, or a chimeric antigen receptor and/or subunit thereof.

10. The method of claim 9, wherein the TCR sequence comprises an exogenous TCR-beta subunit or fragment thereof and an exogenous TCR-alpha subunit or fragment thereof.

11. The method of claim 10, wherein the TCR sequence is inserted in a TRAC or TRBC locus.

12. The method of claim 10, wherein the TCR sequence is inserted in a TRAC locus.

13. The method of claim 10, wherein the TCR sequence is inserted in a TRBC locus.

14. The method of claim 1 or 2, wherein contacting the population of T cells with the gene editing reagent or polynucleotide encoding the gene editing reagent comprises transfecting the population of T cells with the gene editing reagent or polynucleotide encoding the gene editing reagent.

15. The method of claim 14, wherein the transfecting comprises electroporation.

16. The method of claim 14, wherein the transfecting comprises nucleofection.

17. The method of claim 14, wherein the transfecting comprises lipid transfection.

18. The method of claim 14, wherein the transfecting comprises microfluidic transfection.

19. The method of any one of claims 1-18, wherein the population of T cells is cultured in the presence of insulin, an insulin analog, an insulin agonist and/or an insulin partial agonist prior to the contacting step.

20. The method of claim 19, comprising culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step.

21. The method of any one of claims 1-18, wherein the population of T cells is cultured in the presence of insulin, an insulin analog, an insulin agonist and/or an insulin partial agonist after the contacting step.

22. The method of claim 21, comprising culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes after the contacting step.

23. The method of any one of claims 1-18, wherein the population of T cells is cultured in the presence of insulin, an insulin analog, an insulin agonist and/or an insulin partial agonist prior to and after the contacting step.

24. The method of claim 23, comprising culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes prior to the contacting step.

25. The method of claim 23 or 24, comprising culturing the population of T cells in the presence of insulin, an insulin analog, an insulin agonist and/or an insulin partial agonist for 48 hours, 24 hours, 12 hours, 6 hours, 4 hours, 2 hours, 1 hour, or 30 minutes after the contacting step.

26. The method of any one of claims 1-25, wherein the insulin, an insulin analog, an insulin agonist and/or an insulin partial agonist is administered at a concentration of about 1 μg/ml to about 50 μg/ml.

27. The method of claim 26, wherein the insulin, an insulin analog, an insulin agonist and/or an insulin partial agonist is administered at a concentration of about 1 μg/ml, about 5 μg/ml, or about 25 μg/ml.

28. The method of claim 27, wherein the population of T cells is transfected with the donor DNA and the gene editing agent or the polynucleotide encoding the gene editing reagent simultaneously.

29. The method of any one of claims 1-28, wherein the gene editing reagent comprises an RNA-guided nuclease.

30. The method of claim 29, wherein the RNA-guided nuclease is a CRISPR-Cas system.

31. The method of claim 30, wherein the CRISPR-Cas system comprises a Cas9 or a Cas9 variant.

32. The method of any one of claims 1-31, wherein the gene editing reagent comprises a CRISPR-Cas system comprising a Cas protein and a guide RNA.

33. The method of any one of claims 1-32, wherein at least 80% of engineered T cells are T central memory (TCM) and/or T stem cell memory (TSCM).

34. A method of monitoring of cell viability for an engineered population of T cells, the method comprising measuring mitochondrial function and cell metabolism over time.

35. The method of claim 34, wherein the mitochondrial membrane potential is measured.

36. The method of claim 35, wherein the mitochondrial membrane potential is measured using a dye-based assay.

37. The method of claim 36, wherein the dye is JC-1 or JC-10.

38. A method of monitoring cell viability for an engineered population of T cells, the method comprising measuring a cell metabolism marker over time.

39. The method of claim 38, wherein the method comprises measuring changes in glucose metabolism, Bcl-2 expression, Bcl-XL expression, Bax expression, or Bad expression over time.

40. The method of claim 39, wherein the engineered population of T cells glucose metabolism is monitored using a glucose analog.

41. The method of claim 40, wherein the analog is 2-NBDG.

42. A method of increasing cell viability of a population of engineered T cells, comprising contacting a population of T cells in the presence of insulin, insulin analog, insulin agonist, an insulin partial agonist, a gene editing reagent, or a polynucleotide encoding a gene editing reagent thereby forming the population of engineered T cells, wherein the population of engineered T cells has increased cell viability, growth, and/or gene editing efficiencies relative to a population of engineered T cells that are not contacted with insulin, an insulin analog, an insulin agonist, or an insulin partial agonist, wherein the population of engineered T cells are administered to a subject in need thereof.

43. The method of claim 42, further comprising contacting the population of T cells with a donor DNA.

44. The method of claim 42 or 43, wherein the polynucleotide comprises: single-stranded DNA, double-stranded DNA, a linear DNA strand, a plasmid, a nanoplasmid, or minicircle.

45. The method of claim 44, wherein the polynucleotide comprises a plasmid comprising a plasmid backbone and a polynucleotide sequence encoding the gene editing reagent.

46. The method of claim 45, wherein the plasmid further comprises the donor DNA.

47. The method of any one of claims 43 to 46, wherein the donor DNA sequence comprises a polynucleotide encoding a gene product.

48. The method of claim 47, wherein the gene product is autologous or allogeneic to the subject.

49. The method of claim 47, wherein the gene product sequence comprises a chimeric antigen receptor (CAR), a T cell receptor (TCR), a human leukocyte antigen (HLA), or an alloimmune defense receptor (ADR), or a subunit thereof.

50. The method of claim 49, wherein the TCR sequence comprises an exogenous TCR-beta subunit or fragment thereof and/or an exogenous TCR-alpha subunit or fragment thereof, or a chimeric antigen receptor and/or subunit thereof.

51. The method of claim 50, wherein the TCR sequence comprises an exogenous TCR-beta subunit or fragment thereof and an exogenous TCR-alpha subunit or fragment thereof.

52. The method of claim 51, wherein the TCR sequence is inserted in a TRAC or TRBC locus.

53. The method of claim 51, wherein the TCR sequence is inserted in a TRAC locus.

54. The method of claim 51, wherein the TCR sequence is inserted in a TRBC locus.

55. The method of claim 42 or 43, wherein contacting the population of T cells with the gene editing reagent or polynucleotide encoding the gene editing reagent comprises transfecting the population of T cells with the gene editing reagent or polynucleotide encoding the gene editing reagent.

56. The method of claim 55, wherein the transfecting comprises electroporation.

57. The method of claim 55, wherein the transfecting comprises nucleofection.

58. The method of claim 55, wherein the transfecting comprises lipid transfection.

59. The method of claim 55, wherein the transfecting comprises microfluidic transfection.

60. The method of any one of claims 42-59, wherein the population of T cells is cultured in the presence of insulin, an insulin analog, an insulin agonist, and/or an insulin partial agonist, prior to the contacting step.

61. The method of claim 60, comprising culturing the population of T cells in the presence of insulin, an insulin analog, an insulin agonist, and/or an insulin partial agonist, for 48 hours, 24 hours, 12 hours, 6 hours, 4 hours, 2 hours, 1 hour, or 30 minutes prior to the contacting step.

62. The method of any one of claims 42-59, wherein the population of T cells is cultured in the presence of insulin, an insulin analog, an insulin agonist, and/or an insulin partial agonist, after the contacting step.

63. The method of claim 62, comprising culturing the population of T cells in the presence of insulin, an insulin analog, an insulin agonist, and/or an insulin partial agonist, for 48 hours, 24 hours, 12 hours, 6 hours, 4 hours, 2 hours, 1 hour, or 30 minutes after the contacting step.

64. The method of any one of claims 42-59, wherein the population of T cells is cultured in the presence of insulin, an insulin analog, an insulin agonist, and/or an insulin partial agonist, prior to and after the contacting step.

65. The method of claim 64, comprising culturing the population of T cells in the presence of insulin, an insulin analog, an insulin agonist, and/or an insulin partial agonist, for 48 hours, 24 hours, 12 hours, 6 hours, 4 hours, 2 hours, 1 hour, or 30 minutes prior to the contacting step.

66. The method of claim 64 or 65, comprising culturing the population of T cells in the presence of insulin, an insulin analog, an insulin agonist, and/or an insulin partial agonist, for 48 hours, 24 hours, 12 hours, 6 hours, 4 hours, 2 hours, 1 hour, or 30 minutes after the contacting step.

67. The method of any one of claims 42 to 66, wherein the insulin, an insulin analog, an insulin agonist, and/or an insulin partial agonist, is administered at a concentration of about 1 μg/ml to about 50 μg/ml.

68. The method of claim 67, wherein the insulin, an insulin analog, an insulin agonist, and/or an insulin partial agonist, is administered at a concentration of about 1 μg/ml, about 5 μg/ml, or about 25 μg/ml.

69. The method of claim 68, wherein the population of T cells is transfected with the donor DNA and the gene editing agent or the polynucleotide encoding the gene editing reagent simultaneously.

70. The method of any one of claims 42-69, wherein the gene editing reagent comprises an RNA-guided nuclease.

71. The method of claim 70, wherein the RNA-guided nuclease is a CRISPR-Cas system.

72. The method of claim 71, wherein the CRISPR-Cas system comprises a Cas9 or a Cas9 variant.

73. The method of any one of claims 42-72, wherein the gene editing reagent comprises a CRISPR-Cas system comprising a Cas protein and a guide RNA.

74. The method of any one of claims 42-73, wherein at least 80% of engineered T cells are TCM and/or TSCM.

75. The method of any one of claims 42-74, wherein the cell viability and culture performance for the engineered population of T cells is monitored, the method comprising measuring mitochondrial function and cell metabolism over time.

76. The method of claim 75, wherein the mitochondrial membrane potential is measured.

77. The method of claim 76, wherein the mitochondrial membrane potential is measured using a dye-based assay.

78. The method of claim 77, wherein the dye is JC-1 or JC-10.

79. The method of any one of claims 42-74, wherein the cell viability and culture performance for a population of T cells is monitored, the method comprising measuring a cell metabolism marker overtime.

80. The method of claim 79, wherein the method comprises measuring changes in glucose metabolism over time.

81. The method of claim 80, wherein the engineered population of T cells glucose metabolism is monitored using a glucose analog.

82. The method of claim 81, wherein the analog is 2-NBDG.

83. The method of any one of claims 75-82, wherein the cell viability of the population of engineered T cells is increased from at least about 0.1 fold to at least about 5.0 fold relative to a population of engineered T cells that are not cultured in the presence of insulin, an insulin analog, an insulin agonist, or an insulin partial agonist.

84. The method of claim 83, wherein the cell viability is increased about 2.0 fold.

85. The method of any one of claims 75-83, wherein the cell viability of the population of engineered T cells is from about 30% to about 95%.

86. A method of increasing gene editing efficiency in a population of engineered T cells, comprising contacting a population of T cells with insulin, an insulin analog, an insulin agonist, an insulin partial agonist, a gene editing reagent, and a polynucleotide, thereby forming the population of engineered T cells, wherein the population of engineered T cells has increased gene editing efficiency relative to a population of engineered T cells that are not contacted with insulin, insulin analog, insulin agonist, or insulin partial agonist.

87. The method of claim 86, wherein the contacting the population of T cells with the polynucleotide comprises transfecting the population of T cells with the polynucleotide.

88. The method of claim 86 or 87, wherein the polynucleotide is a donor DNA.

89. The method of any one of claims 86-88, wherein the polynucleotide comprises: single-stranded DNA, double-stranded DNA, a linear DNA strand, a plasmid, a nanoplasmid, or a minicircle.

90. The method of any one of claims 86-89, further comprising contacting the population of T cells with a gene editing reagent.

91. The method of claim 90, wherein contacting the population of T cells with the gene editing reagent comprises transfecting the population of T cells with the gene editing reagent or a polynucleotide encoding the gene editing reagent.

92. The method of claim 91, wherein the population of T cells is transfected with the polynucleotide and the gene editing agent or the polynucleotide encoding the gene editing reagent simultaneously.

93. The method of any one of claims 86-92, wherein the population of T cells is contacted with about 1 μg/ml to about 50 μg/ml of insulin, an insulin analog, an insulin agonist, and/or an insulin partial agonist.

94. The method of any one of claims 86-93, wherein the population of T cells is contacted simultaneously with the polynucleotide and insulin, an insulin analog, an insulin agonist, and/or an insulin partial agonist.

95. The method of any one of claims 86-94, wherein the population of T cells is contacted sequentially with the polynucleotide and insulin, an insulin analog, an insulin agonist, and/or an insulin partial agonist.

96. The method of claim 95, wherein the population of T cells is contacted with insulin inhibitors prior to the polynucleotide.

97. The method of any one of claims 86-96, wherein the gene editing efficiency of the population of engineered T cells is increased from at least about 0.1 fold to at least about 5 fold relative to a population of engineered T cells that are not contacted with insulin, an insulin analog, an insulin agonist, and/or an insulin partial agonist.

98. The method of claim 97, wherein the gene editing efficiency of the population of engineered T cells is increased from about 2.0 fold to about 3.0 fold.

99. The method of any one of claims 86-98, wherein the gene editing efficiency of the population of engineered T cells is from about 1% to about 99%.

100. The method of any one of claims 86-99, wherein knock-out efficiency of the population of engineered T cells is from about 70% to about 99%.

101. The method of claim 100, wherein the knock-out efficiency is about 90%.

102. The method of any one of claims 86-101, wherein knock-in efficiency of the population of engineered T cells is from about 20% to about 99%.

103. The method of claim 102, wherein the knock-in efficiency is about 60%.

104. A method for increasing expansion of a population of engineered T cells, comprising
(i) contacting a population of T cells with insulin, an insulin analog, an insulin agonist, and/or an insulin partial agonist, and a polynucleotide, thereby forming the population of engineered T cells, and
(ii) expanding the population of engineered T cells, thereby forming a population of expanded engineered T cells,
wherein the insulin, insulin analog, insulin agonist, and/or insulin partial agonist increases the population of expanded engineered T cells relative to a population of engineered T cells that are not contacted with insulin, insulin analog, insulin agonist, and/or insulin partial agonist.

105. The method of claim 104, wherein the contacting the population of T cells with the polynucleotide comprises transfecting the population of T cells with the polynucleotide.

106. The method of claim 104 or 105, wherein the polynucleotide is a donor DNA.

107. The method of any one of claims 104-106, wherein the polynucleotide comprises: single-stranded DNA, double-stranded DNA, a linear DNA strand, a plasmid, a nanoplasmid, or a minicircle.

108. The method of any one of claims 104-107, further comprising contacting the population of T cells with a gene editing reagent.

109. The method of claim 108, wherein contacting the population of T cells with the gene editing reagent comprises transfecting the population of T cells with the gene editing reagent or a polynucleotide encoding the gene editing reagent.

110. The method of claim 109, wherein the population of T cells is transfected with the polynucleotide and the gene editing agent or the polynucleotide encoding the gene editing reagent simultaneously.

111. The method of any one of claims 104-110, wherein the population of T cells is contacted with about 1 μg/ml to about 50 μg/ml of insulin, insulin analog, insulin agonist, and/or insulin partial agonist.

112. The method of any one of claims 104-111, wherein the population of T cells is contacted simultaneously with the polynucleotide and insulin, insulin analog, insulin agonist, and/or insulin partial agonist.

113. The method of any one of claims 104-111, wherein the population of T cells is contacted sequentially with the polynucleotide and insulin, insulin analog, insulin agonist, and/or insulin partial agonist.

114. The method of claim 113, wherein the population of T cells is contacted with insulin, insulin analog, insulin agonist, and/or insulin partial agonist prior to contacting with the polynucleotide.

115. The method of claim 113, wherein the population of T cells is contacted with insulin, insulin analog, insulin agonist, and/or insulin partial agonist after contacting with the polynucleotide.

116. The method of claim 113, wherein the population of T cells is contacted with insulin, insulin analog, insulin agonist, and/or insulin partial agonist prior to and after contacting with the polynucleotide.

117. The method of any one of claims 104-116, wherein the population of expanded engineered T cells is increased from at least about 0.1 fold to at least about 5.0 fold relative to a population of engineered T cells that are not contacted with insulin, insulin analog, insulin agonist, and/or insulin partial agonist.

118. The method of claim 117, wherein the population of expanded engineered T cells is increased from about 2.0 fold to about 3.0 fold.

119. The method of any one of claims 104-118, wherein the population of engineered T cells are expanded from at least about 0.1 fold to at least about 1000 fold.

120. The method of claim 119, wherein the engineered T cells are expanded about 20 fold.

121. An engineered population of T cells, made by the method of any one of claims 1-120.

122. A pharmaceutical composition comprising the engineered T cell of claim 121.

123. A method of treating a disease in a subject in need thereof, comprising administering a therapeutically effective amount of the engineered T cell of claim 96 or the pharmaceutical composition of claim 97.

124. The method of claim 123, wherein the disease is cancer.

125. The method of claim 123 or 124, wherein the cancer is leukemia, lymphoma, carcinoma, sarcoma, brain cancer, glioma, glioblastoma, neuroblastoma, prostate cancer, colorectral cancer, pancreatic cancer, medulloblastoma, melanoma, cervical cancer, gastric cancer, ovarian cancer, lung cancer, cancer of the head and neck, breast cancer, liver cancer, or uterine cancer.
